(19) United States
(12) Patent Application Publication
(10) Pub. No.: US 2011/0076681 A1
(43) Pub. Date: Mar. 31, 2011

(54) Modified gene-silencing nucleic acid molecules and uses thereof

(75) Inventors: Peter Michael Waterhouse, O'Connor (AU); Linda Jane Lockett, Denistone (AU); Ming-Bo Wang, Kaleen (AU); Timothy James Doran, Ocean Grove (AU); Robert John Moore, Ascot Vale (AU); Gerald Wayne Both, North Ryde (AU)

(73) Assignee: Commonwealth Scientific and Industrial Research Organisation

(21) Appl. No.: 12/798,247

(22) Filed: Mar. 31, 2010

Related U.S. Application Data
(63) Continuation of application No. 10/571,384, filed on Jun. 1, 2006, now abandoned.
(60) Provisional application No. 60/502,250, filed on Sep. 12, 2003.

(30) Foreign Application Priority Data
Sep. 12, 2003 (AU) 2003904990

(51) Int. Cl. C12N 5/07; C12N 1/14; C12N 1/00; C12Q 1/68; C12N 5/071; C07H 21/00

(52) U.S. Cl. 435/6; 435/375; 435/254.1; 435/243; 435/366; 435/352; 435/354; 435/353; 435/363; 435/348; 536/24.5; 435/325

(57) ABSTRACT

Methods and means for efficiently downregulating the expression of a target gene of interest in cell from an organism that is an animal, fungus, and protest. The invention provides chimeric nucleic acid molecules for downregulating target genes. The invention also provides modified cells and organisms comprising the chimeric nucleic acid molecules and compositions comprising the chimeric molecules.

This application is a continuation of U.S. Ser. No. 10/571,384, now abandoned, which is a §371 National Stage of PCT International Application No. PCT/AU2004/001237, filed Sep. 10, 2004, claiming priority of U.S. Provisional Application No. 60/502,250, filed Sep. 12, 2003, and Australian Patent Application No. 2003904990, filed Sep. 12, 2003 the contents of all of which are hereby incorporated by reference.

### TECHNICAL FIELD

The present invention relates to methods for efficiently downregulating the expression of any gene of interest in an animal, fungal or protist cell. To this end, the invention provides modified antisense and sense RNA or nucleic acid molecules, chimeric nucleic acid molecules encoding such modified antisense or sense RNA or nucleic acid molecules. The invention also provides cells or organisms such as, animals, fungi or protists comprising the modified antisense and/or sense RNA or nucleic acid molecules or the encoding chimeric nucleic acid molecules.

### BACKGROUND OF THE INVENTION

Recently, it has been shown that introduction of double stranded RNA (dsRNA), also called interfering RNA (RNAi) or hairpin RNA, is an effective trigger for the induction of gene-silencing in a large number of eukaryotic organisms. The mechanism by which this process is thought to occur is shown schematically in FIG. 1, resulting in the sequence-specific degradation and therefore inactivation or “silencing” of a target gene RNA which may be a viral RNA. Such RNA-mediated gene silencing is thought to have a multitude of potential applications in animals, fungi and plants, including the use as a tool for functional genomics, in identifying gene function, and in the treatment of diseases in plants and animals, including humans.

Both the qualitative level of dsRNA mediated gene silencing (level of gene-silencing within an organism) and the quantitative level (number of organisms showing a significant level of gene-silencing within a population) have proven superior to the more conventional antisense RNA or sense RNA mediated gene silencing methods. One way the dsRNA can be delivered to a cell is by means of a transgene comprising an inverted repeat sequence, where expression of the transgene in the nucleus of the cell results in production of a dsRNA (ie hairpin RNA) which has a high degree of sequence identity in at least a portion of the dsRNA, over at least 19 nucleotides in one strand, with a region of the target RNA. The dsRNA may be processed into 21-23 nucleotide dsRNA portions (also called siRNAs) which are transferred to the cytoplasm where they may be effective in post-transcriptional gene silencing (PTGS) of the target gene RNA (FIG. 2).

For practical purposes, the production of antisense RNA molecules and chimeric genes encoding such antisense RNA is more straightforward than the production of dsRNA molecules or the encoding genes. Indeed, the chimeric nucleic dsRNA molecules or the encoding genes contain large, more or less perfect inverted repeat structures, and such structures tend to hamper the intact maintenance of these nucleic acids in the intermediate prokaryotic cloning hosts. Furthermore, the production in mammalian cells of dsRNAs, at least those having a length greater than 30 basepairs in the double stranded portion, may result in the induction of non-sequence-specific responses such as the induction of interferon responses or apoptosis. The methods and means as hereinafter described to increase the efficiency of antisense-RNA mediated gene silencing provide a solution to these problems as described in the different embodiments and claims.

U.S. Pat. No. 5,190,131 and EP 0 467 349 A1 describe methods and means to regulate or inhibit gene expression in a cell by incorporating into or associating with the genetic material of the cell a non-native nucleic acid sequence. The sequence is transcribed to produce a mRNA which is complementary to and capable of binding to the mRNA produced by the genetic material of that cell.

EP 0 223 399 A1 describes methods to effect useful somatic changes in plants by causing the transcription in the plant cells of negative RNA strands which are substantially complementary to a target RNA strand. The target RNA strand can be a mRNA transcript created in gene expression, a viral RNA, or other RNA present in the plant cells. The negative RNA strand is complementary to at least a portion of the target RNA strand to inhibit its activity in vivo.

EP 0 240 208 describes a method to regulate expression of genes encoded for in plant cell genomes, achieved by integration of a gene under the transcriptional control of a promoter which is functional in the host. In this method, the transcribed strand of DNA is complementary to the strand of DNA that is transcribed from the endogenous gene(s) one wishes to regulate.

WO95/15394 and U.S. Pat. No. 5,908,779 describe a method and construct for regulating gene expression through inhibition by nuclear antisense RNA in (mouse) cells. The construct comprises a promoter, antisense sequences, and a cis- or trans-ribozyme which generates 3′-ends independently of the polyadenylation machinery and thereby inhibits the transport of the RNA molecule to the cytoplasm.

WO98/05770 discloses antisense RNA with special secondary structures such as (GC)ₙ-palindrome-(GC)ₙ or (AT)ₙ-palindrome-(AT)ₙ or (CG)ₙ-palindrome-(CG)ₙ and the like.

WO 01/12824 discloses methods and means for reducing the phenotypic expression of a nucleic acid of interest in eukaryotic cells, particularly in plant cells, by providing aberrant, preferably unpolyadenylated, target-specific RNA to the nucleus of the host cell. In an embodiment, the unpolyadenylated target-specific RNA is provided by transcription of a chimeric gene comprising a promoter, a DNA region encoding the target-specific RNA, a self-splicing ribozyme and a DNA region involved in 3′ end formation and polyadenylation.

WO 02/10365 provides a method for gene suppression in eukaryotes by transformation with a recombinant construct containing a promoter, at least one antisense and/or sense nucleotide sequence for the gene(s) to be suppressed, wherein the nucleus-to-cytoplasm transport of the transcription products of the construct is inhibited. In one embodiment, nucleus-to-cytoplasm transport is inhibited by the absence of a normal 3′ UTR. The construct can optionally include at least one self-cleaving ribozyme. The construct can also optionally include sense and/or antisense sequences to multiple genes that are to be simultaneously down regulated using a single promoter. Also disclosed are vectors, plants, animals, seeds, gametes, and embryos containing the recombinant constructs.

Zhao et al., J. Gen. Virology, 82, 1491-1497 (2001) described the use of a vector based on Potato Virus X in a whole plant assay to demonstrate nuclear targeting of Potato Spindle Tuber Viroid (PSTVd).

WO 02/00894 relates to gene silencing methods wherein the nucleic acid constructs comprise within the transcribed region a DNA sequence which consists of a stretch of T bases in the transcribed strand.

WO 02/00904 relates to gene silencing methods wherein nucleic acid constructs comprise (or encode) homology to at least one target mRNA expressed by a host, and in the proximity thereto, two complementary RNA regions which are unrelated to any endogenous RNA in the host.

PCT/AU03/00292 teaches a general method of modifying gene silencing RNA by attachment to nuclear localization signals, but does not teach the application of this method for down regulating target genes in a cell of an animal, fungus or protist. In particular, the document does not teach the use of target genes involved in animal disease or animal function.

Accordingly, there remains a need for providing effective methods and compositions for down regulating the expression of target genes in a cell of an animal, fungus or protist.

### SUMMARY OF THE INVENTION

In a first aspect of the present invention there is provided a method of down regulating the expression of a target gene in a cell of an animal, fungus or protist, the method comprising the step of providing the cell with a chimeric nucleic acid molecule, wherein the molecule comprises

a) a target-gene specific region comprising a nucleotide sequence of at least about 16 consecutive nucleotides having at least about 94% sequence identity with the complement of 16 consecutive nucleotides from a transcribed nucleotide sequence of the target gene, and
b) a largely double stranded nucleic acid region,
wherein the target gene is a reporter gene, a pathogenic animal virus gene, a cancer-related gene, an oncogene, an immunomodulatory gene, a gene encoding a cytokine, growth factor, enzyme or a transcription factor or an animal disease causing gene.

In an embodiment, the chimeric nucleic acid molecule is an RNA molecule. It is preferred that the cell is an animal cell. The largely double stranded nucleic acid region of the chimeric nucleic acid molecule preferably comprises a nuclear localization signal. The largely double stranded nucleic acid region may comprise a nucleotide sequence obtained from a viroid of the Potato Spindle Tuber Viroid (PSTVd)-type, a nucleotide sequence comprising at least 35 repeats of a trinucleotide CUG, CAG, GAC or GUC, a nucleotide sequence obtained from hepatitis delta RNA, or a synthetic nucleotide sequence comprising a nucleic acid-nuclear localization signal. The viroid can have a nucleotide sequence of SEQ ID No 3, SEQ ID No 4, SEQ ID No 5, SEQ ID No 6, SEQ ID No 7 or SEQ ID No 8. The largely double stranded nucleic acid region can comprise a viroid genome nucleotide sequence of the genome nucleotide sequence of a viroid.

In an embodiment, the largely double stranded nucleic region comprises a RNA sequence having at least 35 repeats, more preferably between 44 and 2000 repeats of the trinucleotide CUG of the trinucleotide CUG. The chimeric nucleic acid molecule preferably comprises multiple target-gene specific regions. The chimeric nucleic acid molecule preferably comprises an intron sequence. The chimeric nucleic acid is preferably a RNA molecule produced by transcription of a chimeric DNA molecule.

In another preferred embodiment, the largely double stranded nucleic region comprises a nucleotide sequence obtained from a small nuclear RNA (snRNA). In an embodiment, the largely double stranded nucleic acid region comprises a nucleotide sequence obtained from a small nuclear RNA (snRNA) such as U3, U2, U4 to U6, U8, U13 to U16, U18 to U21, U23 to U72, 4.5S RNAI to III, 5S RNAIII, E2 or E3. The largely double stranded nucleic acid region preferably comprises a nucleotide sequence obtained from a small nuleaolar localised RNA (snoRNA). In an embodiment of the invention, the largely double stranded nucleic acid region comprises a nucleotide sequence obtained from U6 snoRNA, most preferably from human U6 snoRNA as shown in FIG. 16.

The method of the invention preferably, further comprises the step of identifying a cell of an animal, fungus or protist, wherein the expression of the target gene is down regulated.

In a second aspect of the invention there is provided a chimeric nucleic acid molecule for down regulating the expression of a target gene in a cell of an animal, fungus or protist, wherein the molecule comprises

a) a target-gene specific region comprising a nucleotide sequence of at least about 16 consecutive nucleotides having at least about 94% sequence identity with the complement of 16 consecutive nucleotides from a transcribed nucleotide sequence of the target gene, and
b) a largely double stranded nucleic acid region,
wherein the target gene is a reporter gene, a pathogenic animal virus gene, a cancer-related gene, an oncogene, an immunomodulatory gene, a gene encoding a cytokine, growth factor, enzyme or a transcription factor or an animal disease causing gene. The chimeric nucleic acid molecule is preferably a RNA molecule. The largely double stranded nucleic acid region of the chimeric nucleic acid molecule preferably comprises a nuclear localization signal. The largely double stranded nucleic acid region can comprise a nucleotide sequence obtained from a viroid of the Potato Spindle Tuber Viroid (PSTVd)-type, a nucleotide sequence comprising at least 35 repeats of a trinucleotide CUG, CAG, GAC or GUC, a nucleotide sequence obtained from hepatitis delta RNA, or a synthetic nucleotide sequence comprising a nucleic acid-nuclear localization signal. The viroid can have a nucleotide sequence of SEQ ID No 3, SEQ ID No 4, SEQ ID No 5, SEQ ID No 6, SEQ ID No 7 or SEQ ID No 8.

The chimeric nucleic acid molecule comprises a largely double stranded nucleic acid region and may comprise a viroid genome nucleotide sequence of the genome nucleotide sequence of a viroid. In an embodiment, the largely double stranded nucleic region comprises a RNA sequence having at least 35 repeats, more preferably between 44 and 2000 repeats of the trinucleotide CUG of the trinucleotide CUG. The chimeric nucleic acid molecule preferably comprises multiple target-gene specific regions. The chimeric nucleic acid molecule preferably comprises an intron sequence. The chimeric nucleic acid is preferably a RNA molecule produced by transcription of a chimeric DNA molecule.

In a third aspect of the invention there is provided a chimeric DNA molecule for down regulating the expression of a target gene in a cell of an animal, fungus or protist, the chimeric DNA comprising

a) a promoter or promoter region recognizable by RNA polymerases in the cell; operably linked to
b) a DNA region which when transcribed yields a RNA molecule, wherein the RNA molecule comprises
(i) a target-gene specific region comprising a nucleotide sequence of at least about 16 consecutive nucleotides having at least about 94% sequence identity with the complement of 16 consecutive nucleotides from a transcribed nucleotide sequence of the target gene, and
(ii) a largely double stranded nucleic acid region,
wherein the target gene is a reporter gene, a pathogenic animal virus gene, a cancer-related gene, an oncogene, an immunomodulatory gene, a gene encoding a cytokine, growth factor, enzyme or a transcription factor or an animal disease causing gene.

The chimeric DNA molecule preferably comprises a transcription termination and/or polyadenylation signal operably linked to the DNA region which when transcribed yields the RNA molecule. In an embodiment, the promoter or promoter region of the chimeric DNA functions in an animal cell. The promoter or promoter region is preferably a promoter recognized by a prokaryotic RNA polymerase such as a bacteriophage RNA polymerase.

Depending on the host organism, the promoter or promoter region may a promoter which functions in animals, or a promoter which functions in yeast including fungi or molds. The promoter may also be a promoter or promoter region recognized by a single subunit bacteriophage RNA polymerase. In an embodiment, the chimeric DNA molecule which when expressed in a cell of an animal, fungus or protist down regulates the expression of the target gene.

A fourth aspect of the invention is a cell of an animal, fungus or protist comprising the chimeric DNA molecule of the present invention or comprising the chimeric nucleic acid molecule as hereinbefore described. In an embodiment, the cell is in vitro. The cell is preferably an animal cell an isolated human cell an in vitro human cell, a non-human vertebrate cell, a non-human mammalian cell, fish cell, cattle cell, goat cell, pig cell, sheep cell, rodent cell, hamster cell, mouse cell, rat cell, guinea pig cell, rabbit cell, non-human primate cell, nematode cell, shellfish cell, prawn cell, crab cell, lobster cell, insect cell, fruit fly cell, Coleapteran insect cell, Dipteran insect cell, Lepidopteran insect cell or Homeopteran insect cell.

In another embodiment of the invention there is provided a transgenic, non-human animal, fungus or protist comprising cells having a chimeric nucleic acid molecule or a chimeric DNA molecule as hereinbefore described. The present invention also provides the use of a chimeric nucleic acid molecule or a chimeric DNA molecule as hereinbefore described for down regulating the expression of a target gene in a cell of an animal, fungus or protist.

A further aspect of the invention is a method of producing a transgenic, non-human animal wherein expression of a target gene in cells of the animal is down regulated, the method comprising the steps of:

(a) providing a chimeric nucleic acid molecule or a chimeric DNA molecule as hereinbefore described to at least one cell of the animal;
(b) growing or regenerating a transgenic, non-human animal from said at least one cell of the animal.

The invention also provides a method of producing a transgenic fungal or protest organism wherein expression of a target gene in cells of the organism is down regulated, the method comprising the steps of:

(a) providing a chimeric nucleic acid molecule or a chimeric DNA molecule as hereinbefore described to at least one cell of the organism;
(b) growing or regenerating a transgenic organism from said at least one cell of the organism.

In an another aspect of the invention there is provided a method for down regulating the expression of a target gene in a cell of an animal, fungus or protest comprising, the method comprising the step of providing the cell with a first and a second chimeric nucleic acid molecule,

wherein the first chimeric nucleic acid molecule comprises an antisense target-gene specific nucleic acid region comprising a nucleotide sequence of at least about 19 consecutive nucleotides having at least about 94% sequence identity with the complement of 19 consecutive nucleotides from transcribed nucleotide sequence of the target gene; and the second chimeric nucleic acid molecule comprises a sense target-gene specific nucleic acid region comprising a nucleotide sequence of at least about 19 consecutive nucleotides having at least about 94% sequence identity to the complement of the first chimeric nucleic acid molecule; and the first and second chimeric nucleic acid molecules are capable of basepairing at least between the 19 consecutive nucleotides of the first chimeric nucleic acid molecule and the 19 consecutive nucleotides of the second chimeric nucleic acid molecule; and either the first or the second chimeric nucleic acid molecule comprises a largely double stranded nucleic acid region operably linked to the antisense target-specific nucleic acid region or to the sense target-specific nucleic acid region.

Preferably, the first and the second chimeric nucleic acid molecules both comprise a largely double stranded nucleic acid region. In an embodiment of the invention, the first and the second chimeric nucleic acid molecules comprise the same largely double stranded nucleic acid region. The first and second chimeric nucleic acid molecules both preferably comprise multiple antisense or sense target-gene specific regions. In an embodiment of the invention, the first and second chimeric nucleic acid molecules are RNA molecules which are transcribed from a first and second chimeric gene.

A further aspect of the invention is a cell of an animal, fungus or protist comprising a first and a second chimeric nucleic acid molecule, wherein the first chimeric nucleic acid molecule comprises an antisense target-gene specific nucleic acid region comprising a nucleotide sequence of at least about 19 consecutive nucleotides having at least about 94% sequence identity with the complement of 19 consecutive nucleotides from transcribed nucleotide sequence of the target gene; and

the second chimeric nucleic acid molecule comprises a sense target-gene specific nucleic acid region comprising a nucleotide sequence of at least about 19 consecutive nucleotides having at least about 94% sequence identity to the complement of the first chimeric nucleic acid molecule; and
the first and second chimeric nucleic acid molecules are capable of basepairing at least between the 19 consecutive nucleotides of the first chimeric nucleic acid molecule and the 19 consecutive nucleotides of the second chimeric nucleic acid molecule; and
either the first or the second chimeric nucleic acid molecule comprises a largely double stranded nucleic acid region operably linked to the antisense target-specific nucleic acid region or to the sense target-specific nucleic acid region.

In an embodiment, the first and the second chimeric nucleic acid molecules both comprise a largely double stranded nucleic acid region. The first and the second chimeric nucleic acid molecules preferably comprise the same largely double stranded nucleic acid region. The first and second chimeric nucleic acid molecules preferably comprise multiple antisense or sense target-gene specific regions. The first and second chimeric nucleic acid molecules are most preferably RNA molecules which are transcribed from a first and second chimeric gene.

The present invention also provides a non-human cell of an animal, fungus or protist comprising the modified cells as hereinbefore described.

In a further aspect of the invention there is provided a chimeric sense nucleic acid molecule for down regulating expression of a target gene in a cell of an animal, fungus or protist in cooperation with a chimeric antisense nucleic acid molecule, the chimeric sense nucleic acid molecule comprising

(a) a sense target-gene specific nucleic acid region comprising a nucleotide sequence of at least about 19 consecutive nucleotides having at least about 94% sequence identity to a transcribble nucleotide sequence of the target gene; and
(b) a largely double stranded nucleic acid region.

The chimeric sense nucleic acid molecule preferably comprises a largely double stranded nucleic acid region comprising a nucleotide sequence obtained from a viroid of the Potato Spindle Tuber Viroid (PSTVd)-type, a nucleotide sequence comprising at least 35 repeats of a trinucleotide wherein the trinucleotide is CUG, CAG, GAC or GUC, a nucleotide sequence obtained from hepatitis delta RNA, or a synthetic nucleotide sequence comprising a nucleic acid-nuclear localization signal. In an embodiment, the viroid has a genome nucleotide sequence of SEQ ID No 3, SEQ ID No 4, SEQ ID No 5, SEQ ID No 6, SEQ ID No 7 or SEQ ID No 8. In an embodiment of the invention, the nucleotide sequence comprises a nucleic acid-nuclear localization signal from Potato Spindle Tuber Viroid. The chimeric sense nucleic acid molecule may comprise a viroid genome nucleotide sequence.

The chimeric sense nucleic acid molecule comprises a largely double stranded nucleic region preferably comprising a RNA sequence having at least 35 repeats of the trinucleotide CUG. In an embodiment, the largely double stranded nucleic acid region comprises between 44 and 2000 repeats of the trinucleotide CUG. The chimeric sense nucleic acid molecule preferably comprises multiple target-gene specific regions. The chimeric sense nucleic acid molecule can preferably comprises both an antisense and a sense target-gene specific region. In an embodiment, the chimeric sense nucleic acid molecule comprises an intron sequence.

In a preferred embodiment, the methods and molecules of the present invention, preferably comprise a largely double stranded nucleic region comprises a nucleotide sequence obtained from a small nuclear RNA (snRNA). In an embodiment, the largely double stranded nucleic acid region comprises a nucleotide sequence obtained from a small nuclear RNA (snRNA) that is U3, U2, U4 to U6, U8, U13 to U16, U18 to U21, U23 to U72, 4.5S RNAI to III, 5S RNAIII, E2 or E3. The largely double stranded nucleic acid region preferably comprises a nucleotide sequence obtained from a small nuleaolar localised RNA (snoRNA). In an embodiment of the invention, the largely double stranded nucleic acid region comprises a nucleotide sequence obtained from U6 snoRNA, most preferably from human U6 snoRNA as shown in FIG. 16.

In yet another aspect of the invention there is provided a chimeric DNA molecule for down regulating the expression of a target gene in a a cell of an animal, fungus or protist, the chimeric DNA comprising

(a) a promoter or promoter region recognizable by RNA polymerases in the cell; operably linked to
(b) a DNA region which when transcribed yields a chimeric sense nucleic acid molecule as hereinbefore described.

The invention also provides a library of chimeric genes comprising multiple individual chimeric genes, each being different, wherein each individual chimeric gene encodes a chimeric nucleic acid molecule or comprises a chimeric DNA molecule as hereinbefore described.

A further aspect of the present invention provides a research reagent or kit comprising a nucleic acid vector for use in preparing a chimeric nucleic acid molecule or comprising a chimeric DNA molecule as hereinbefore described.

The invention also provides a package comprising the research reagent or kit described above and instructions for use thereof.

In a further aspect of the invention there is provided a composition comprising a chimeric nucleic acid molecule or a chimeric DNA molecule as hereinbefore described and a pharmaceutically acceptable carrier.

Another aspect of the invention provides a method of preparing a medicament for the treatment of an animal disease, comprising the composition of the invention.

The invention also provides a method of treating or preventing a disease in an animal, the method comprising administering a composition of the invention to an animal in need thereof.

A further aspect of the invention provides use of the composition of the invention in the preparation of a medicament for treating an animal disease.

In yet another aspect of the invention there is provided a method of identifying or characterising a nucleic acid-nuclear localization signal in an isolated nucleic acid molecule, comprising the steps of

(a) providing a first a cell with a first chimeric nucleic acid molecule wherein the molecule comprises
(i) a target-gene specific region comprising a nucleotide sequence of at least about 16 consecutive nucleotides having at least about 94% sequence identity with the complement of 16 consecutive nucleotides from the nucleotide sequence of transcribed nucleic acid sequence of the target gene,
wherein the target gene is a reporter gene, a pathogenic animal virus gene, a cancer-related gene, an oncogene, an immunomodulatory gene, a gene encoding a cytokine, growth factor, enzyme or a transcription factor, and
(ii) a largely double stranded nucleic acid region comprising a nucleotide sequence obtained from the isolated nucleic acid molecule; and
(b) providing a second cell with a second nucleic acid molecule, comprising the antisense region but not the largely double stranded nucleic acid region; and
(c) determining the extent of down-regulation of the target gene expression in the first cells in the presence of the first chimeric nucleic acid molecule and the second cells in the presence of the second nucleic acid molecule,
wherein the first cell and the second cell is of an animal, fungus or protist.

### BRIEF DESCRIPTION OF THE FIGURES

FIG. 1: shows a diagrammatic representation of a model of post-transcriptional gene silencing (PTGS), also known as RNA interference (RNAi). The introduction of a double stranded RNA (dsRNA) to a cell results in production of short interfering RNAs which may complex with cellular machinery for sequence-specific degradation of target RNAs.

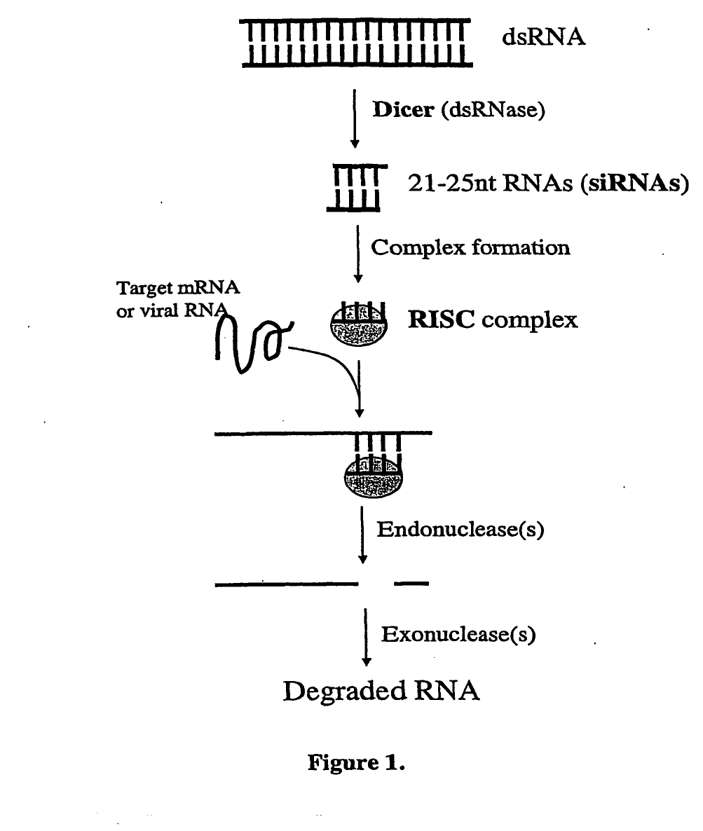

FIG. 2: shows production of a dsRNA from a transgene comprising an inverted repeat sequence may result in the production of siRNAs that are exported to the cytoplasm of the cell, where they cause degradation (PTGS) of target RNA.

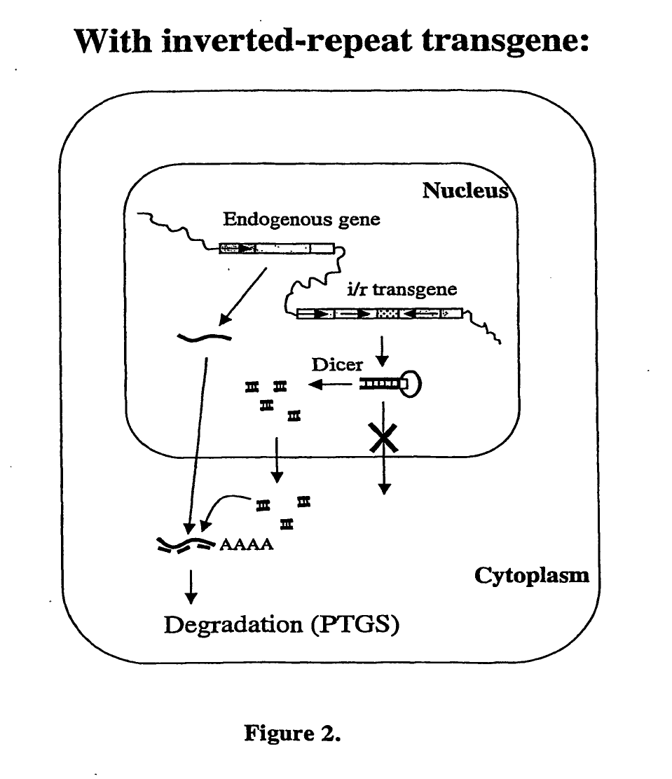

FIG. 3: shows antisense molecules produced in the nucleus by transcription of an antisense transgene may not be effective for gene silencing because they are exported to the cytoplasm where they may not result in production of siRNAs.

FIG. 4: shows a schematic representation of the secondary structure predicted using Mfold software for different viroids of the PSTVd-type. A. Potato spindle tuber viroid; B. Australian grapevine viroid; C. Coconut tinangaja viroid; D. Tomato planta macho viroid; E. Hop latent viroid of thermomutant T229; F. Tomato apical stunt viroid.

FIG. 5: shows nucleotide sequence comparison of the PSTVd sequences obtained and used. Upper sequence is for PSTVd clone 1-4 (mPSTVd) (SEQ ID NO: 11), lower sequence is for clone 1-9 (PSTVd) (SEQ ID NO: 10).

FIG. 6: shows a schematic representation of the predicted secondary structure of: pPSTVd region in clone 1-9 (and pMBW491 etc), adopting almost the wild type (strain RG1) rod-like configuration (upper structure); and of the mPSTVd region of clone 1-4 (in pMBW489 etc) where a nucleotide deletion results in a structure different from the wildtype configuration.

FIG. 7: shows a schematic representation of the various chimeric gene constructs used in Examples 1 and 2. CMV promoter: cytomegolovirus promoter; SV40 poly(A): transcription termination and polyadenylation region from SV40; PSTVd: Potato Spindle Tuber Viroid sequence; CUGrep: sequence comprising 54 repeats of the CUG sequence; humGFP: humanized green fluorescent protein coding region (adapted to the codon usage of human genes; the sense orientation of this region with respect to the promoter is indicated by the horizontal arrows); Pdk intron: Flaveria trinervia pyruvate orthophosphate dikinase 2 intron 2.

FIG. 8: shows a schematic representation of the construction of pMBW496, and the corresponding DNA sequence of the CUG repeat-encoding region (SEQ ID NO: 25). Abbreviations as for FIG. 7 legend.

FIG. 9: shows a schematic representation of the rod-like RNA structures formed when the exemplified RNA sequences fold, as predicted by MFOLD. Potential nuclear retention nucleic acid sequences for use in animal include viroid sequences such as, for example, PSTVd type viroids which form a rod-like structure or imperfect hairpin (upper panel), long trinucleotide repeats such as CUG repeats (lower panel) (SEQ ID NO: 26), Hepatitis delta RNA sequences or similar satellite RNA sequences that form a long hairpin, and synthetic hairpin sequences with frequent mismatches.

FIG. 10: shows a graphical representation of the level of GFP expression from pMBW450 in CHO cells, in the presence of increasing amounts of the test plasmid pMBW449 (“asGFP”) (upper panel) or pMBW491 (“asGFP-PSTVd”) (lower panel).

FIG. 11 shows a graphical representation of the level of GFP expression from pMBW450 in CHO cells, in the presence of increasing amounts of the test plasmid pMBW489 (“asGFP-mPSTVd”) (upper panel) or pMBW496 (“asGFP-CUGrep”) (lower panel).

FIG. 12: shows a comparison of the GFP expression level in CHO cells in the presence of different effector plasmids, each at 0.3 μg per cell aliquot, and of the target gene construct.

FIG. 13: shows a graphical representation of the level of GFP expression from pMBW450 in HT29 (cancer) cells, in the presence of increasing amounts of the test plasmid pMBW449 (“asGFP”) (upper panel) or pMBW491 (“asGFP-PSTVd”) (lower panel).

FIG. 14: shows a graphical representation of the level of GFP expression from pMBW450 in HT29 cells, in the presence of increasing amounts of the test plasmid pMBW496 (“asGFP-CUGrep”) (upper panel) or pLMW92 (“hairpin RNA”) (lower panel).

FIG. 15: shows a graphical representation of the level of GFP expression from pMBW450 in HT29 cells, in the presence of increasing amounts of the test plasmid pLMW93 (“asGFP-asGFP”).

FIG. 16: shows the RNA sequence of human U6 snRNA (SEQ ID NO: 27).

FIG. 17: shows a diagrammatic representation of the Folding of human U6 RNA sequence-MFOLD output.

FIG. 18: shows a diagrammatic representation of the gene silencing constructs tested in animal cells as described in Example 3.

FIG. 19: shows graphs indicating gene silencing in HeLa cells, 48 hours post-transfection. The graphs show representative fluorescence for each gene silencing (NTS) construct indicated in FIG. 18. The numbers inside each histogram are mean fluorescence intensity±standard error. TA only is the transfection agent (TA) alone cell control.

FIG. 20: shows a graph indicating EGFP intensity obtained in the presence of gene silencing constructs indicated in FIG. 18 relative to that obtained in the presence of pMBW497 (100%), using the FACS data of FIG. 19.

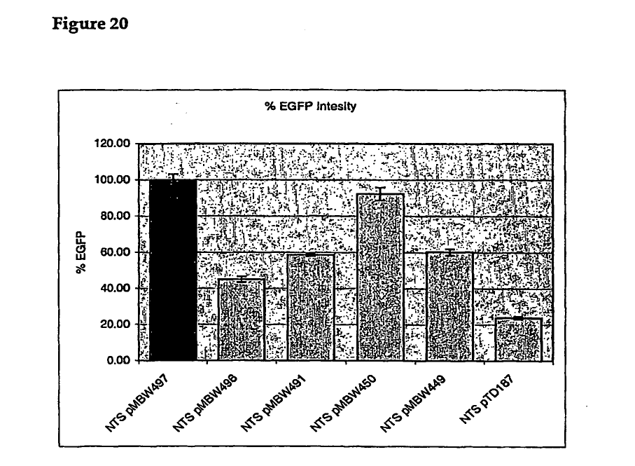

FIG. 21: shows a diagrammatic representation of gene silencing plasmids for Influenza A NP gene.

### DETAILED DESCRIPTION OF THE INVENTION

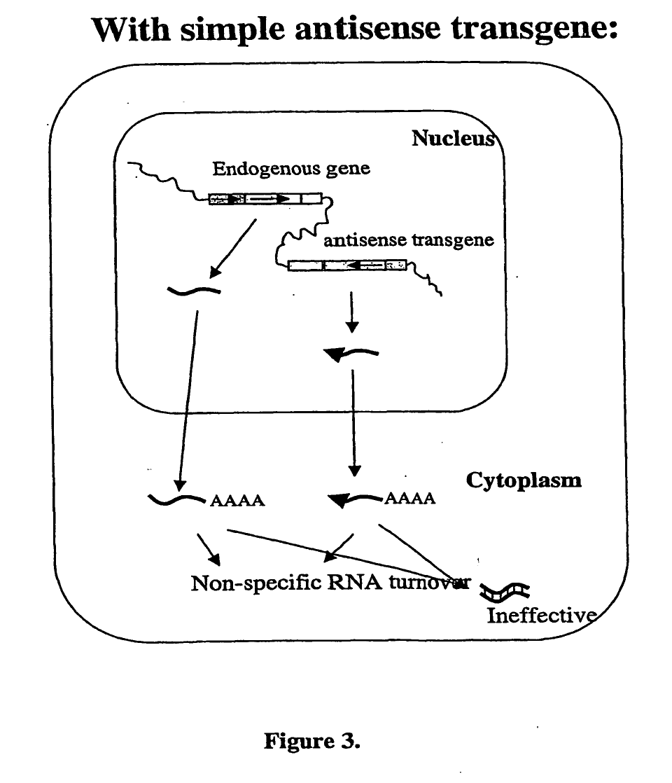

The presently described methods and means for obtaining enhanced nucleic acid-mediated down regulation of gene expression, in a cell of an animal, fungus or protist, are based upon the unexpected observation that a chimeric nucleic acid molecule comprising a target gene-specific nucleic acid sequence in combination with a largely double stranded nucleic acid region which preferably comprises a nuclear localization signal increases the efficiency of the target gene down-regulation. The double-stranded nucleic acid region must be largely, but not entirely, double-stranded and thereby itself does not induce down-regulation of its own sequence or non-specific interferon responses in the cell. There are numerous reported observations that many antisense nucleic acids, particularly if expressed from a transgene in the nucleus of a cell, are not optimally effective for gene silencing a target RNA. This might be a consequence of the cytoplasmic localization of such antisense nucleic acids (FIG. 3).

Thus, in one aspect of the invention there is provided a method of down regulating the expression of a target gene in a cell of an animal, fungus or protist, the method comprising the step of providing the cell with a chimeric nucleic acid molecule, wherein the molecule comprises

a) a target-gene specific region comprising a nucleotide sequence of at least about 16 consecutive nucleotides having at least about 94% sequence identity with the complement of 16 consecutive nucleotides from a transcribed nucleotide sequence of the target gene, and
b) a largely double stranded nucleic acid region,
wherein the target gene is a reporter gene, a pathogenic animal virus gene, a cancer-related gene, an oncogene, an immunomodulatory gene, a gene encoding a cytokine, growth factor, enzyme or a transcription factor or an animal disease causing gene.

The phrase “down regulating the expression of a target gene” as used herein is taken to mean that production of a polypeptide of a target gene or nucleic acid of interest in a cell is decreased or prevented as compared to the expression of the target gene or nucleic acid prior to treatment of the cell. The term “gene expression” or “expression of a nucleic acid” is used herein to refer to the process wherein a gene or nucleic acid is transcribed (or replicated) to yield a RNA copy of all or part of the gene or nucleic acid, and optionally translated to yield a polypeptide or protein.

In the present invention the expression of a target gene or nucleic acid of interest in a cell in the presence of a chimeric nucleic acid molecule of the invention is down regulated compared to the expression of the target gene in the absence of the chimeric nucleic acid molecules of the invention.

The expression of the target gene in the presence of the chimeric nucleic acid molecule of the invention should thus be lower than the expression in the absence thereof, at least for some time in at least some of the cells treated. The extent of the reduction of gene expression may be at least about 50% or 75% or 90% or preferably at least about 95% of the level of phenotypic expression in the absence of the chimeric nucleic acid molecule. For some applications, the expression may be inhibited by the presence of the chimeric nucleic acid molecule or the chimeric gene encoding such a nucleic acid, to the extent that expression is not detected. The extent of the reduction of gene expression may be measured by any of the methods known in the art, including nucleic acid hybridisation, for example Northern or slot blotting or RNAse protection assays or microarray analysis, or reverse transcription-PCR (RT-PCR) or through measuring the reduction of the protein product encoded by the gene, for example through enzymatic assay or immunological detection such as ELISA or Western blot assay, or through some other phenotype associated with the reduced gene expression.

The “target gene” as used herein is taken to refer to any nucleic acid of interest which is present in a cell of an animal, fungus or protist. The target gene may be transcribed into a biologically active RNA or it may be part of a larger RNA molecule of which other parts are transcribed into a biologically active RNA. The target gene may be an endogenous gene, it may be a transgene that was introduced through human intervention in the ancestors of the cell, or it may be a gene introduced into the cell by an infectious or pathogenic organism. The target gene may also be of viral origin.

Furthermore, the sequence of at least 16 nucleotides that is targeted by the chimeric nucleic acid molecule may be selected from translated or non-translated regions or intron or preferably exon regions, that is, the coding region, or the 5′UTR or 3′UTR, or a combination of any or all of these.

The term “targeting” as used herein describes an interaction between the chimeric nucleic acid of the invention and a target nucleic acid. Such interaction may be based on hybridisation using hydrogen bonding, which may be Watson-Crick, Hoogsteen or reversed Hoogsteen hydrogen bonding between essentially complementary nucleoside or nucleotide bases. It will be apparent to those skilled in the art that this complementarity is not necessarily complete along the full length of the target gene specific region. Rather, the degree of complementarity must be such as to allow, under physiological conditions, stable and specific binding between the target gene specific region and the target nucleic acid. Specific interaction or targeting may be realised upon binding of the chimeric nucleic acid molecule to the target nucleic acid and the consequent down regulation of the normal expression or function of the target nucleic acid. A sufficient degree of complementarity is desired to avoid non-specific binding of the compound to non-target nucleic acid sequences. The physiological conditions include, for example, the conditions in the cell or organism or similar conditions in vitro.

The target gene may be any gene which is expressed in a cell of an animal, fungus or protist. In an embodiment, the target gene is a reporter gene, a pathogenic animal virus gene, a cancer-related gene, an oncogene, an immunomodulatory gene, a gene encoding a cytokine, growth factor, enzyme or a transcription factor or an animal disease causing gene. An “animal disease causing gene” includes any gene which is involved in an animal disease or condition wherein reduction of expression of that gene results in a reduction, delay or prevention of a disease or condition in the animal. In an embodiment, the disease is a human disease or a disease of a domestic animal, such as dogs, cats, horses and farm animal, such as cows, sheep, pig and goats.

Immunomodulatory genes includes any genes involved in or controlling the immune system of a vertebrate animal such as, for example, humans, wherein down regulation of such genes in a cell alters the function of the immune system in an animal expressing such genes.

A reporter gene refers to a specific gene that is inserted into the DNA of a cell so that cell will “report” (to researchers, clinicians) when signal transduction has occurred in that cell, or when a (linked) gene was successfully expressed in the cell. For example, a suitable reporter gene can include the enzyme luciferase (which catalyzes bioluminescence—light production) or more preferably a gene encoding green florescent protein (GFP). Another preferred reporter gene is an enhanced green florescent protein (EGFP).

The target gene used in the present invention may cause a disease in an organism or be involved in causing the disease and is a gene where reduction of the particular gene expression is required to prevent or alleviate the disease. The biological processes affected by the disease that may be reversed by down-regulation of the specific gene target include cell proliferation, cell migration or metastasis, apoptosis, stress signalling, and cell attachment. The target gene(s) may encode enzymes, transcription factors, cytokines, growth factors, cell adhesion or motility factors, cell cycle factors, tumour suppressors, or cell cycle inhibitors.

The target gene may be a gene from a pathogenic animal virus, for example human immunodeficiency virus (HIV), herpes simplex virus-1 (HSV-I), HSV-2, cytomegalovirus (CMV), a hepatitis virus such as hepatitis B, hepatitis C or hepatitis D viruses, papillomaviruses, RNA viruses such as polio viruses, VSV, Influenza virus, morbillivirus, or a double-stranded RNA virus such as a reovirus. The virus may be pathogenic to animals other than humans, for example Foot and Mouth Virus, Rinderpest virus, Blue tongue virus, Swine Fever virus, Porcine circa virus, Capripox virus, West Nile Virus, Henipah virus, Marek's Disease Virus, Chicken Aneamia Virus, Newcastle Disease Virus, Avian Influenza virus, Infectious Bursal Disease Virus, Aquaculture viruses such as iridoviruses, paramyxoviruses or White Spot Syndrome Virus.

The target gene may preferably be a gene from HIV. HIV causes acquired immune deficiency syndrome (AIDS) in humans, characterised by progressive loss of CD4+ T lymphocytes, monocytes and macrophages and an associated impairment of immune function, often including the presence of opportunistic infections and neurological complications. HIV is a member of the lentivirus subfamily of retroviruses and includes HIV-1, the predominant type, and HIV-2 found primarily in Africa and India. Each virion comprises two strands of RNA and several proteins including spike (envelope) and capsid proteins as well as other proteins such as integrase. Like other retroviruses, the RNA is reverse transcribed into a DNA copy that moves into the nucleus and is integrated into the host cell DNA. Expression of the integrated provirus involves transcription initiating from the long terminal repeat (LTR), splicing of the transcript and translation to form new viral proteins. The HIV genes include 5′LTR, gag, pol, vif, vpr, tat, rev, env, nef and 3′LTR. Gag encodes the core protein or capsid protein. Pol encodes reverse transcriptase, protease, ribonuclease and integrase which are required for integration. Env encodes the two envelope or spike proteins gp120 and gp41. The tat gene encodes a regulatory protein that activates transcription of the HIV provirus, while rev regulates processing and/or export of viral transcripts. Any of these HIV genes are suitable for targeting by the chimeric nucleic acids of the present invention. Nucleotide sequences for numerous HIV isolates have been obtained and are available at the following web site: hiv-web.lanl.gov. Since HIV has a high mutation rate and multiple strains can be present in an infected patient, it is preferred that conserved nucleotide sequences of the virus are targeted by the chimeric nucleic acid molecules. For example, sequences 5831-5849 (ATGGAGCCAGTAGATCCTA) (SEQ ID NO: 12), 5852-5870 (CTAGAGCCCTGGAAGCATC) (SEQ ID NO: 13), and 5971-5989 (TGGCAGGAAGAAGCGGAGA) (SEQ ID NO: 14) within the tat gene of strain HXB2 are highly conserved in the corresponding regions in other strains. Such comparisons can readily be made using the service at the following web site: hivweb.lanl.gov. Other useful sequences in the HIV genomes can be readily identified in a similar fashion. HIV infection may also be reduced by down-regulation of the genes encoding the CXCR4 and CCR5 coreceptors, members of chemokine receptor subfamilies that are required for entry of HIV-1 into cells.

The target gene may preferably be a gene of a hepatitis virus. Hepatitis viruses include hepatitis B virus, hepatitis C virus and hepatitis D virus. Hepatitis B virus (HBV) is a member of the hepadna virus family and is a small, enveloped, partially double-stranded DNA virus with a circular genome size of about 3.2 kb. HBV replicates its genome by reverse transcription and integration into the host cell DNA. Integration of the viral DNA is not necessary for viral replication but does allow persistence of the viral genome in the cell, and often precedes the induction of hepatocellular carcinoma. The coding minus strand has four open reading frames that overlap in part and that encode at least seven proteins including core protein, HBeAg, HBsAg, a DNA polymerase with reverse transcriptase activity, and a transcriptional transactivator encoded by the X gene which is required for infection of liver cells in vivo. The HBx gene is highly conserved amongst hepadnaviruses. HBV strains can be classified into at least seven genotypes having 85-90% nucleotide sequence identity between the genotypes. HBV is transmitted by exposure to infected blood or other body fluids, for example through blood transfusion, intravenous drug use or perinatally. Numerous HBV nucleotide sequences are available from publicly available databases such as at the websites ncbi.nlm.nih.gov or s2 as02.genes.nig.ac.ip/index.html. Comparison of nucleotide sequences reveals highly conserved regions which are preferred for targeting with the chimeric nucleic acid molecules and methods of the invention, for example see McCaffrey et al, Nature Biotechnology 21:639-744 (2003) who targeted seven conserved sequences of HBV with dsRNA, herein incorporated by reference.

Hepatitis C virus (HCV) is a RNA virus belonging to the Flavivirus family which also includes yellow fever, dengue and Japanese B encephalitis viruses. The genome is a single stranded RNA of about 9.4 kb which can be directly targeted by the chimeric nucleic acids of the invention. There is no evidence that the HCV RNA integrates into the host genome. The genome encodes several proteins including core, E2, NS3 (protease), NS4B, NS5A (RNA polymerase), NS5B and helicase proteins. Any of the HCV genes can be targeted, for example the gene encoding non-structural protein 5B (NS5B)-viral polymerase was targeted by dsRNA (McCaffrey et al., Nature 418:38-39 (2002)) and can similarly be targeted by the molecules of the present invention. Analysis of the thousands of HCV nucleotide sequences available shows that different isolates vary in nucleotide sequence throughout the viral genome, however, more conserved regions can readily be identified. Preferred regions of the HCV genome that can be targeted include the 5′UTR of about 341 nucleotides (Han et al., Proc Natl Acad Sci USA 88:1711-1715 (1991)), 3′UTR, preferably the 5′ hairpin loop region or the R2 region, even more preferably the translation initiation codon region, for example the region of nucleotides 330-349. In another embodiment, the region comprising nucleotides 1-686 comprising the entire 5′-untranslated region (nucleotides 1-341) and a 145-nucleotide core region sequence of HCV RNA can be targeted. HCV isolates can be grouped in at least six genotypes that show different geographic distributions, with genotype 1 most common in North America and Western Europe. Numerous HCV nucleotide sequences are available from publicly available databases such as at the websites or s2 as02.genes.nig.ac.ip/index.html.

Hepatitis D virus (HDV) is also regarded as a virusoid because it requires the surface coat of HBV in order to be infectious, so is always associated with HBV infection. The genome encodes p24 and p27 viral polypeptides and an ORFS polypeptide. U.S. Pat. No. 5,932,219 discloses the entire genome of a hepatitis D virus and cDNA sequences from HDV.

The target gene may be a cancer causing gene, such as genes required or responsible for the development or growth or spread of cancers. The cancer may be breast cancer, lung cancer, liver cancer, colon cancer, pancreatic cancer, prostate cancer, glioblastoma, or leukaemia. Examples of cancer-related genes that may be targeted are: oncogenes including genes encoding nuclear oncoproteins or cytoplasmic/membrane-associated oncoproteins, genes encoding cellular receptors, cytokines, growth factors, inhibitors of tumour suppressor genes. Genes encoding oncoproteins include c-myc, N-myc, c-myb, c-fos, c-fos/jun, PCNA, p120; EJ-ras, c-Ha-ras, N-ras, rrg, bcl-2, cdc-2, c-raf-1, c-mos, c-src, c-abl, Bcr-Abl, c-ets, telomerase, cyclins, cyclin dependent kinases; cellular receptors include, for example, EGF receptor, Her-2, c-erbA, VEGF receptor (KDR-1), retinoid receptors, protein kinase regulatory subunit, c-fms, Tie-2, c-raf-1 kinase, PKC-alpha, protein kinase A (R1 alpha); cytokines or growth factors include, for example, CSF-1, IL-6, IL-1a, IL-1b, IL-2, IL-4, IL-6, IL-8, bFGF, VEGF, myeloblastin, fibronectin; inhibitors of tumor suppressor genes such as, for example, MDM-2.

The target gene may encode the Bcl-2 group of proteins including Bcl-2, Bcl-xL, Mcl-1 and A1. These genes encode proteins that are apoptosis inhibitors and are overexpressed in numerous cancers including some lymphomas and leukemias. They are also implicated in resistance to cancer treatment involving apoptosis. Antisense oligonucleotides targeting the Bcl-2 transcript have proven effective in promoting apoptosis of certain cancer cells, particularly in combination with other anti-cancer agents. It is preferred to target regions of the gene transcripts including the translation initiation codon. Protein kinase C-α proteins are a family of serine/threonine kinases involved in signal transduction pathways for responses arising from G-protein coupled receptors, and deregulation of PKC-α has been implicated in the deregulation of cell growth and tumour development. Gene transcripts encoding PKC-α may be targeted. Raf kinase encodes a serine-threonine kinase that is activated by the Ras protein and may also regulate apoptosis. Mutations of the raf or ras genes resulting in overexpression or constitutive expression have been identified in many cancers. Preferred target sites in the transcripts of these genes are the regions including the translation initiation codons or the 3′UTRs. Protein kinase A RI-α overexpression is associated with cell proliferation and neoplastic transformation, and antisense-mediated down regulation of the gene encoding PKA RI-α has been shown to be effective in inhibiting the growth of tumour cell lines in vitro. Other cancer-related genes that may be targeted by the molecules of the invention include genes encoding testosterone-repressed prostate message-2 (clusterin) and inhibitors of apoptosis (IAP) such as X-linked IAP, cIAP1, cIAP2, NAIP and Survivin, Her-2/neu, insulin-like growth factor-1 (IGF-1) and other growth factor receptors.

The nucleic acid molecules of the present invention may be suitable, for example, for the treatment of disorders which are influenced by integrins, or cell-cell adhesion receptors, for example by VLA-4, VLA-2, ICAM, VCAM or ELAM.

The chimeric nucleic acid molecules of the present invention may be directed against gene targets responsible for cell proliferation or migration. The molecules may be suitable, for example, for preventing restenosis. Examples of such gene targets are: genes encoding nuclear transactivator proteins and cyclins such as, for example, c-myc, c-myb, c-fos, c-fos/jun, cyclins and cdc2 kinase, genes encoding mitogens or growth factors such as, for example, PDGF, bFGF, VEGF, EGF, HB-EGF and TGF-beta, genes encoding cellular receptors such as, for example, bFGF receptor, EGF receptor and PDGF receptor. In certain embodiments, the methods and molecules described herein can be employed for the treatment of autoimmune disorders, for example by inhibiting expression of genes which encode or regulate the expression of cytokines. Accordingly, chimeric nucleic acid molecules that down regulate expression of cytokines such as THF, IL-1, IL-6 or IL-12, or a combination thereof, can be used as part of a treatment or prophylaxis for rheumatoid arthritis. Similarly, chimeric nucleic acid molecules that down regulate expression of cytokines involved in inflammation can be used in the treatment or prophylaxis of inflammation and inflammation-related diseases, such as multiple sclerosis, for example by inhibition of VLA4, or for the treatment of asthma by down regulating expression of the adenosine-A1 receptor, adenosine-A3 receptor, Bradikinin receptor or of IL-13.

The target gene may also be involved in cardiovascular diseases, for example, genes encoding a β1-adrenergic receptor, angiotensin type 1 (AT1) receptors, angiotensinogen (ATG), angiotensin converting enzyme (ACE), a protein that negatively regulates the activity of the NF-B transcription factor, C-reactive protein (CRP), EGF receptor kinase, heparin binding EGF (HB-EGF), TGFβ, VEGF, FGF-4 or a protein from the EDG family such as, for example, Edg-1. For instance, in the treatment of myocardial infarction, chimeric nucleic acid constructs may be provided to promote angiogenesis and thereby promote recovery or prevent further damage to the tissue in an around the infarct. Intrapericardial delivery can be used for delivery of the chimeric nucleic acids to reduce proliferation or migration of smooth muscle cells and thereby may be useful in treating neointimal hyperplasia, such as restenosis, artherosclerosis and the like. For example, the chimeric nucleic acids can be used for down regulating gene expression of c-myb, c-myc, proliferating cell nuclear antigen (PCNA), transforming growth factor-beta (TGF-beta), or transcription factors such as nuclear factor kappaB (NF-B) and E2F. The chimeric nucleic acids can also be delivered in a localised fashion on coated stents, either by directly coating at least a portion of the stent or through a polymeric coating from which the chimeric nucleic acids are released.

The target gene may also be a gene associated with diabetes, for example the PTP-1B gene.

In the present invention a cell is of an animal, fungus or protist. The cell can be of an animal, including but not limited to, a mammal, reptile, amphibian, fish or bird. In an embodiment, the animal is a vertrabrate, more preferably, a mammal, and most preferably a human.

The invention is also applicable to fungal cells. The term “fungus” is taken to mean any organism that is a saprophytic and parasitic plant that lacks chlorophyll and flowers, including but not limited to, molds, toadstools, rusts, mildews, smuts, ergot, mushrooms Aqaricus bisporus and yeasts.

The invention is also useful for down regulation of gene expression in cells or organisms which are fungi, for example Neurospora crassa and Ascobolus immerses which are filamentous fungi where post-transcriptional gene silencing has been observed (Cogoni, Ann Rev Microbiol 55:381-406 (2001)) and yeasts. Any fungal genes may be down regulated including those encoding enzymes or transcription factors. PTGS in fungi, also termed “quelling” has been observed for genes such as al-1 and al-3 which are required for carotenoid biosynthesis (Romano and Macino, Mol Microbiol 6:3343-3353 (1992)), hph (Pandit and Russo, Mol Gen Genet 234:412-422 (1992)), we-1 (Ballario et al., EMBO J 15:1650-1657 (1996)), wc-2 (Linden and Macino, EMBO J 16:98-109 (1997)) and ad-9, and appears to be a general phenomenon in fungi. It has also been found that genes involved in PTGS in fungi, animals and plants are highly conserved, such as the genes qde-1, qde-2 and qde-3 from N. crassa or genes encoding Dicer, pointing to evolutionarily conserved mechanisms. PTGS has also been observed in invertebrate animals such as planaria (Sanchez et al., Proc Natl Acad Sci USA 96:5049-5054 (1999)), and hydra (Lohmann et al., Dev Biol 214:211-214 (1999)) and protozoa such as trypanosomes, for example Trypanosoma brucei (Ngo et al., Proc Natl Acad Sci USA 95:14687-14692 (1998); Shi et al., RNA 6:1069-1076 (2000)) or Plasmodium falciparum (McRobert and McConkey, Molec Biochem Parasitol 119:273-278 (2002)). The present invention is particualry useful for targetting fungal genes that are involved in fungal diseases of organisms or fungal genes that are vital for survival of fungal cells.

The term “protists” as used herein is taken to mean a microscopic, single-celled animal form. For instance, flagellate protozoa are protists that include the family Trypanosomatidae which includes various members of the genera Leishmania and Trypanosoma, including unicellular protozoal pathogens. Expression of any protozoal target gene can be down regulated by PTGS, for example telomerase-associated protein p43 in the ciliated protozoa Euplotes (Mollenbeck et al., J Cell Sci 116:1757-1761 (2003)). There are reports of PTGS in molds such as Dictyostelium discoideum (Martens et al., Mol Biol Cell 13:445-453 (2002)) and in unicellular green algae such as Chlamydomonas reinhardtii (Wu-Scharf et al. Science 290:1159-1162 (2000)). The cells or organisms include any from the kingdom Protista (protists), which include unicellular, colonial and multicellular eukaryotes that do not have the distinctive characters of animals, plants or fungi. The protista include the phyla Myxomycota (plasmodial slime molds), Oomycota (commonly called water molds) and Chlorophyta (green algae). Therefore, the present invention is also useful for application to protists.

In the present invention a target gene can be down regulated in an in vivo cell or an in vitro cell. The cell may be a primary cell or a cell that has been cultured for a period of time or the cells may be comprised of a cultured cell line. The cell may be a disesead cell, such a cancer cell or tumor or a cell infected by a virus. The cell may be a stem cell which gives rise to progenitor cells, more mature, and fully mature cells of all the hematopoietic cell lineages, a progenitor cell which gives rise to mature cells of all the hematopoietic cell lineages, a committed progenitor cell which gives rise to a specific hematopoietic lineage, a T lymphocyte progenitor cell, an immature T lymphocyte, a mature T lymphocyte, a myeloid progenitor cell, or a monocyte/macrophage cell. The cell may be a stem cell or embryonic stem cell that is omnipotent or totipotent. In an embodiment, the cell is omnipotent. The cell may be a a nerve cell, neural cell, epithelial cell, muscle cell, cardiac cell, Liver cell, kidney cell, stem cell, embryonic or foetal stem cell or fertilised egg cell.

The term “nucleic acid” as used herein refers to any polymer of nucleotides, which may be a single molecule or more than one molecule linked by non-covalent bonds and may be double stranded or partly single-stranded and partly double stranded. A “region” or “portion” of a nucleic acid molecule refers to a set of linked nucleotides which is less than the entire molecule.

The terms “chimeric gene” or “chimeric nucleic acid” as used herein, refers to a gene or nucleic acid, which is not found in nature in the cell of interest or, alternatively, any gene or nucleic acid comprising at least one element which is not associated in nature with another part or the remainder of the gene or chimeric nucleic acid. The nucleic acid may comprise RNA, comprised of ribonucleotides, or DNA, comprised of deoxyribonucleotides, or a combination of these and optionally may comprise non-nucleotide components. The nucleic acid is preferably RNA.

As used herein “comprising” is to be interpreted as specifying the presence of the stated features, integers, steps or components as referred to, but does not preclude the presence or addition of one or more features, integers, steps or components, or groups thereof. Thus, e.g., a nucleic acid or protein comprising a sequence of nucleotides or amino acids, may comprise more nucleotides or amino acids than the actually cited ones, i.e., be embedded in a larger nucleic acid or protein. A chimeric gene comprising a DNA region which is functionally or structurally defined, may comprise additional DNA regions etc.

It will thus be clear that the region of the chimeric nucleic acid molecule, of at least 16 nucleotides which is identical or nearly identical in sequence to the target-gene specific region may be comprised within a larger nucleic acid molecule, varying in size from 16 nt to a length equal to the size of the transcript of the target gene with a varying overall degree of sequence identity.

For the purpose of this invention, the “sequence identity” of two related nucleotide or amino acid sequences, expressed as a percentage, refers to the number of positions in the two optimally aligned sequences which have identical residues (×100) divided by the number of positions compared. A gap, i.e., a position in an alignment where a residue is present in one sequence but not in the other is regarded as a position with non-identical residues. The alignment of the two sequences is performed by the Needleman and Wunsch algorithm (Needleman and Wunsch 1970). The computer-assisted sequence alignment above, can be conveniently performed using standard software program such as GAP which is part of the Wisconsin Package Version 10.1 (Genetics Computer Group, Madision, Wis., USA) using the default scoring matrix with a gap creation penalty of 50 and a gap extension penalty of 3. Sequences are indicated as “essentially similar” when such sequence have a sequence identity of at least about 75%, particularly at least about 80%, more particularly at least about 85%, quite particularly about 90%, especially about 95%, more especially about 100%, quite especially are identical. It is clear that when RNA sequences are to be essentially similar or have a certain degree of sequence identity with DNA sequences, thymine (T) in the DNA sequence is considered equal to uracil (U) in the RNA sequence. Thus when it is stated in this application that a sequence of 16 consecutive nucleotides has a 94% sequence identity to a sequence of 16 nucleotides, this means that at least 15 of the 16 nucleotides of the first sequence are identical to 15 of the 16 nucleotides of the second sequence.

The mentioned target-gene specific nucleotide regions may thus be at least 16 nucleotides (nt), 19 nt, 21 nt, 19-25 nt, 50 nt, 100 nt, 200 nt, 300 nt, 500 nt, 1000 nt, 2000 nt or even about 5000 nt or larger in length, each having an overall sequence identity of about 40% or 50% or 60% or 70% or 80% or 90%, 94% or 100% to the complement of the target nucleotide sequence. The longer the sequence, the less stringent the requirement for the overall sequence identity is.

Furthermore, multiple sequences with sequence identity to the complement of transcribed nucleotide sequence of multiple target-gene specific nucleic acid regions may be present within one chimeric nucleic acid molecule. That is, the chimeric nucleic acid molecule may comprise two, three, four or up to at least 10 nucleotide sequences, each having sequence identity to the complement of the nucleotide sequence of the transcribed target RNA, each of which may be the same or different. At least one of the nucleotide sequences of the chimeric nucleic acid molecule comprises at least 16 consecutive nucleotides having at least about 94% sequence identity with the complement of 16 consecutive nucleotides of a transcribed nucleotide sequence of the target gene, and preferably at least two of the nucleotide sequences have at least 16 consecutive nucleotides with at least about 94% sequence identity with the complement of the target transcript. Also, multiple sequences with sequence identity to the complement of transcribed nucleotide sequence of several target genes may be present within one chimeric nucleic acid molecule. That is, the chimeric nucleic acid molecule may target transcripts of two or more genes. Such multiple sequences within the one chimeric nucleic acid molecule may directly concatenated or may be joined by linker or spacer regions which preferably comprise nucleotides. The nucleotide linkers may also comprise nucleotides which form stem-loop structures that serve to increase the likelihood of maintaining the target-gene specific regions in the single-stranded form and more accessible to the target RNA or increase stability in the cell.

The term “target-gene specific” as used herein is not to be interpreted in the sense that the chimeric nucleic acids according to the invention can only be used for down-regulation of that specific target gene. Indeed, when sufficient homology exists between the target gene specific RNA region and another gene, or when other genes share the same stretch of at least 16 nucleotides (such as genes belonging to a so-called gene-family), expression of those other genes may also be down-regulated.

The chimeric nucleic acid molecule of the invention comprises a largely double stranded nucleic acid region. As used herein, a “largely double stranded nucleic acid region” refers to a nucleic acid sequence, preferably comprising RNA or more preferably consisting of RNA, which is capable of folding into a rod-like structure by internal base-pairing and wherein the resulting rod-like structure does not comprise any stretch of 19 consecutive nucleotides having at least 94% sequence identity to the complement of another stretch of 19 other consecutive nucleotides within that nucleic acid molecule, which are capable of forming a double stranded region when the chimeric nucleic acid molecule comprising the largely double stranded nucleic acid region folds into a rod-like structure. In other words, the largely double stranded nucleic acid region upon folding does not contain a double stranded region of at least 19 by with at most one mismatch in those 19 bp, at least not in the energetically most favourable rod-like confirmation.

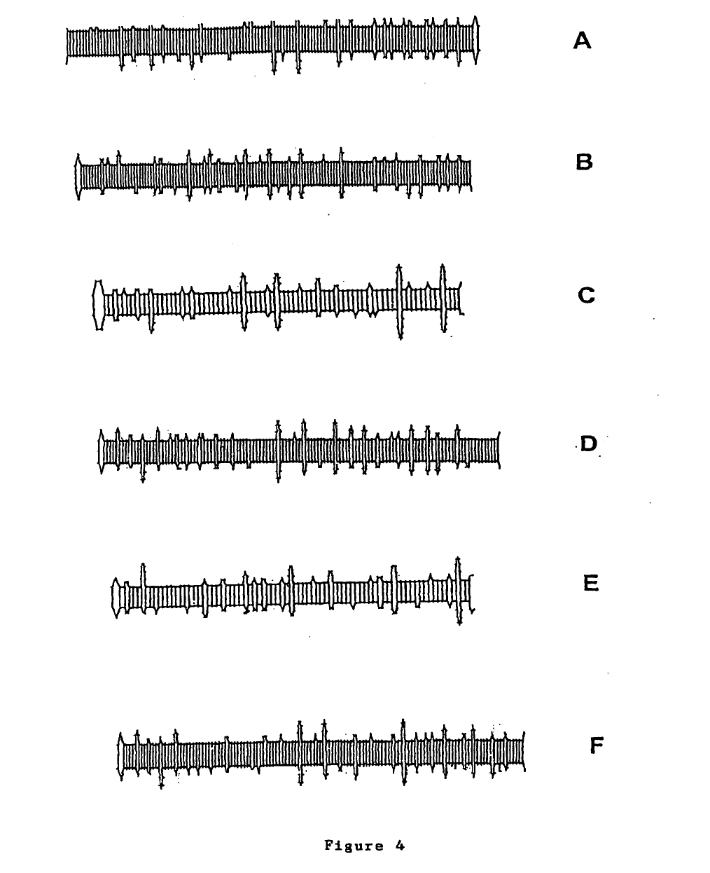

The largely double stranded nucleic acid region comprises two or more mismatched or non-basepaired nucleotides in each and every 19 nucleotide portion of each nucleotide strand that forms the double stranded region on folding. This can also be described by saying that the largely double-stranded nucleic acid region comprises double stranded nucleotide sequences each having 4-17 basepairs (A basepaired to T or U, G to C or U) with non-basepaired or mismatched nucleotides at both ends of the double stranded nucleotide sequences. Thus, the largely double stranded nucleic acid region is largely, but not completely, double stranded. The percentage of nucleotides within the largely double stranded nucleic acid region that are basepaired to other nucleotides within the largely double stranded nucleic acid region is preferably in the range 60-95%, more preferably 65-90% and most preferably 65-80%. The largely double stranded nucleic acid region may comprise at least 60 nucleotides, preferably at least 80, 100, 120 or 150 nucleotides, up to 300, 360 or more nucleotides. In an embodiment of the invention, the largely double stranded nucleic acid region consists of 100-360 ribonucleotides. This arrangement of mismatched or non-basepaired nucleotides in the largely double stranded nucleic acid region is intended to prevent or minimise the activation of post-transcriptional gene silencing responses to the chimeric nucleic acid molecule. Non-limiting examples of such structures are represented in FIG. 4.

Base-pairing of nucleotides as defined herein, unless otherwise stated, refers to standard Watson-Crick basepairing (G pairing to C, A pairing to U or T) or Hoogsteen or reversed Hoogsteen hydrogen bonding between essentially complementary nucleoside or nucleotide bases.

Although not intending to limit the invention to a specific mode of action, it is thought that such largely double stranded nucleic acid regions are involved in the nuclear localization of the chimeric nucleic acid molecules of which they are part. As a consequence thereof, the concentration of the target-gene specific regions in the nucleus may be increased, allowing a more efficient formation of sequence specific double-stranded nucleic acid formation, particularly dsRNA, by base pairing with the target gene transcript RNA.

As used herein, the term “capable of folding into a rod-like structure” with regard to a nucleic acid molecule refers to a secondary structure which the molecule will preferably adapt by internal basepairing and which has the overall appearance of a long rod. The rod-like structure may comprise branches or bulges (where non-matching nucleotides bulge out from the overall structure) and may be part of a larger secondary structure (which may or may not be rod-like). Examples of nucleic acid molecules capable of folding into a rod-like structure are represented in FIG. 4 and Bussière et al., Nucl Acids Res 10:1793-1798 (1996). The specific secondary structure adapted will be determined by the free energy of the nucleic acid molecule, and can be predicted for different situations using appropriate software such as FOLDRNA (Zuker and Stiegler, 1981) or the MFOLD structure prediction package of GCG (Genetics Computing Group; Zuker 1989, Science 244, 48-52).

In contrast to the largely double stranded region, the target-gene specific region of the chimeric nucleic acid molecule is preferably largely single stranded, that is, the majority of nucleotides are not basepaired to other nucleotides in the molecule.

The largely double stranded nucleic acid region preferably comprises a nuclear localization signal. The largely double stranded nucleic acid region may comprise a nucleotide sequence obtained from a viroid of the Potato Spindle Tuber Viroid (PSTVd)-type, a nucleotide sequence comprising at least 35 repeats of a trinucleotide CUG, CAG, GAC or GUC, a nucleotide sequence obtained from hepatitis delta RNA, or a synthetic nucleotide sequence comprising a nucleic acid-nuclear localization signal.

As used herein “nuclear localization” refers to a preferential localization of the chimeric nucleic acid molecule of interest in the nucleus of the cell compared to the cytoplasm of the cell. In the context of provision of the chimeric nucleic acid molecule of interest to the nucleus of the cell, for example by transcription in the nucleus of a gene encoding the chimeric nucleic acid molecule, nuclear localization may refer simply to preferential nuclear retention of the chimeric nucleic acid molecule. The extent of nuclear localization may be partial or preferably complete, where the chimeric nucleic acid molecule is detectable only in the nucleus of the cell. It will be appreciated that the preferential nuclear localization or nuclear retention is a property of the molecule as a whole but depends on the presence in the molecule of the largely double stranded nucleic acid region, comprising a “nuclear localization signal”.

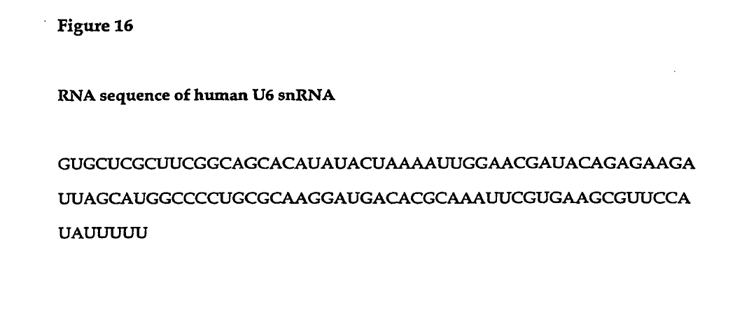

The largely double stranded nucleic region preferably comprises a nucleotide sequence obtained from a small nuclear RNA (snRNA). In an embodiment, the largely double stranded nucleic acid region comprises a nucleotide sequence obtained from a small nuclear RNA (snRNA) that is U3, U2, U4 to U6, U8, U13 to U16, U18 to U21, U23 to U72, 4.5S RNAI to III, 5S RNAIII, E2 or E3. The largely double stranded nucleic acid region preferably comprises a nucleotide sequence obtained from a small nuleaolar localised RNA (snoRNA). In an embodiment of the invention, the largely double stranded nucleic acid region comprises a nucleotide sequence obtained from U6 snoRNA, most preferably from human U6 snoRNA as shown in FIG. 16.

“Small nuclear RNAs” (snRNA) are small, relatively conserved RNA molecules found in eukaryotic cells, typically between 50 and 500 nucleotides in length and usually between 50 and 200 nucleotides in length, which are predominantly or exclusively nuclear localized. They typically contain regions of largely double-stranded RNA which are important for their function. SnRNAs include the small nucleolar localized RNAs (snoRNA). They do not encode proteins (non-messenger RNAs), and are generally involved in RNA splicing, RNA modification including methylation and pseudouridylation, folding or transport processes in the cell. Many snRNA have been discovered and some are localized in subnuclear compartments such as the nucleolus. They may be complexed with proteins and assembled into ribonucleoprotein particles in the cytoplasm before import into the nucleus as in, for example, metazoan cells.

There are over 200 nucleolar-specific snRNA that include abundant molecules such as those designated U3, U8, and U13 RNAs. Extranucleolar-nuclear-specific RNAs include 4.5S RNA I, II, III, 5S RNA III, U1, U2, U4, U5, and U6, in addition to over 500 different RNA species reported up to now. Others include the RNA component of RNAse P, signal recognition particle RNA that assembles with proteins in the nucleolus, and telomerase RNA. Some snoRNA and snRNA have trimethylguanosine cap structures that are unique to eukaryotes. Many snRNA have important roles in gene expression such as transcription (U3 snoRNA), RNA processing (U3, U8, U13, U14, U22, and 7-2/MRP), methylation (U14-U16, U18, U20-U21, and U24-U63), pseudouridylation (E2, E3, U19, U23, and U64-U72), and RNA splicing (U1, U2, U4, U5, and U6 snRNA). The snRNA molecules U1, U2, U4, U5 and U6 are essential components of the spliceosome complex, the ribonucleoprotein complex that carries out splicing (intron removal) for mRNA formation. Many snRNA are genes encoding snRNA are transcribed by RNA polymerase III, although some are transcribed by RNA polymerase II. Some snRNA genes are found within the introns of other, larger genes.

More than 100 snoRNA have been identified (Vitali et al., (2003). Nucl Acids Res 31:6543-51). Based on sequence and structural motifs, 113 of these RNAs could be assigned to the C/D box or H/ACA box subclasses of snoRNA. Many of the snRNA including snoRNA are encoded by multiple genes (gene families) within eukaryotes, so each snRNA type may consist of closely related sequences. For example, nine U6 loci have been identified in the human genome of which at least five members are active genes (Domitrovich and Kunkel (2003) Nucl Acids Res 31: 2344-52). The nucleolar localization element of the U6 snRNA has been identified and includes its 3′ end (Gerbi and Lange (2002) Mol Cell Biol 13: 3123-37). Localisation of this molecule depends at least in part on binding to specific nuclear proteins.

snRNA including snoRNA can readily be identified from eukaryotic cells including plant cells (Brown et al, 2003, Trends in Plant Sci 8: 42-) or animal cells as shown, for example by Huttenhofer et al (2001, EMBO J 20:2943-53). Homologs of snRNA can be obtained from other eukaryotic species by using known members as probes or as a source of primers for amplification reactions, well known in the art. Derivatives of naturally occurring snRNA can also be readily obtained by mutations, nucleotide substitutions, insertions and deletions and the like, and are useful provided that they retain their nuclear localization signals. In particular, the positions of basepairing interactions within the largely double-stranded regions of the molecules should be preserved even if the sequences of the basepairs themselves may be altered. Nuclear localization may be readily determined by techniques such as in situ hybridization or subcellular fractionation.

The largely double stranded nucleic acid region may comprise a nucleotide sequence obtained from a viroid is a Potato Spindle Tuber Viroid, Citrus Viroid species III, Citrus Viroid species IV, Hop Latent Viroid, Australian Grapevine Viroid, Tomato Planta Macho Viroid, Coconut Tinangaja Viroid, Tomato Apical Stunt Viroid, Coconut Cadang-cadang Viroid, Citrus Exocortis Viroid, Columnea Latent Viroid, Hop Stunt Viroid or Citrus Bent Leaf Viroid. The viroid can have a nucleotide sequence of SEQ ID No 3, SEQ ID No 4, SEQ ID No 5, SEQ ID No 6, SEQ ID No 7 or SEQ ID No 8. In an embodiment of the invention, the largely double stranded nucleic acid region comprises a nucleotide sequence comprising a nucleic acid-nuclear localization signal from Potato Spindle Tuber Viroid. The nucleic acid-nuclear localization signal is preferably from Potato Spindle Tuber Viroid strain RG1. In an embodiment of the invention, the nuclear localization signal comprises the nucleotide sequence of SEQ ID No 3.

The largely double stranded nucleic acid region can comprise a viroid genome nucleotide sequence of the genome nucleotide sequence of Potato Spindle Tuber Viroid, the genome nucleotide sequence of Citrus Viroid species III, the genome nucleotide sequence of Citrus Viroid species IV, the genome nucleotide sequence of Hop Latent Viroid, the genome nucleotide sequence of Australian Grapevine Viroid, the genome nucleotide sequence of Tomato Manta Macho Viroid, the genome nucleotide sequence of Coconut Tinangaja Viroid, the genome nucleotide sequence of Tomato Apical Stunt Viroid, the genome nucleotide sequence of Coconut Cadang-cadang viroid, the genome nucleotide sequence of Citrus Exocortis Viroid, the genome nucleotide sequence of Columnea Latent Viroid, the genome nucleotide sequence of Hop Stunt Viroid or the genome nucleotide sequence of Citrus Bent Leaf Viroid. In an embodiment of the invention, the largely double stranded nucleic acid region comprises a genomic nucleotide sequence of Potato Spindle Tuber Viroid.

The largely double stranded nucleic acid region preferably comprises a RNA sequence having at least 35 repeats of the trinucleotide CUG. In an embodiment of the invention, the largely double stranded nucleic acid region comprises a RNA sequence having between 44 and 2000 repeats of the trinucleotide CUG.

In one embodiment of the invention, the largely double stranded nucleic acid region operably linked to the target gene specific region is a nuclear localization signal from a viroid of the PSTVd type (Bussière et al 1996), such as PSTVd (Potato spindle tuber viroid), capable of replicating in the nucleus of the host cell or host plant cell.

In one embodiment of the invention, the largely double stranded nucleic acid region comprises the full length sequence of PSTVd strain RG1, which can conveniently be obtained by amplification from a cDNA copy of the RNA genome of the viroid using oligonucleotide primers with the nucleotide sequence

[SEQ ID N^{o} 1]
5′-CGCAGATCTCGGAACTAAACTCGTGGTTC-3
and
[SEQ ID N^{o} 2])
5′-GCGAGATCTAGGAACCAACTGCGGTTC-3′,

such as the nucleotide sequence represented in SEQ ID No 3.

It is understood that for incorporation in a RNA molecule, an additional step is required to produce the RNA molecule from the corresponding DNA molecule. Production may be achieved by transcription, e.g. in vitro transcription using a single subunit bacteriophage RNA polymerase.

It is also clear than when RNA sequences are the to be represented in an entry in the Sequence Listing or to be essentially similar or have a certain degree of sequence identity with DNA sequences represented in the Sequence Listing, reference is made to RNA sequences corresponding to the sequences in the entries, except that thymine (T) in the DNA sequence is replaced by uracil (U) in the RNA sequence. Whether the reference is to RNA or DNA sequence will be immediately apparent by the context.

Similar largely double stranded RNA structures are also found within the genomes of other nuclear-replicating viroids of the PSTVd type (or group B according to the classification by Bussière et al. 1996) and these RNA sequences may be used to similar effect. Other nuclear-replicating viroids of the PSTVd group include Citrus viroid species III, Citrus viroid species IV, Coleus viroid, Hop latent viroid (SEQ ID No 7), Australian grapevine viroid (SEQ ID No 4), Tomato planta macho viroid (SEQ ID No 6), Coconut tinangaja viroid (SEQ ID No 5), Tomato apical stunt viroid (SEQ ID No 8), Coconut cadang-cadang viroid, Citrus exocortis viroid, Columnea latent viroid, Hop stunt viroid or Citrus bent leaf viroid. These viroids are also characterized by the absence of self-splicing activity which becomes apparent by the absence of catalytic motifs such as the hammerhead motif (Bussière et al. Nucl. Acids Res. 24, 1793-1798, 1996, herein incorporated by reference.) The longest stretch of basepairing without interruption by non-basepaired nucleotides among all the PSTVd-type of viroids is 11 base pairs in size. The mismatches are usually quite evenly distributed.

Nucleotide sequences for these viroids have been compiled in a database accessible via the worldwide web (callisto.si.usherb.ca/˜jpperra or nt.arsgrin.gov/subviral) and include the following:

Potato spindle tuber viroid (PSTVd) [PSTVd.1 (Accession numbers: J02287(gb), M16826(gb), V01465(embl); 333351(gi), 333352(gi) and 62283(gi)); PSTVd.2 (Accession numbers: M38345(gb), 333354(gi)); PSTVd.3 (Accession numbers: M36163(gb), 333356(gi)); PSTVd.4 (Accession numbers: M14814(gb), 333357(gi)); PSTVd.5 (strain: S. commersonii) (Accession numbers: M25199(gb), 333355(gi)); PSTVd.6 (strain: tomato cv. Rutgers, isolate: KF440-2) (Accession numbers: X58388(embl), 61366(gi)); PSTVd.7 (mild strain KF6-M) (Accession number: M88681(gb), 333358(gi)); PSTVd.8 (strain Burdock) (Accession numbers: M88678(gb), 333360(gi)); PSTVd.9 (strain Wisconsin (WB)) (Accession numbers: M88677(gb), 333359(gi)); PSTVd.10 (strain PSTVd-N(Naaldwijk)) (Accession numbers: X17268(embl), 60649(gi)); PSTVd.11 (mild strain variant A, WA-M isolate) (Accession numbers: X52036(embl), 61365(gi)); PSTVd.12 (mild strain, F-M isolate) (Accession numbers: X52037(embl), 61367(gi)); PSTVd.13 (intermediate-severe strain, F-IS isolate) (Accession numbers: X52039(embl), 61369(gi)); PSTVd.14(severe-lethal strain, F-SL isolate) (Accession numbers: X52038(embl), 61368(gi)); PSTVd.15 (intermediate-severe strain, F88-IS isolate) as published in Herold,T et al., Plant Mol. Biol. 19, 329-333 (1992); PSTVd.16 (variant F88 or S88) (Accession numbers: X52040(embl), 61370(gi)); PSTVd.17 (individualisolate kf 5) (Accession numbers: M93685(gb), 333353(gi)); PSTVd.18 (isolate KF5) (Accession numbers: S54933(gb), 265593(gi)); PSTVd.19 (strain S-XII, variety s27) (Accession numbers: X76845(embl), 639994(gi)); PSTVd.20 (strain S-XIII, variety s23) (Accession numbers: X76846(embl), 639993(gi)); PSTVd.21 (strain M(mild)) (Accession numbers: X76844(embl), 639992(gi)); PSTVd.22 (strain 1-818, variety 14) (Accession numbers: X76848(embl), 639991(gi)); PSTVd.23 (strain 1-818, variety 13) (Accession numbers: X76847(embl), 639990(gi)); PSTVd.24 (strain PSTVd-341) (Accession numbers: Z34272(embl), 499191(gi)); PSTVd.25 (strain QF B) (Accession numbers: U23060(gb),755586(gi)) PSTVd.26 (strain QF A) (Accession numbers: U23059(gb), 755585(gi)); PSTVd.27 (strain RG 1) (Accession numbers: U23058(gb), 755584(gi)); PSTVd.28 (Accession numbers: U51895(gb), 1272375(gi)); PSTVd.29(Potato spindle tuber viroid) (Accession numbers: X97387(embl), 1769438(gi)); PSTVd.30 (strain S27-VI-24) (Accession numbers: Y09382(emb), 2154945(gi)); PSTVd.31 (strain S27-VI-19) (Accession numbers: Y09383(emb), 2154944(gi)); PSTVd.32 (strain SXIII) (Accession numbers: YO8852(emb), 2154943(gi)); PSTVd.33 (strain S27-1-8) (Accession numbers: Y09381(emb), 2154942(gi)); PSTVd.34 (strain PSTV M-VI-15) (Accession numbers: Y09577(emb), 2154941(gi)); PSTVd.35 (strain PSTV M-I-40) (Accession numbers: Y09576(emb), 2154940(gi)); PSTVd.36 (strain PSTV M-I-17) (Accession numbers: Y09575(emb), 2154939(gi)); PSTVd.37 (strain PSTV M-I-10) (Accession numbers: Y09574(emb), 2154938(gi)); PSTVd.38 (variant I4-I-42) (Accession numbers: Y09889(emb), 2154937(gi)); PSTVd.39 (variant PSTVd I2-VI-27) (Accession numbers: Y09888(emb), 2154936(gi)); PSTVd.40 (variant PSTVd I2-VI-25) (Accession numbers: Y09887(emb), 2154935(gi)); PSTVd.41 (variant PSTVd I2-VI-16) (Accession numbers: Y09886(emb), 2154934(gi)); PSTVd.42 (variant PSTVd I4-I-10) (Accession numbers: Y09890(emb), 2154933(gi)); PSTVd.43 (variant PSTVd I2-I-14) (Accession numbers: Y09891(emb), 2154932(gi)); PSTVd.44 (isolate KF7) (Accession numbers: AJ007489(emb), 3367737(gi)); PSTVd.45 (Accession numbers: AF369530, 14133876(gi)];
Group III citrus viroid (CVd-III) [CVd-III.1 (Accession numbers: S76452(gb),913161(gi)); CVd-III.2 (Australia New South Wales isolate) (Accession numbers: S75465(gb) and S76454(gb), 914078(gi) and 913162(gi)); CVd-III.3 (Accession numbers: AF123879, GI:7105753); CVd-III.4 (Accession numbers: AF123878, GI:7105752) CVd-III.5 (Accession numbers: AF123877, GI:7105751); CVd-111.6 (Accession numbers: AF123876, GI:7105750); CVd-III.7 (Accession numbers: AF123875, GI:7105749); CVd-III.8 (Accession numbers: AF123874, GI:7105748); CVd-III.9 (Accession numbers: AF123873, GI:7105747); CVd-III.10 (Accession numbers: AF123872, GI:7105746); CVd-III.11 (Accession numbers: AF123871, GI:7105745); CVd-III.12 (Accession numbers: AF123870, GI:7105744); CVd-III.13 (Accession numbers: AF123869, GI:7105743); CVd-III.14 (Accession numbers: AF123868, GI:7105742); CVd-III.15 (Accession numbers: AF123867, GI:7105741); CVd-III.16 (Accession numbers: AF123866, GI:7105740); CVd-III.17 (Accession numbers: AF123865, GI:7105739); CVd-III.18 (Accession numbers: AF123864, GI:7105738) CVd-III.19 (Accession numbers: AF123863, GI:7105737); CVd-III.20 (Accession numbers: AF123860, GI:7105736); CVd-III.21 (Accession numbers: AF123859, GI:7105735); CVd-III.22 (Accession numbers: AF123858, GI:7105734); CVd-III.23 (Accession numbers: AB054619, GI:13537479); CVd-III.24 (Accession numbers: AB054620, GI:13537480); CVd-III.25 (Accession numbers: AB054621, GI:13537481); CVd-III.26 (Accession numbers: AB054622, GI:13537482); CVd-III.27 (Accession numbers: AB054623, GI:13537483); CVd-III.28 (Accession numbers: AB054624, GI:13537484); CVd-III.29 (Accession numbers: AB054625, GI:13537485); CVd-III.30 (Accession numbers: AB054626, GI:13537486); CVd-III.31 (Accession numbers: AB054627, GI:13537487); CVd-III.32 (Accession numbers: AB054628, GI:13537488); CVd-III.33 (Accession numbers: AB054629, GI:13537489); CVd-III.34 (Accession numbers: AB054630, GI:13537490); CVd-III.35 (Accession numbers: AB054631, GI:13537491); CVd-III.36 (Accession numbers: AB054632, GI:13537492); CVd-111.37 (Accession numbers: AF416552, GI:15811643); CVd-III.38 (Accession numbers: AF416553, GI:15811644); CVd-III.39 (Accession numbers: AF416374, GI:15788948); CVd-III.40 (Accession number: AF434680)];
Citrus viroid IV (CVdIV) [CVdIV.1 (Accession numbers: X14638(embl), 59042(gi))]
Coleus blumei-1 viroid (CbVd-1) [CbVd.1 (Coleus blumei viroid 1 (CbVd 1),strain cultivar Bienvenue, german isolate) (Accession numbers: X52960(embl), 58844(gi)); CbVd.2 (Coleus yellow viroid (CYVd), Brazilian isolate) (Accession numbers: X69293(embl), 59053(gi)); CbVd.3 (Coleus blumei viroid 1-RG stem-loop RNA.) (Accession numbers: X95291(embl), 1770104(gi)); CbVd.4 (Coleus blumei viroid 1-RL RNA) (Accession numbers: X95366(embl), 1770106(gi))]
Coleus blumei-2 viroid (CbVd-2) [CbVd.1 (Coleus blumei viroid 2-RL RNA) (Accession numbers: X95365(embl), 1770107(gi)); CbVd.2 (Coleus blumei viroid CbVd 4-1 RNA) (Accession numbers: X97202(embl), 1770109(gi))] Coleus blumei-3 viroid (CbVd-3) [CbVd.1 (Coleus blumei viroid 3-RL) (Accession mumbers: X95364(embl), 1770108(gi)); CbVd.2 (Coleus blumei viroid 8 from the Coleus blumei cultivar ‘Fairway Ruby’) (Accession numbers: X57294(emb 1), 780766(gi)); CbVd.3 (Coleus blumei viroid 3-FR stem-loop RNA, from the Coleus blumei cultivar ‘Fairway Ruby’) (Accession numbers: X95290(embl), 1770105(gi))]

### Hop Latent Viroid (HLVd)

[HLVd.1 (Accession numbers: X07397(embl), 60259(gi)); HLVd.2 (‘thermomutant’ T15) (Accession numbers: AJ290404(gb), 13872743(gi)); HLVd.3 (‘thermomutant’T40) (Accession numbers: AJ290405.1(gb), 13872744(gi)); HLVd.4 (‘thermomutant’ T50) (Accession numbers: AJ290406(gb), 13872745(gi)); HLVd.5 (‘thermomutant’ T59) (Accession numbers: AJ290406(gb), 13872746(gi)); HLVd.6 (‘thermomutant’ T61) (Accession numbers: AJ290408(gb) 13872747(gi)); HLVd.7 (‘thermomutant’ T75) (Accession numbers: AJ290409(gb), 13872748(gi)); HLVd.8 (‘thermomutant’ T92) (Accession numbers: AJ290410(gb), 13872749(gi)); HLVd.9 (‘thermomutant’ T218) (Accession numbers: AJ290411(gb), 13872750(gi)); HLVd.10 (‘thermomutant’ T229) (Accession numbers: AJ290412(gb), 13872751(gi))]
Australian grapevine viroid (AGVd) [AGVd.1 (Accession numbers: X17101(embl), 58574(gi))]
Tomato planta macho viroid (TPMVd) [TPMVd.1 (Accession numbers: K00817(gb))]
Coconut tinangaja viroid (CTiVd) [CTiVd.1 (Accession numbers: M20731(gb), 323414(gi))]
Tomato apical stunt viroid (TASVd) [TASVd.1 (Accession numbers K00818(gb), 335155(gi)); TASVd.2 (strain: indonesian) (Accession numbers: X06390(embl), 60650(gi)); TASVd.3(Tomato apical stunt viroid-S stem-loop RNA.) (Accession numbers: X95293(embl), 1771788(gi))]
Cadang-cadang coconut viroid (CCCVd) [CCCVd.1 (isolate baao 54, ccRNA 1 fast) (Accession numbers: J02049(gb), 323275(gi)); CCCVd.2 (isolate baao 54, ccRNA 1 fast) (Accession numbers: J02050(gb), 323276(gi)); CCCVd.3 (isolate baao 54, ccRNA 1 slow) (Accession numbers: J02051(gb), 323277(gi)); CCCVd.4 (isolates Ligao 14B, 620C, 191D and T1, ccRNA 1 fast) (Haseloff et al. Nature 299, 316-321 (1982)) CCCVd.5 (isolates Ligao T1, ccRNA 1 slow) (Haseloff et al. Nature 299, 316-321 (1982)); CCCVd.6 (isolates Ligao 14B, ccRNA 1 slow) (Haseloff et al. Nature 299, 316-321 (1982)); CCCVd.7 (isolate San Nasciso, ccRNA 1 slow) (Haseloff et al. Nature 299, 316-321 (1982))]
Citrus exocortis viroid (CEVd) [CEVd.1 (cev from gynura) (Accession numbers: J02053(gb), 323302(gi)); CEVd.2 (strain A) (Accession numbers: M34917(gb), 323305(gi)); CEVd.3 (strain de25) (Accession numbers: K00964(gb), 323303(gi)); CEVd.4 (strain de26) (Accession numbers: K00965(gb), 323304(gi)); CEVd.5 (CEV-JB) (Accession numbers: M30870(gb), 484119(gi)); CEVd.6 (CEV-JA) (Accession numbers: M30869(gb), 484118(gi)); CEVd.7 (Accession numbers: M30871(gb), 484117(gi)); CEVd.8 (CEV-A) (Accession numbers: M30868(gb), 484116(gi)); CEVd.9 (Visvader, J. E. and Symons, R. H. Nucleic Acids Res. 13, 2907-2920 (1985)) CEVd.10 (Visvader, J. E. and Symons, R. H. Nucleic Acids Res. 13, 2907-2920 (1985)); CEVd.11 (Visvader, J. E. and Symons, R. H. Nucleic Acids Res. 13, 2907-2920 (1985)); CEVd.12 (Visvader, J. E. and Symons, R. H. Nucleic Acids Res. 13, 2907-2920 (1985)); CEVd.13 (Visvader, J. E. and Symons, R. H. Nucleic Acids Res. 13, 2907-2920 (1985)); CEVd.14 (Visvader, J. E. and Symons, R. H. Nucleic Acids Res. 13, 2907-2920 (1985)); CEVd.15 (Visvader, J. E. and Symons, R. H. Nucleic Acids Res. 13, 2907-2920 (1985)); CEVd.16 (Visvader, J. E. and Symons, R. H. Nucleic Acids Res. 13, 2907-2920 (1985)); CEVd.17 (Visvader, J. E. and Symons, R. H. Nucleic Acids Res. 13, 2907-2920 (1985)); CEVd.18 (Visvader, J. E. and Symons, R. H. Nucleic Acids Res. 13, 2907-2920 (1985)); CEVd.19 (Visvader, J. E. and Symons, R. H. Nucleic Acids Res. 13, 2907-2920 (1985)); CEVd.20 (Visvader, J. E. and Symons, R. H. Nucleic Acids Res. 13, 2907-2920 (1985)); CEVd.21 (cev-j classe B) (Visvader, J. E. and Symons, R. H. Nucleic Acids Res. 13, 2907-2920 (1985)); CEVd.22 (Grapevine viroid (GV)) (Accession numbers: Y00328(embl), 60645(gi)); CEVd.23 (CEVd-t) (Accession numbers: X53716(embl), 433503(gi)); CEVd.24 (CEVcls, isolate tomato hybrid callus) (Accession numbers: S67446(gb), 141247(gi)); CEVd.25 (CEV D-92) (Accession numbers: S67442(gb), 141248(gi)); CEVd.26 (CEVt, isolate tomato hybrid) (Accession numbers: S67441(gb), 141246(gi)); CEVd.27 (CEVt, isolate tomato) (Accession numbers: S67440(gb), 141245(gi)); CEVd.28 (CEVg, isolate Gynura) (Accession numbers: S67438(gb), 141244(gi)); CEVd.29 (CEVc, isolate citron) (Accession numbers: S67437(gb), 141243(gi)); CEVd.30 (strain CEVd-225) (Accession numbers: U21126(gb), 710360(gi)); CEVd.31 (isolate broad bean, Vicia faba L.) (Accession numbers: S79831(gb),1181910(gi)); CEVd.32 (variant obtain after inoculation tomato with cevd.31) (Fagoaga et al. J. Gen. Virol. 76, 2271-2277 (1995)); CEVd.33 (Fagoaga et al. J Gen. Virol. 76, 2271-2277 (1995)); CEVd.34 (Accession numbers: AF298177, 15419885(gi)); CEVd.35 (Accession numbers: AF298178, 15419886(gi)); CEVd36 (Accession: AF428058) (Citrus exocortis viroid isolate 205-E-1 Uy, complete genome.); CEVd.37 (Accession: AF428059) (Citrus exocortis viroid isolate 205-E-2 Uy, complete genome.); CEVd.38 (Accession: AF428060) (Citrus exocortis viroid isolate 205-E-5 Uy, complete genome.); CEVd.39 (Accession: AF428061) (Citrus exocortis viroid isolate 205-E-7 Uy, complete genome.); CEVd.40 (Accession: AF428062) (Citrus exocortis viroid isolate 54-E-1 Uy, complete genome.); CEVd.41 (Accession: AF428063) (Citrus exocortis viroid isolate 54-E-3 Uy, complete genome.); CEVd.42 (Accession: AF428064) (Citrus exocortis viroid isolate 54-E-18 Uy, complete genome.); CEVd.43 (Accession: AF434678) (Citrus exocortis viroid, complete genome.)]
Columnea latent viroid (CLVd) [CLVd.1 (Accession numbers: X15663(embl), 58886(gi)); CLVd.2 (CLVd-N, individual isolate Nematanthus) (Accession numbers: M93686(gb), 323335(gi)); CLVd.3 (Columnea latent viroid-B stem-loop RNA) (Accession numbers: X95292(embl), 1770174(gi))]
Citrus bent leaf viroid (CBLVd) [CBLVd.1 (CVd-Ib) (Accession numbers: M74065(gb), 323413(gi)); CBLVd.2 (strain CBLVd-225) (Accession numbers: U21125(gb), 710359(gi)); CBLVd.3 (viroid Ia genomic RNA, isolate: Jp) (Accession numbers: AB006734(dbj), 2815403(gi)); CBLVd.4 (viroid Ib genomic RNA, isolate: P2) (Accession numbers: AB006735(dbj), 2815401(gi)); CBLVd.5 (viroid Ia genomic RNA) (Accession numbers: AB006736(dbj), 2815402(gi)); CBLVd.6 (Citrus Viroid Ia clone 17) (Accession numbers: AF040721(gb), 3273626(gi)); CBLVd.7 (Citrus Viroid Ia clone 18) (Accession numbers: AF040722(gb), 3273627(gi)); CBLVd.8 (Citrus bent leaf viroid isolate 201-1-1 Uy, complete genome.) (Accession: AF428052); CBLVd.9 (Citrus bent leaf viroid isolate 201-1-2 Uy, complete genome.) (Accession: AF428053); CBLVd.10 (Citrus bent leaf viroid isolate 201-1-Uy, complete genome.) (Accession: AF428054); CBLVd.11 (Citrus bent leaf viroid isolate 205-1-1 Uy, complete genome.) (Accession: AF428055); CBLVd.12 (Citrus bent leaf viroid isolate 205-1-3 Uy, complete genome.) (Accession: AF428056); CBLVd.13 (Citrus bent leaf viroid isolate 205-1-4 Uy, complete genome.) (Accession: AF428057)]
Hop stunt viroid (HSVd) [HSVd.h1 (Japanese type strain) (Accession numbers: X00009(embl), 60684(gi)); HSVd.h2 (Japanese strain, variant 2) (Lee et al. Nucleic Acids Res. 16, 8708-8708 (1988)); HSVd.h3 (Korean strain) (Accession numbers: X12537(embl),60421(gi)); HSVd.gl (Grapevine viroid (GVVd), isolate SHV-g(GV)) (Accession numbers: M35717(gb), 325405(gi)); HSVd.g2 (strain: German cultivar Riesling) (Accession numbers: X06873(embl), 60422(gi)); HSVd.g3 (strain: isolated from Vitis vinifera Rootstock 5BB) (Accession numbers: X15330(embl), 60648(gi)); HSVd.g4 (isolate grapevine (HSVdg), variant Ia) (Accession numbers: X87924(embl), 897764(gi)); HSVd.g5 (isolate grapevine (HSVdg), variant Ib) (Accession numbers: X87923(embl), 897765(gi)); HSVd.g6 (isolate grapevine (HSVdg), variant Ic) (Accession numbers: X87925(embl), 897766(gi)); HSVd.g7 (isolate grapevine (HSVdg), variant Id) (Accession numbers: X87926(embl), 897767(gi)); HSVd.g8 (isolate grapevine (HSVdg), variant Ie) (Accession numbers: X87927(embl), 897768(gi)); HSVd.g9 (isolate grapevine (HSVdg), variant IIa) (Accession numbers: X87928(embl), 897769(gi)); HSVd.cit1 (variant 1, isolate HSV-cit) (Accession numbers: X06718(embl), 60646(gi)); HSVd.cit2 (variant 2, isolate HSV-cit) (Accession numbers: X06719(embl), 60647(gi)); HSVd.cit3 (HSV.citrus) (Accession numbers: X13838(embl), 60418(gi)); HSVd.cit4(Accession numbers: U02527(gb), 409021(gi)); HSVd.cit5 (Hsu et al. Virus Genes 9, 193-195 (1995)); HSVd.cit6 city (Hsu et al. Virus Genes 9, 193-195 (1995)); HSVd.cit7 (isolate CVd-IIa or E819) (Accession numbers: AF131248(gb)); HSVd.cit8 (isolate CVd-IIb or Ca902) (Accession numbers: AF131249(gb)); HSVd.cit9 (isolate CVd-IIc or Ca905) (Accession numbers: AF131250(gb)); HSVd.cit10 (isolate Ca903) (Accession numbers: AF131251(gb)); HSVd.cit11 (isolate CA909) (Accession numbers: AF131252(gb)); HSVd.cit12 (cachexia isolate X-701-M) (Accession numbers: AF213483(gb), 12082502(gi)); HSVd.cit13 (cachexia isolate X-701-1) (Accession numbers: AF213484(gb), 12082503(gi)); HSVd.cit14 (cachexia isolate X-701-2) (Accession numbers: AF213485(gb) 12082504(gi)); HSVd.cit15 (cachexia isolate X-701-3) (Accession numbers: AF213486(gb), 12082505(gi)); HSVd.cit16 (cachexia isolate X-704-M) (Accession numbers: AF213487(gb), 12082506(gi)); HSVd.cit17 (cachexia isolate X-704-1) (Accession numbers: AF213488(gb), 12082507(gi)); HWd.cit18 (cachexia isolate X-704-2) (Accession numbers: AF213489(gb), 12082508(gi)); HSVd.cit19 (cachexia isolate X-704-3) (Accession numbers: AP213490(gb), 12082509(gi)); HSVd.cit20 (cachexia isolate X-707-M) (Accession numbers: AF213491(gb), 12082510(gi)); HSVd.cit21 (cachexia isolate X-707-1) (Accession numbers: AF213492(gb), 12082511(gi)); HSVd.cit22 (cachexia isolate X-707-2) (Accession numbers: AF213493(gb), 12082512(gi)); HSVd.cit23 (cachexia isolate X-707-3) (Accession numbers: AF213494(gb), 12082513(gi)); HSVd.cit24 (cachexia isolate X-707-4) (Accession numbers: AF213495(gb), 12082514(gi)); HSVd.cit25 (cachexia isolate X-712-M) (Accession numbers: AF213496(gb), 12082515(gi)); HSVd.cit26 (cachexia isolate X-712-1) (Accession numbers: AF213497(gb), 12082516(gi)); HSVd.cit27 (cachexia isolate X-712-2) (Accession numbers: AF213498(gb), 12082517(gi)); HSVd.cit28 (cachexia isolate X-712-3) (Accession numbers: AF213499(gb), 12082518(gi)); HSVd.cit29 (cachexia isolate X-715-M) (Accession numbers: AF213500(gb), 12082519(gi)); HSVd.cit30 (cachexia isolate X-715-1) (Accession numbers: AF213501(gb), 12082520(gi)); HSVd.cit31 (cachexia isolate X-715-2) (Accession numbers: AF213502(gb), 12082521(gi)); HSVd.cit32 (CVd-Iia (117)) (Accession numbers: AF213503(gb), 12082522(gi)); HSVd.cit33 (isolate CVd-IIa 17uy) (Accession numbers: AF359276(gb), 13991644(gi)); HSVd.cit34 (isolate CVd-IIa 11uy) (Accession numbers: AF359275(gb), 13991643(gi)); HSVd.cit35 (isolate CVd-IIa 10uy) (Accession numbers: AF359274(gb), 13991642(gi)); HSVd.cit36 (isolate CVd-Ib 10uy) (Accession numbers: AF359273(gb), 13991641(gi)); HSVd.cit37 (isolate CVd-Ib 5uy) (Accession numbers: AF359272(gb), 13991640(gi)); HSVd.cit38 (isolate CVd-Ib 3uy) (Accession numbers: AF359271(gb), 13991639(gi)); HSVd.cit39 (isolate CVd-Ib 2uy) (Accession numbers: AF359270(gb), 13991638(gi)); HSVd.cit40 (isolate CVd-IIa) (Accession numbers: X69519(embl), 2369773(gi)); HSVd.cit41 (isolate CVd-TIb) (Accession numbers: X69518(embl), 2369774(gi)); HSVd.cit42 (isolate CVd-IIa 54-2-1) (Accession numbers: AF416554, 15811645(gi)); HSVd.cit43 (isolate CVd-IIa 54-2-2) (Accession numbers: AF416555, 15811646(gi)); HSVd.cit44 (isolate CVd-IIa 205-2-4) (Accession numbers: AF416556, 15811647(g0); HSVd.cit45 (isolate CVd-IIa 205-2-1) (Accession numbers: AF416557, 15811648(gi)); HSVd.p1 (HSV-peach (A9)) (Accession numbers: D13765(dbj), 221254(gi)); HSVd.p2 (HSV-plum and HSV-peach (AF) isolate) (Accession numbers: D13764(dbj), 221255(gi)); HSVd.p3 (cv. Jeronimo J-16 from Spain) (Accession numbers: Y09352(embl),1684696(gi)); HSVd.apr1 (cv. Rouge de Roussillon from France) (Accession numbers: Y08438(embl), 2462494(gi)); HSVd.apr2 (unknown cultivar from Spain) (Accession numbers: Y08437 (embl), 2462495(gi)); HSVd.apr3 (cv. Bulida from Spain) (Accession numbers: Y09345(embl),1684690(gi)); HSVd.apr4 (cv. Bulida from Spain) (Accession numbers: Y09346(embl),1684691(gi)); HSVd.apr5 (cv. Bulida d'Arques from Spain) (Accession numbers: Y09344(embl),1684692(gi)); HSVd.apr6 (cv. Pepito del Rubio from Spain) (Accession numbers:Y09347(embl), 1684697(gi)); HSVd.apr7 (cv. Pepito del Rubio from Spain) (Accession numbers: 09348(embl), 1684699(gi)); HSVd.apr8 (cv. Pepito del Rubio from Spain) (Accession numbers: Y09349(embl), 684698(gi)); HSVd.apr9 (cv. Camino from Morocco) (Accession numbers: AJ297825(gb), 10944963(gi)); HSVd.apr10 (cv. Camino from Morocco) (Accession numbers: AJ297826(gb), 10944964(gi)); HSVd.aprll (cv. Camino from Morocco) (Accession numbers: AJ297827(gb), 10944965(gi)); HSVd.apr12 (cv. Camino from Morocco) (Accession numbers: AJ297828(gb), 10944966(gi)); HSVd.apr13 (cv. Camino from Morocco) (Accession numbers: AJ297829(gb), 10944967(gi)); HSVd.apr14 (cv. Septik from Turkey) (Accession numbers: AJ297830(gb), 10944968(gi)); HSVd.apr15 (cv. Monaco bello from Cyprus) (Accession numbers: AJ297831(gb), 10944969(gi)); HSVd.apr16 (cv.Cafona from Cyprus) (Accession numbers: AJ297832(gb), 10944970(gi)); HSVd.apr17 (cv.Cafona from Cyprus) (Accession numbers: AJ297833(gb), 10944971(gi)); HSVd.apr18 (cv.Boccuccia spinosa from Cyprus) (Accession numbers: AJ297834(gb), 10944972(gi)); HSVd.apr19 (cv. Palumella from Cyprus) (Accession numbers: AJ297835(gb), 10944973(gi)); HSVd.apr20 (cv. Palumella from Cyprus) (Accession numbers: AJ297836(gb), 10944974(gi)); HSVd.apr21 (cv.Camino from Cyprus) (Accession numbers: AJ297837(gb), 10944975(gi)); HSVd.apr22 (cv.Kolioponlou from Greece) (Accession numbers: AJ297838(gb), 10944976(gi)); HSVd.apr23 (cv. Bebecou Paros from Greece) (Accession numbers: AJ297839(gb), 10944977(gi)); HSVd.apr24 (cv. Bebecou Paros from Greece) (Accession numbers: AJ297840(gb), 10944978(gi)); HSVd.c1 (Cucumber pale fruit viroid (CPFVd), isolate HSV-cucumber) (Accession numbers: X00524(embl), 60644(gi)); HSVd.c2 (Cucumber pale fruit viroid (CPFVd)) (Accession numbers: X07405(embl), 59015(gi)); HSVd.alm1 (Accession numbers: AJ011813(emb), 3738118(gi)); HSVd.alm2 (Accession numbers: AJ011814(emb), 3738119(gi)); HSVd. Citrus viroid II, complete genome (Accession number: AF434679)]. All these nucleotide sequences are herein incorporated by reference.

As will be immediately apparent from the above list, viroids are extremely prone to sequence variations, and such natural variants can also be used for the currently described methods and means, particularly if they retain the capacity to be transported to the nucleus, together with any operably linked nucleic acid sequence.

In addition to the natural variations in viroid nucleotide sequences, variants may be obtained by substitution, deletion or addition of particular nucleotides, and such variants may also be suitable for the currently described methods and means, particularly if they retain the capacity to be transported to the nucleus, together with any operably linked nucleic acid sequence.

Further, smaller RNA regions derived from the viroid nucleotide sequences, and variants thereof can be used for the current invention which are capable of being transported to the nucleus together with any operably linked nucleic acid sequence. The RNA region obtained from the PSTVd type viroid may comprise at least 100 nucleotides, preferably at least 150, 200, 250 or 300 nucleotides obtained from the viroid. In an embodiment of the invention, the RNA region comprises 95-100% of the full length sequence of the PSTVd type viroid.

The capacity of both smaller regions and variants derived from viroid nucleotide sequences to be transported to the nucleus of a host cell, such as a plant cell, can be determined using the assay described by Zhou et al. 2001, J. Gen Virology, 82, 1491-1497. Briefly, the assay comprises introducing a marker coding region, such as GFP, comprising an intervening sequence in the coding region of the marker gene, into the host cell by means of a viral RNA vector that replicates in the cytoplasm of the host cell. When a functional nuclear localization signal is introduced (conveniently inserted in the intervening sequence), the viral RNA vector comprising the marker gene is imported into the nucleus, where the intron can be removed and the spliced RNA returned to the cytoplasm. The spliced RNA can be detected by the translation into GFP protein, as well as by RNA analysis methods (e.g. RT-PCR) to confirm the absence of the intron from the spliced RNA molecules.

Furthermore, the hepatitis delta virus (HDV) RNA is a single stranded circular RNA of about 1679 nucleotides which is very similar to the viroids of the PSTVd-type in that is localized in the nucleus, forming essentially unbranched rod-like structures (Kuo et al., J. Virol 62:1855-1861 (1988)), and may also be used according to the invention. The nucleotide sequence of human hepatitis delta virus RNA is disclosed in U.S. Pat. No. 5,932,219, which is herein incorporated by reference. The HDV RNA, which is of a negative sense polarity (the antigenomic strand is the sense strand), is replicated by a rolling circle mechanism to create the antigenome, which is also essentially unbranched rod-like, and is transcribed to form a subantigenomic message that encodes the small delta antigen (HDAg-S). The subantigenomic message RNA lacks the characteristic unbranched rod-like structure of the genome or antigenomic RNAs (Hsieh et al., J. Virol 64:3192-3198 (1990)). Other, related RNAs which form essentially unbranched rod-like structures can also be used in the invention, including molecules with deletions or substitutions, so long as the largely double stranded character of the structure is maintained by substantial but not complete base pairing.

In another embodiment of the invention, the largely double stranded nucleic acid region comprises a trinucleotide repeat region comprising CUG, CAG, GAC or GUC trinucleotide repeats. As used herein “trinucleotide repeat region” is the portion of a nucleic acid molecule comprising a number of CUG, CAG, GAC or GUC trinucleotides. In an embodiment, the trinucleotides are repeated without intervening sequences, although short regions of 1 to 20-30 nucleotides not consisting of CUG, CAG, GAC or GUC trinucleotides may be present occasionally between the trinucleotides. In an embodiment, the trinucleotide repeat region comprises at least 35 CUG, CAG, GAC or GUC trinucleotides, or at least 44 such trinucleotides, or any number between 50 and 2000 trinucleotides. Conveniently the copy number of the trinucleotides should not exceed 100 or 150. In an embodiment, the trinucleotide repeat region comprises not more than 20, or 10, or 6 nucleotides other than the trinucleotides, in a region of at least 105 nucleotides. It is preferred that the trinucleotide is CUG.

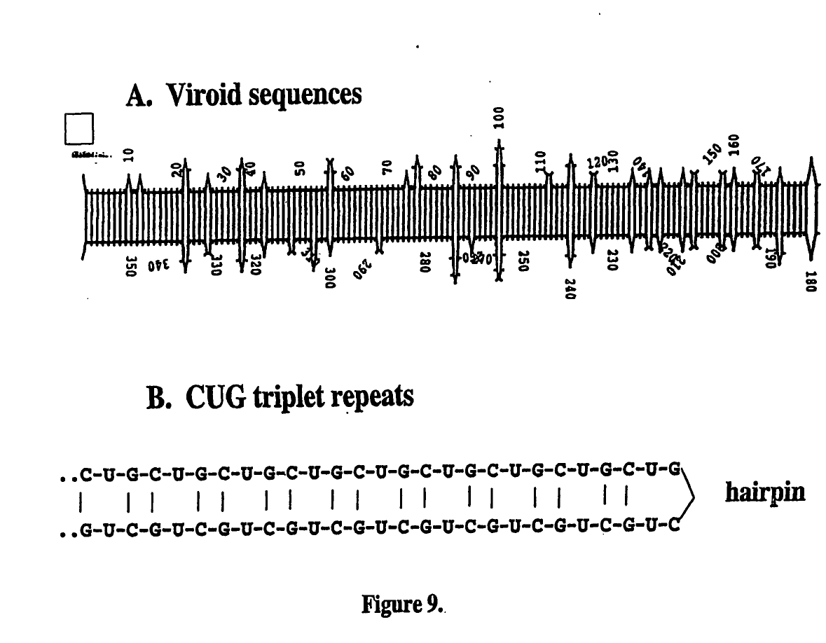

Without intending to limit the invention to a particular mode of action, it is taught that such trinucleotide repeats form rod-like structures by imperfect base-pairing (for example, FIG. 9) which function as nuclear retention signal, possibly by sterically blocking RNA export through nuclear pores, as well as not activating double stranded RNA dependent protein kinase PKR (Davis et al, 1997 Proc. Natl. Acad. Sci. 94: 7388-7393; Tian et al. 2000 RNA 6: 79-87; Koch and Lefert 1998 J. Theor. Biol. 192: 505-514). Furthermore, the rod-like structures formed by the trinucleotide repeats, and those formed by the other largely double stranded nucleic acid regions disclosed herein, may not activate and/or be substrates for adenosine deaminases that act on RNA in the nucleus, such as ADAR1-S (small form of adenosine deaminase that acts on RNA) or ADAR2. ADARs are enzymes that act on dsRNA and convert adenosines to inosines, and a 15 basepair double stranded region with not more than one mismatch is sufficient as substrate in vertebrate cells (Herbert and Rich, Proc Natl Acad Sci USA 98:12132-12137, (2001) herein incorporated by reference). Some ADARs are induced by interferons.

With regard to trinucleotide repeats and human disease, it is of interest to note that some mutations associated with human disease involve trinucleotide repeat expansions, in particular in the Huntington Disease (HD) gene and the ataxin 3 gene which are both associated with the development of neurodegenerative diseases. The mutant genes include expanded repeat regions with more than 35 repeat copies, with CUG or CAG predominant. For the HD gene encoding huntingtin, fewer than 29 triplet repeats are within the normal 5 range, 29-35 triplets are considered intermediate length, while alleles with more than 36 triplets are considered to be mutant expansions. There is a striking correlation between the length of the triplet repeat expansion and the age of onset of disease. Even though the major pathogenic mechanism of HD is thought to involve a toxic gain-of-function by the mutant protein containing polyglutamine tracts, we suggest here that the expanded repeat region in the mutant RNA may cause excessive nuclear localization of the RNA which may also be associated with the molecular basis of the disease or of other diseases such as fragile X syndrome, where triplet repeat expansion is thought to lead to impaired transcription of the FMRI gene (Kaufmann and Reiss, Am J Med Genet 88:11-24, 1999).

The methods of the present invention preferably, further comprises the step of identifying a cell of an animal, fungus or protist, wherein the expression of the target gene is down regulated. The method may further comprise a step of identifying or selecting or isolating a cell, preferably an animal cell, or its progeny wherein the expression of the target gene is down regulated in the cell. The step of identifying, selecting or isolating such cells may be on the basis of the reduced gene expression, on the presence of the provided nucleic acid molecule in the cell, or on a phenotype conferred on the cell by the presence of the nucleic acid molecule.

In yet another aspect of the invention there is provided a method of identifying or characterising a nucleic acid-nuclear localization signal in an isolated nucleic acid molecule, comprising the steps of

(a) providing a first a cell with a first chimeric nucleic acid molecule wherein the molecule comprises
(i) a target-gene specific region comprising a nucleotide sequence of at least about 16 consecutive nucleotides having at least about 94% sequence identity with the complement of 16 consecutive nucleotides from the nucleotide sequence of transcribed nucleic acid sequence of the target gene,
wherein the target gene is a reporter gene, a pathogenic animal virus gene, a cancer-related gene, an oncogene, an immunomodulatory gene, a gene encoding a cytokine, growth factor, enzyme or a transcription factor, and
(ii) a largely double stranded nucleic acid region comprising a nucleotide sequence obtained from the isolated nucleic acid molecule; and
(b) providing a second cell with a second nucleic add molecule, comprising the antisense region but not the largely double stranded nucleic acid region; and
(c) determining the extent of down-regulation of the target gene expression in the first cells in the presence of the first chimeric nucleic acid molecule and the second cells in the presence of the second nucleic add molecule, wherein the first cell and the second cell is of an animal, fungus or protist.

In a further embodiment, the method may be used to compare the efficiency of nuclear localization or retention of nucleic acids comprising putative nuclear localization signals obtained from a variety of sources. In a further embodiment, the target gene encodes a reporter molecule or protein. The reporter may be firefly luciferase, Renilla luciferase, β-galactosidase, β-glucuronidase, chloramphenicol acetyltransferase (CAT), alkaline phosphatase or human growth hormone.

In an embodiment of the invention, the nuclear localization signal is a property of a nucleotide sequence obtained from a viroid, satellite RNA such as hepatitis delta virus RNA, or a nucleotide sequence comprising at least 35 repeats of a trinucleotide which is CUG, CAG, GAC or GUC. The viroid is preferably of the PSTVd type.

CUG repeats may be particularly suited to increase the efficiency of antisense-mediated gene silencing when the chimeric nucleic acid molecules comprising such CUG repeats can be delivered to the nucleus of the host cell e.g. Through transcription of a chimeric gene encoding such RNA, as hereinafter described.

Although the largely double stranded RNA region such as the PSTVd-type viroid derived nuclear location signals or the trinucleotide repeats can conveniently be located at the 3′ end of the target specific chimeric nucleic acid molecule, it is expected that the location of the largely double stranded nucleic acid region is of little importance. Hence, largely double stranded nucleic acid regions may also be located at the 5′ end of the chimeric nucleic acid molecule preferably at the 3′ end or even in the middle of such a molecule.

It was also unexpectedly found that the efficiency of antisense-mediated down regulation of gene expression, wherein an antisense nucleic acid was operably linked to a largely double stranded nucleic acid region, could be further enhanced by inclusion of an intron sequence in the chimeric nucleic acid molecule provided to the host cell. Again, the location of the intron in the chimeric nucleic acid molecule with respect to both the target gene specific region as well as the largely double-stranded nucleic acid region is expected to have little effect on the efficiency. In fact, it is expected that the largely double stranded nucleic acid region may be located within the intron sequence.

As used herein, an “intron” or intervening sequence is used to refer to a region within a larger transcribed DNA region, which is transcribed in the nucleus to yield a RNA region which is part of a larger RNA, however, the RNA region corresponding to intron sequence is removed from the larger RNA when transferred to the cytoplasm. The corresponding RNA is also referred to as an intron or intervening sequence. Intron sequences are flanked by splicing sites, and synthetic introns may be made by joining appropriate splice sites to basically any sequence, having an appropriate branching point. Introns or intervening sequences which are located in 5′UTR, coding region or 3′UTR may be used.

Intervening sequences or introns should preferably be capable of being spliced in the cells, although the presence of intervening sequences which can no longer be spliced, e.g. because their guide sequences have been altered or mutated, may even further increase the efficiency of the chimeric nucleic acid molecules to down regulate the expression of a target gene. Examples of mamalian virus introns include the inton from SV40. Examples of fungal introns include the intron from the triose phosphate isomerase gene from Aspergillus.

The chimeric nucleic acid molecules of the invention and as used in the methods of the invention may comprise ribozyme domains, in particular self-cleaving ribozyme domains. For example, see Shinagawa and Ishii, Genes & Devel 17:1340-1345 (2003) who included a ribozyme domain in a dsRNA molecule. The ribozyme domain(s) may be positioned within a 5′ UTR or 3′UTR, for example to be positioned between a transcription initiation nucleotide and the target gene specific region or the largely double stranded nucleic acid region, and/or between the target gene specific region or the largely double stranded nucleic acid region and a polyadenylation signal. In the former case, cleavage activity of the ribozyme domain would remove the 5′ portion of the nucleic acid molecule including any 5′ cap structure such as a methylated guanosine nucleotide that may be added in processing of the molecule; in the latter case, cleavage would remove the polyA tail that may be added. Removal of such signals may enhance nuclear localization of the remainder of the molecule including the target gene specific region and gene silencing. The ribozyme domain may be any self-cleaving domain, preferably a hammerhead or hairpin domain, well known in the art.

It was also unexpectedly found that further provision of a chimeric sense nucleic acid molecule comprising a target-gene specific region corresponding to a portion of the transcript of the target gene further increased the efficiency of the down regulation of the expression of the target gene. The same efficiency of down regulation of the expression of a target gene could be observed if the chimeric sense nucleic acid molecule was provided with a largely double stranded nucleic acid region as herein described. The sense nucleic acid molecule may be provided to the cell together with a chimeric antisense nucleic acid molecule capable of forming a double stranded region by basepairing with the sense nucleic acid molecule.

In an another aspect of the invention there is provided a method for down regulating the expression of a target gene in a cell of an animal, fungus or protest comprising, the method comprising the step of providing the cell with a first and a second chimeric nucleic acid molecule, wherein the first chimeric nucleic acid molecule comprises an antisense target-gene specific nucleic acid region comprising a nucleotide sequence of at least about 19 consecutive nucleotides having at least about 94% sequence identity with the complement of 19 consecutive nucleotides from transcribed nucleotide sequence of the target gene; and the second chimeric nucleic acid molecule comprises a sense target-gene specific nucleic acid region comprising a nucleotide sequence of at least about 19 consecutive nucleotides having at least about 94% sequence identity to the complement of the first chimeric nucleic acid molecule; and the first and second chimeric nucleic acid molecules are capable of basepairing at least between the 19 consecutive nucleotides of the first chimeric nucleic acid molecule and the 19 consecutive nucleotides of the second chimeric nucleic acid molecule; and either the first or the second chimeric nucleic acid molecule comprises a largely double stranded nucleic acid region operably linked to the antisense target-specific nucleic acid region or to the sense target-specific nucleic acid region.

As used herein the term “antisense nucleic acid” refers to nucleic acid molecules which comprise a nucleotide sequence that is largely complementary to part of the nucleotide sequence of a biologically active RNA, usually but not exclusively mRNA, which is transcribed from the target gene. The orientation of the nucleotide sequence of the antisense nucleic acid is therefore opposite to the direction of transcription of the target gene, as is well understood in the art. Being complementary to at least part of the target gene RNA implies that the antisense nucleic acid portion is capable of basepairing to the part of the target gene RNA, preferably under physiologically relevant conditions as is well understood in the art. For example, the basepairing or “hybridisation” can occur under conditions of ionic strength and temperature normally found in cells.

“Stringent hybridisation conditions” as used herein means that hybridisation will typically occur if there is at least 90% and preferably at least 95% sequence identity between the probe and at least part of the target sequence. Examples of stringent hybridisation conditions are overnight incubation at 42° C. in a solution comprising 50% formamide, 5×SSC (1×SSC=150 mM NaCl, 15 mM trisodium citrate), 50 mM sodium phosphate (pH 7.6), 5×Denhardt's solution, 10% dextran sulfate, and 20 μg/ml denatured sheared carrier DNA such as salmon sperm DNA, followed by washing the hybridization support in 0.1×SSC/0.1% SDS at approximately 65° C. Other hybridisation and wash conditions are well known and are exemplified in Sambrook et al, Molecular Cloning: A Laboratory Manual, Second Edition, Cold Spring Harbor, N.Y. (1989), particularly chapter 11.

As used herein, “sense nucleic acid” refers to nucleic acid molecules which comprise a nucleotide sequence that is largely identical to part of the nucleotide sequence of a biologically active RNA, usually but not exclusively mRNA, which is transcribed from the target gene. That is, the orientation of the nucleotide sequence of the sense nucleic acid is the same as the direction of transcription of the transcribed RNA of the target gene.

Preferably, the first and the second chimeric nucleic acid molecules both comprise a largely double stranded nucleic acid region. The first and the second chimeric nucleic acid molecules can comprise the same largely double stranded nucleic acid region. The first and second chimeric nucleic acid molecules both preferably comprise multiple antisense or sense target-gene specific regions. The first and second chimeric nucleic acid molecules are preferably RNA molecules which are transcribed from a first and second chimeric gene.

The method may further comprise the step of identifying a cell, wherein the expression of the target gene is down regulated.

In another embodiment, both the first and second chimeric nucleic acid molecules comprise a largely double stranded nucleic acid region. Specific embodiments for the largely double stranded nucleic acid region and target-gene specific antisense nucleic acid sequence are as described elsewhere in this application. Specific embodiments for the sense nucleic acid region are similar to the specific embodiments for the antisense nucleic acid region.

In further embodiments, both the sense and antisense target gene specific regions are part of the one molecule and basepair to form a double stranded RNA structure, which is operably joined to the largely double stranded nucleic acid region. The sense and antisense target gene specific regions may be separated by a spacer region, preferably comprising nucleotides and more preferably consisting of ribonucleotides, which may comprise at least 4 nucleotides and preferably 4-20 nucleotides. In an embodiment of the invention, the spacer region comprises an intron. The intron may be spliced out of the molecule or indeed may not be spliced out if the molecule remains in the nucleus.

In a further aspect of the invention there is provided a chimeric sense nucleic acid molecule for down regulating expression of a target gene in a cell of an animal, fungus or protist in cooperation with a chimeric antisense nucleic acid molecule, the chimeric sense nucleic acid molecule comprising

(a) a sense target-gene specific nucleic acid region comprising a nucleotide sequence of at least about 19 consecutive nucleotides having at least about 94% sequence identity to a transcribble nucleotide sequence of the target gene; and
(b) a largely double stranded nucleic acid region.

The chimeric sense nucleic acid molecule may comprise a largely double stranded nucleic acid region comprising a nucleotide sequence obtained from a viroid of the Potato Spindle Tuber Viroid (PSTVd)-type, a nucleotide sequence comprising at least 35 repeats of a trinucleotide wherein the trinucleotide is CUG, CAG, GAC or GUC, a nucleotide sequence obtained from hepatitis delta RNA, or a synthetic nucleotide sequence comprising a nucleic acid-nuclear localization signal. In an embodiment, the viroid has a genome nucleotide sequence of SEQ ID No 3, SEQ ID No 4, SEQ ID No 5, SEQ ID No 6, SEQ ID No 7 or SEQ ID No 8. In an embodiment of the invention, the nucleotide sequence comprises a nucleic acid-nuclear localization signal from Potato Spindle Tuber Viroid. The chimeric sense nucleic acid molecule may comprise a viroid genome nucleotide sequence.

The chimeric sense nucleic acid molecule comprises a largely double stranded nucleic region preferably comprising a RNA sequence having at least 35 repeats of the trinucleotide CUG. In an embodiment, the largely double stranded nucleic acid region comprises between 44 and 2000 repeats of the trinucleotide CUG. The chimeric sense nucleic acid molecule preferably comprises multiple target-gene specific regions. The chimeric sense nucleic acid molecule can preferably comprises both an antisense and a sense target-gene specific region. In an embodiment, the chimeric sense nucleic acid molecule comprises an intron sequence.

The invention provides a chimeric nucleic acid molecule for down regulating the expression of a target gene in a cell of an animal, fungus or protist, wherein the molecule comprises

a) a target-gene specific region comprising a nucleotide sequence of at least about 16 consecutive nucleotides having at least about 94% sequence identity with the complement of 16 consecutive nucleotides from a transcribed nucleotide sequence of the target gene, and
b) a largely double stranded nucleic acid region,
wherein the target gene is a reporter gene, a pathogenic animal virus gene, a cancer-related gene, an oncogene, an immunomodulatory gene, a gene encoding a cytokine, growth factor, enzyme or a transcription factor or an animal disease causing gene.

The chimeric nucleic acid molecule is preferably a RNA molecule. The largely double stranded nucleic acid region of the chimeric nucleic acid molecule preferably comprises a nuclear localization signal. In an embodiment of the invention, the largely double stranded nucleic acid region comprises a nucleotide sequence obtained from a viroid of the Potato Spindle Tuber Viroid (PSTVd)-type, a nucleotide sequence comprising at least 35 repeats of a trinucleotide CUG, CAG, GAC or GUC, a nucleotide sequence obtained from hepatitis delta RNA, or a synthetic nucleotide sequence comprising a nucleic acid-nuclear localization signal. The viroid can have a nucleotide sequence of SEQ ID No 3, SEQ ID No 4, SEQ ID N° 5, SEQ ID No 6, SEQ ID No 7 or SEQ ID No 8.

The chimeric nucleic acid molecule comprises a largely double stranded nucleic acid region that may comprise a viroid genome nucleotide sequence of the genome nucleotide sequence of a viroid. In an embodiment, the largely double stranded nucleic region comprises a RNA sequence having at least 35 repeats, more preferably between 44 and 2000 repeats of the trinucleotide CUG of the trinucleotide CUG. The chimeric nucleic acid molecule preferably comprises multiple target-gene specific regions. The chimeric nucleic acid molecule preferably comprises an intron sequence. In an embodiment, the intron sequence is a ubiquitin gene intron, an actin gene intron, a triose phosphate isomerase gene intron from Aspergillus or an intron from SV40. The chimeric nucleic acid is preferably a RNA molecule produced by transcription of a chimeric DNA molecule.

The chimeric nucleic acid molecules may comprise ribonucleotides, deoxyribonucleotides or a combination of these, or non-nucleotide components. In an embodiment, the chimeric nucleic acid molecule is a RNA molecule. The term “isolated” when used in relation to a nucleic acid refers to a nucleic acid sequence that is separated from at least one contaminant nucleic acid with which it is ordinarily associated in its natural state.

Conveniently, the chimeric nucleic molecules comprising a largely double stranded nucleic acid region as herein described may be provided to the cell by introduction and possible integration of a chimeric gene, transcription of which yields such chimeric nucleic acid molecules consisting of RNA.

In yet another aspect of the invention there is provided a chimeric DNA molecule for down regulating the expression of a target gene in a a cell of an animal, fungus or protist, the chimeric DNA comprising

(a) a promoter or promoter region recognizable by RNA polymerases in the cell; operably linked to
(b) a DNA region which when transcribed yields a chimeric sense nucleic acid molecule as hereinbefore described.

The invention also provides a chimeric DNA molecule for down regulating the expression of a target gene in a cell of an animal, fungus or protist, the chimeric DNA comprising

a) a promoter or promoter region recognizable by RNA polymerases in the cell; operably linked to
b) a DNA region which when transcribed yields a RNA molecule, wherein the RNA molecule comprises
(i) a target-gene specific region comprising a nucleotide sequence of at least about 16 consecutive nucleotides having at least about 94% sequence identity with the complement of 16 consecutive nucleotides from a transcribed nucleotide sequence of the target gene, and
(ii) a largely double stranded nucleic acid region,
wherein the target gene is a reporter gene, a pathogenic animal virus gene, a cancer-related gene, an oncogene, an immunomodulatory gene, a gene encoding a cytokine, growth factor, enzyme or a transcription factor or an animal disease causing gene.

The chimeric DNA molecule preferably comprises a transcription termination and/or polyadenylation signal operably linked to the DNA region which when transcribed yields the RNA molecule. In an embodiment, the promoter or promoter region of the chimeric DNA functions in an animal cell. The promoter or promoter region is preferably a promoter recognized by a prokaryotic RNA polymerase such as a bacteriophage RNA polymerase. Depending on the host organism, the promoter or promoter region may a promoter which functions in animals, or a promoter which functions in yeast including fungi or molds. The promoter may also be a promoter or promoter region recognized by a single subunit bacteriophage RNA polymerase. In an embodiment, the chimeric DNA molecule which when expressed in a cell of an animal, fungus or protist down regulates the expression of the target gene.

As used herein, the term “promoter” denotes any nucleic acid region, preferably DNA, which is recognized and bound (directly or indirectly) by a DNA-dependent RNA-polymerase during initiation of transcription. A promoter includes the transcription initiation site, and binding sites for transcription initiation factors and RNA polymerase, and can comprise various other sites (e.g., enhancers), at which gene expression regulatory proteins may bind.

The term “regulatory region”, as used herein, means any nucleic acid region that is involved in driving transcription and controlling (i.e., regulating) the timing and level of transcription of a given nucleotide sequence, such as a DNA coding for a protein or polypeptide. For example, a 5′ regulatory region (for example a “promoter region”) includes a nucleotide sequence located upstream (i.e., 5′) of a transcribed or coding sequence and which comprises the promoter and the 5′-untranslated leader sequence. A 3′ regulatory region includes a nucleotide sequence located downstream (i.e., 3′) of a coding sequence and which comprises suitable transcription termination (and/or regulation) signals, including one or more polyadenylation signals.

In one embodiment of the invention the promoter is a constitutive promoter. In another embodiment of the invention, the promoter activity is enhanced by external or internal stimuli (inducible promoter), such as but not limited to hormones, chemical compounds, mechanical impulses, abiotic or biotic stress conditions. The activity of the promoter may also regulated in a temporal or spatial manner (tissue-specific promoters; developmentally regulated promoters).

In another particular embodiment of the invention, the promoter is a fungus-expressible promoter. As used herein, the term “fungus-expressible promoter” means a nucleotide sequence, preferably DNA, which is capable of controlling (initiating) transcription of a nucleotide sequence in a fungal cell such as but not limited to the A. nidulans trpC gene promoter, or the inducible S. cerevisiae GAL4 promoter. In this context, fungi include yeasts and molds, including S. cerevisiae and Schizosaccharomyces pombe.

In yet another particular embodiment of the invention, the promoter is an animal-expressible promoter. As used herein, the term “animal-expressible promoter” means a nucleotide sequence that is capable of controlling (initiating) transcription in an animal cell. The nucleotide sequence may comprise DNA, or RNA, for example RNA sequences from a retrovirus which is reverse-transcribed into a DNA copy before being transcribed. Animal-expressible promoters include but are not limited to SV40 late and early promoters, cytomegalovirus CMV-IE promoters, RSV-LTR promoter, retrovirus LTR promoter, Pol III type promoters such as tRNA promoter, 5S rRNA promoter, U6 snRNA promoter, histone gene promoter, metallothionein promoter and the like.

Suitable transcription termination and polyadenylation regions include but are not limited to the SV40 polyadenylation signal, the HSV TK polyadenylation signal, the terminator of the Aspergillus nidulans trpC gene and the like.

The chimeric nucleic molecules useful for the invention may also be produced by in vitro transcription. To this end, the promoter of the chimeric genes according to the invention may be a promoter recognized by a bacteriophage single subunit RNA polymerase, such as the promoters recognized by bacteriophage single subunit RNA polymerase such as the RNA polymerases derived from the E. coli phages T7, T3, I, 11, W31, H, Y, A1, 122, cro, C21, C22, and C2; Pseudomonas putida phage gh-1; Salmonella typhimurium phage SP6; Serratia marcescens phage IV; Citrobacter phage ViIII; and Klebsiella phage No. 11 [Hausmann, Current Topics in Microbiology and Immunology, 75: 77-109 (1976); Korsten et al., J. Gen Virol. 43: 57-73 (1975); Dunn et al., Nature New Biology, 230: 9496 (1971); Towle et d., J. Biol. Chem. 250: 1723-1733 (1975); Butler and Chamberlin, J. Biol. Chem., 257: 5772-5778 (1982)]. Examples of such promoters are a T3 RNA polymerase specific promoter and a T7 RNA polymerase specific promoter, respectively. A T3 promoter to be used as a first promoter in the CIG can be any promoter of the T3 genes as described by McGraw et al, Nucl. Acid Res. 13: 6753-6766 (1985). Alternatively, a T3 promoter may be a T7 promoter which is modified at nucleotide positions −10, −11 and −12 in order to be recognized by T3 RNA polymerase [(Klement et al., J. Mol. Biol. 215, 21-29 (1990)]. A preferred T3 promoter is the promoter having the “consensus” sequence for a T3 promoter, as described in U.S. Pat. No. 5,037,745. A T7 promoter which may be used according to the invention, in combination with T7 RNA polymerase, comprises a promoter of one of the T7 genes as described by Dunn and Studier, J. Mol. Biol. 166: 477-535 (1983). A preferred T7 promoter is the promoter having the “consensus” sequence for a T7 promoter, as described by Dunn and Studier (supra).

The chimeric RNA molecules provided by the invention can be produced in large amounts by contacting an acceptor vector DNA with the appropriate bacteriophage single subunit RNA polymerase under conditions well known to the skilled artisan. Modified ribonucleotides can be incorporated into the chimeric RNA molecules by using the appropriate transcription conditions and RNA polymerase, as is well known in the art (for example WO95/35102, Wieczorek et al., Bioorg Medic Chem Lett. 4:987-994 (1994); Lin et al., Nucl Acids Res 22:5229-5234 (1994)). For example, mutations have been introduced into RNA polymerases to facilitate incorporation of modified nucleotides into RNA (for example Sousa and Padilla EMBO J. 14:4609-4621 (1995)) Modified nucleotides may also be incorporated into the chimeric nucleic acid molecules by chemical synthesis.

Examples of modified nucleotides are well known in the art and are described, for example, in Uhlmann and Peyman (Chemical Reviews 90:543-(1990)) or Verma and Eckstein (Ann Rev Biochem 67:99-134 (1998)), both herein incorporated by reference. The modification to the nucleotide may be to the sugar, phosphate or base. The modification may include phosphorothioate, phosphorodithioate, phosphoroamidate, alkyl-phosphates, alkyl phosphonates and the like. The sugar units may be modified, for example fluoro, amino, methoxy, 2′-O-methyl substitutions and the like. Natural nucleoside bases may be substituted with 5-hydroxymethyl uracil, 5-amino uracil or other 5-substituted pyrimidines, and the like. The sugar-phosphate backbone may be partially replaced, for example with morpholino oligomers or with polyamide nucleic acids. The molecule may have at its 3′ and/or 5′ ends nucleotides with 3′-3′ or 5′-5′ inversions or other modifications to increase nuclease stability. The molecules may be conjugated with other molecules that provide advantageous properties, for example for increased uptake or improved pharmacokinetics, for example conjugates with polylysine, polethylene glycol or intercalators, lipids or steroids, or fluorescent compounds for marking.

The chimeric nucleic acid molecules of the invention which are RNA molecules may also be conveniently produced in procaryotic cells in an efficient manner by operably linking a procaryotic promoter to a nucleotide sequence encoding the chimeric RNA molecule. The invention therefore includes such gene constructs encoding the chimeric RNA molecules, cells comprising the gene constructs, methods of producing such chimeric RNA molecules in procaryotic cells, libraries in procaryotic cells having large numbers of clones each having a different but related gene constructs encoding the chimeric RNA molecules, and kits and reagents comprising such gene constructs or components necessary to produce such gene constructs. The chimeric genes according to the invention capable of producing chimeric RNA molecules may therefore be equipped with any prokaryotic promoter suitable for expression of the chimeric RNA in a particular prokaryotic host. The prokaryotic host can be used as a source of antisense and/or sense RNA, e.g. by feeding it to an animal, such as a nematode or an insect, in which the silencing of the target gene is envisioned and monitored by reduction of the expression of a reporter gene. In this case, it will be clear that the target gene and reporter genes should be genes present in the cells of the target organism and not of the prokaryotic host organism. The antisense and sense RNA according to the invention or chimeric genes capable of yielding such antisense or sense RNA molecules, can thus be produced in one host organism, be administered to a another target organisms (e.g. Through feeding, orally administering, as a naked DNA or RNA molecule or encapsulated in a liposome, in a virus particle or attenuated virus particle, or on an inert particle etc.) and effect reduction of gene expression in the target gene or genes in another organism.

The chimeric nucleic acid molecules of the invention may be introduced in animal cells via liposomes or other transfection agents (e.g. Clonfection transfection reagent or the CalPhos Mammalian transfection kit from ClonTech). The chimeric molecules can be introduced into the cell in a number of different ways. For example, the molecules may be administered by microinjection, bombardment by particles comprising the molecules, soaking the cell or organisms in a solution of the molecules, electroporation of cell membranes in the presence of molecules, liposome mediated delivery of the molecules and transfection mediated by chemicals such as calcium phosphate, viral infection, transformation and the like. The molecules may be introduced along with components that enhance nucleic acid uptake by the cell, stabilize the annealed strands, or otherwise increase inhibition of the target gene. In the case of a whole animal, the chimeric nucleic acid molecules may be conveniently introduced by injection or perfusion into a cavity or interstitial space of an organism, or systemically via oral, topical, parenteral (including subcutaneous, intramuscular or intravenous administration), vaginal, rectal, intranasal, ophthalmic, or intraperitoneal administration. For example, see Sorensen et al., J Mol Biol 327:761-766 (2003), McCaffrey et al., Nature 418:38-39 (2002) who used hydrodynamic transfection methods. The chimeric nucleic acid molecules may also be administered via an implantable extended release device. The chimeric nucleic acid molecule may be locally administered to relevant tissues through the use of a catheter, infusion pump or stent, with or without their incorporation in biopolymers. In each of the methods of introduction described herein, the chimeric nucleic acid may be provided directly or a gene construct encoding the chimeric nucleic add may be introduced into the cell.

The invention also aims at providing the chimeric nucleic acid molecules, which may be obtained by transcription from these chimeric genes, and which are useful for the methods according to the invention. The invention utilises a cell of an animal, fungus or protist comprising the chimeric DNA molecule of the present invention or comprising the chimeric nucleic acid molecule as hereinbefore described. In an embodiment, the cell is in vitro. The cell is preferably an animal cell that is an isolated human cell an in vitro human cell, a non-human vertebrate cell, a non-human mammalian cell, fish cell, cattle cell, goat cell, pig cell, sheep cell, rodent cell, hamster cell, mouse cell, rat cell, guinea pig cell, rabbit cell, non-human primate cell, nematode cell, shellfish cell, prawn cell, crab cell, lobster cell, insect cell, fruit fly cell, Coleapteran insect cell, Dipteran insect cell, Lepidopteran insect cell or Homeopteran insect cell.

A further aspect of the invention is a cell of an animal, fungus or protist comprising a first and a second chimeric nucleic acid molecule, wherein the first chimeric nucleic acid molecule comprises an antisense target-gene specific nucleic acid region comprising a nucleotide sequence of at least about 19 consecutive nucleotides having at least about 94% sequence identity with the complement of 19 consecutive nucleotides from transcribed nucleotide sequence of the target gene; and the second chimeric nucleic acid molecule comprises a sense target-gene specific nucleic acid region comprising a nucleotide sequence of at least about 19 consecutive nucleotides having at least about 94% sequence identity to the complement of the first chimeric nucleic acid molecule; and the first and second chimeric nucleic acid molecules are capable of basepairing at least between the 19 consecutive nucleotides of the first chimeric nucleic acid molecule and the 19 consecutive nucleotides of the second chimeric nucleic acid molecule; and either the first or the second chimeric nucleic acid molecule comprises a largely double stranded nucleic acid region operably linked to the antisense target-specific nucleic acid region or to the sense target-specific nucleic acid region.

In an embodiment, the first and the second chimeric nucleic acid molecules both comprise a largely double stranded nucleic acid region. The first and the second chimeric nucleic acid molecules preferably comprise the same largely double stranded nucleic acid region; The first and second chimeric nucleic acid molecules preferably comprise multiple antisense or sense target-gene specific regions. The first and second chimeric nucleic acid molecules are most preferably RNA molecules which are transcribed from a first and second chimeric gene.

The present invention also provides a non-human cell of an animal, fungus or protist comprising the modified cells as hereinbefore described.

The invention also provides a cell comprising the chimeric nucleic acid molecules of the invention, or containing the chimeric genes capable of producing the chimeric nucleic acid molecules of the invention. In an embodiment of the invention, the chimeric genes are stably integrated in the genome of the cells of the organism. In another embodiment, the cell is a cell that is not in a human, or not in a human or animal, for example a cell in vitro or ex vivo. The methods of the invention may exclude methods of treatment of the human body, for example wherein the cell is a cell that is not in the human body, or not in a human or animal body.

The invention also provides a cell or tissues or organs and non-human organisms containing the chimeric nucleic acids, or simultaneously sense and antisense nucleic acid molecules, preferably RNA, of which one or both of the molecules comprise a largely double stranded nucleic acid region, or chimeric genes encoding such molecules.

In another embodiment, the chimeric genes of the invention may be provided on a DNA or RNA molecule capable of autonomously replicating in the cells of the organism, such as e.g. viral vectors. The chimeric gene or the chimeric nucleic acid molecule may be also be provided transiently to the cells of the organism.

Different types of vectors can be used for transduction or transformation of animal cell, fungal cell or protist cell, preferably animal cells and more preferably human cells.

These include plasmid or viral vectors. Retroviral vectors have been used widely so far in gene therapy, particularly those based on Moloney murine leukemia virus (MoMLV), a member of the murine oncoretroviruses. Other murine retroviral vectors that can be used include those based on murine embryonic stem cell virus (MESV) and murine stem cell virus (MSCV). Vectors based on murine oncoretroviruses can be used for high efficiency transduction of cells, however, they require that the cells be active in cell division. Following entry into the cell cytoplasm and reverse transcription, transport of the preintegration complex to the nucleus requires the breakdown of the nuclear membrane during mitosis. Transduction of HP cells with murine retroviral based vectors therefore requires activation of the cells.

Lentiviral vectors, a subclass of the retroviral vectors, can also be used for high-efficiency transduction (Haas et al., Mol Ther 2:71-80 (2000); Miyoshi et al., Science 283:682-686 (1999); Case et al., Proc Natl Acad Sci USA 96:2988-2993 (1999)) and are able to transduce non-dividing cells. The preintegration complex is able to enter the nucleus without mitosis, and therefore lentiviral transduction does not require the induction of cells into cell cycle. This increases the likelihood that the cells remain pluripotent. Other groups of retroviruses such as spumaviruses, for example the foamy viruses, are also capable of efficiently transducing non-dividing cells.

Other types of viral vectors that can be used in the invention include adenoviral vectors (for example Fan et al., Hum Gene Ther 11:1313-1327 (2000); Knaan-Shanzer et al., Hum Gene Ther 12:1989-2005 (2001); Marini et al., Cancer Gene Ther 7:816-825 (2000)), adeno-associated viral (AAV) vectors (for example Fisher-Adams et al., Blood 88:492-504 (1996)), SV40 based vectors (for example Strayer et al., Gene Ther 7:886-895 (2000)), or forms of hybrid vectors (for example Feng et al., Nature Biotechnol 15:866-870 (1997) or Lieber et al., J Virol 73:9314-9324 (1999)). Adenoviral vectors can be readily produced at high titers, that can be easily concentrated (10¹² pfu/ml), and can transduce non-dividing cells. Large DNA inserts can be accommodated (7-8 kb). Immune reactions against adenovirus in vivo can be alleviated by removing genes encoding certain proteins.

AAV vectors are non-pathogenic, transduce both proliferating and nonproliferating cells, and integrate stably into the cellular genome (for example Grimm and Kleinschmidt Hum Gene Ther 10:2445-2450 (1999)). Moreover, they do not induce a host immune response and can be produced in helper-free systems to high titers of about 10¹⁰ cfu per ml. AAV is a non-enveloped virus with a single-stranded DNA genome. AAV vectors can ready incorporate up to about 4 kilobases of new DNA, although recent studies have extended this.

Vectors which result in integration of the introduced gene into the cell genome are preferred, for example retroviral vectors including lentiviral vectors, and AAV vectors. Integrating viral vectors are herein defined as those which result in the integration of all or part of their genetic material into the cellular genome. They include retroviral vectors and AAV vectors. They also include hybrid vectors such as adenoviral/retroviral vectors and adenoviral/AAV vectors. However, vectors that replicate stably as episomes can also be used. It is also desired that the vector can be produced in cell lines to a high titre, in a cost-effective manner, and have minimal risk for patients, for example not giving rise to replication competent virus. Retroviral vectors may be packaged in packaging cell lines such as the PA317 or AM-12 cell lines which contain helper vector(s) that is itself defective in packaging. Variations in the methods for producing high-titer retroviral supernatants include variations in the medium, packaging cells, temperature of harvest and concentration methods by centrifugation or complexation.

Retroviruses packaged in murine amphotropic envelopes may not transduce some cells efficiently due to low levels of the amphotropic receptor. However, cell cycle induction has been shown to lead to increased expression of the amphotropic receptor with a concordant increase in gene transfer. An alternative approach is to pseudotype retroviral vectors with envelopes such as the envelope from gibbon ape leukemia virus (GALV), vesicular stomatitis virus (VSV-G protein) or feline endogenous virus. Pseudo-typing vectors may allow concentration, for example by centrifugation.

AAV vectors may be produced in packaging cell lines or cells expressing the AAV rep and cap genes either constitutively or transiently. Production of AAV vectors has been aided by the development of helper-free packaging methods and the establishment of vector producer lines. Adenoviral vectors can be produced and purified according to standard methods known in the art.

Introduction of chimeric genes (or nucleic acid molecules) into the host cell can be accomplished by a variety of methods including calcium phosphate transfection, DEAE-dextran mediated transfection, electroporation, microprojectile bombardment, microinjection into nuclei and the like.

In another embodiment of the invention there is provided a transgenic, nonhuman animal, fungus or protist comprising cells having a chimeric nucleic acid molecule or a chimeric DNA molecule as hereinbefore described. The present invention also provides the use of a chimeric nucleic acid molecule or a chimeric DNA molecule as hereinbefore described for down regulating the expression of a target gene in a cell of an animal, fungus or protist.

A further aspect of the invention is a method of producing a transgenic, non-human animal wherein expression of a target gene in cells of the animal is down regulated, the method comprising the steps of

(a) providing a chimeric nucleic acid molecule or a chimeric DNA molecule as hereinbefore described to at least one cell of the animal;
(b) growing or regenerating a transgenic, non-human animal from said at least one cell of the animal.

The invention also provides a method of producing a transgenic fungal or protest organism wherein expression of a target gene in cells of the organism is down regulated, the method comprising the steps of:

(a) providing a chimeric nucleic acid molecule or a chimeric DNA molecule as hereinbefore described to at least one cell of the organism;
(b) growing or regenerating a transgenic organism from said at least one cell of the organism.

Transgenic animals can be produced by the injection of the chimeric genes into the pronucleus of a fertilized oocyte, by transplantation of cells, preferably uindifferentiated cells into a developing embryo to produce a chimeric embryo, transplantation of a nucleus from a recombinant cell into an enucleated embryo or activated oocyte and the like.

Methods for the production of trangenic animals are well established in the art and include U.S. Pat. No. 4,873,191; Rudolph et al. 1999 (Trends Biotechnology 17:367-374); Dalrymple et al. (1998) Biotechnol. Genet. Eng. Rev. 15: 33-49; Colman (1998) Bioch. Soc. Symp. 63: 141-147; Wilmut et al. (1997) Nature 385: 810-813, Wilmute et al. (1998) Reprod. Fertil. Dev. 10:639-643; Perry et al. (1993) Transgenic Res. 2: 125-133; Hogan et al. Manipulating the Mouse Embryo, 2nd ed. Cold Spring Harbor Laboratory press, 1994 and references cited therein.

The methods and means described herein, can be applied to any animal cell, fungal cell or protist cell in which gene-silencing takes place, including but not limited to invertebrate animals (such as insects, shellfish, molluscs, crustaceans such as crabs, lobsters and prawns) vertebrate animals (fish, avian animals, mammals, primates, humans) including domestic and farm animals, zoo or pet animals, mammals including mouse, rat, rabbit, pig, sheep, goat and cattle, yeast and fungi amongst others. The animal cell or organism may be a rodent, ovine, bovine, porcine, equine, canine, feline, ruminant or avian cell or organism. In particular embodiments, the cell is a human cell.

In a further aspect of the invention there is provided a composition comprising a chimeric nucleic acid molecule or a chimeric DNA molecule as hereinbefore described and a pharmaceutically acceptable carrier.

Another aspect of the invention provides a method of preparing a medicament for the treatment of an animal disease, comprising the composition of the invention.

The invention also provides a method of treating or preventing a disease in an animal, the method comprising administering a composition of the invention to an animal in need thereof.

A further aspect of the invention provides use of the composition of the invention in the preparation of a medicament for treating an animal disease.

The invention also provides compositions of the chimeric nucleic acids or chimeric genes with pharmaceutically acceptable carriers. The chimeric nucleic acids may be used in the form of pharmaceutical preparations which may be administered orally, for example in the form of tablets, coated tablets, capsules, solutions, emulsions or suspensions, or rectally, for example in the form of suppositories, or parenterally, for example in the form of injection solutions, or topically or locally, or with the aid of a catheter, or by inhalation, injection or infusion. Pharmaceutical preparations may be produced by processing the chimeric nucleic acids or chimeric genes in therapeutically inert organic and inorganic carriers. Examples of such carriers for tablets, coated tablets and capsules are lactose, corn starch or derivatives thereof, talc and stearic acid or salts thereof. Carriers suitable for the preparation of solutions include water, buffered salt solutions such as, for example, Hank's solution or Ringer's solution, polyols, solutions comprising sucrose, glucose or other sugars. Carriers suitable for injection include water, buffered salt solutions, alcohols, polyols, glycerol and vegetable oils. Carriers suitable for suppositories include oils, waxes, fats and semisolid polyols. The pharmaceutical preparations may also contain solvents, diluents, buffers, preservatives, thickeners, stabilizers, emulsifiers, wetting agents or surface active agents, liposomes or lipids, sweeteners, colorants, flavorings, osmotic agents, coating agents, or antioxidants. The chimeric nucleic acid or chimeric gene is preferably in a physiologically acceptable buffer which includes pharmaceutically acceptable salts, esters, or salts of such esters, which do not impair the biological activity of the compounds.

The compositions of the invention may comprise other therapeutically active substances such as drugs, antibodies, cytokines, antimicrobial agents, anti-inflammatory agents, anaesthetics, interferons and the like.

The invention also provides compositions of cells from an animal, fungus or protist, preferably animal cells and more preferably human cells, comprising the chimeric nucleic acid molecules or chimeric genes or vectors comprising the chimeric genes. The cells may be primary cells or cultured cells. The cells may be ex vivo or in vitro. The cells may be in vivo in an organism, preferably a non-human organism, more preferably a transgenic animal other than a human.

In a further embodiment, the invention provides a method of preparing a medicament comprising the chimeric nucleic acid molecules or chimeric genes or vectors comprising the chimeric genes. The vector may be a viral vector such as, for example, a retroviral vector which may be a lentiviral vector, adenoviral vector, adenovirus associated viral (AAV) vector or other viral vector.

A further aspect of the present invention provides a research reagent or kit comprising a nucleic acid vector for use in preparing a chimeric nucleic acid molecule or comprising a chimeric DNA molecule as hereinbefore described.

The invention also provides a package comprising the research reagent or kit described above and instructions for use thereof.

The invention also provides a library of chimeric genes comprising multiple individual chimeric genes, each being different, wherein each individual chimeric gene encodes a chimeric nucleic acid molecule or comprises a chimeric DNA molecule as hereinbefore described.

The invention also provides libraries of related chimeric nucleic acid molecules or chimeric genes as described herein, wherein individual members of the library comprise different antisense regions, each of which may be complementary to nucleotide sequences from transcribed nucleotide sequence of the same or different target genes. Alternatively, the members of the library may comprise the same antisense region and vary in a region comprising a largely double stranded nucleic acid sequence. The libraries may be readily constructed in bacterial host cells by inserting cDNA from a organism, preferably an animal cell, into a vector comprising a nucleotide sequence that, when transcribed, produces a transcript comprising a largely double stranded region, such that the cDNA sequence is operably linked to the nucleotide sequence. The library may comprise at least 100, 1000, or 5000 individual clones. The libraries may be introduced individually or en masse into cells for screening and identification of members that are capable of down regulating the expression of a target gene of interest. It will be apparent that such libraries are useful for functional genomics, for example, for the identification of genes associated with a phenotype of interest in a cell. The invention also provides methods of using such libraries.

The chimeric nucleic acid molecules, chimeric genes and libraries of the invention are also useful as research reagents or diagnostics. For example, the molecules and genes which are able to down regulate gene expression with specificity may be used by those of ordinary skill to elucidate the function of particular genes, for example to determine which genes confer or are involved in particular phenotypes in cells, or which viral genes are essential for replication, or to distinguish between the functions of various genes of a biological pathway. Since the chimeric nucleic acids of this invention hybridize to RNA or DNA from the target gene of interest, assays utilizing a hybridisation step can easily be developed to exploit this fact. Hybridization may be readily detected by enzyme conjugation, radiolabelling or any other suitable detection system. The invention therefore provides research reagents or kits or diagnostic kits comprising the chimeric nucleic acid molecules, chimeric genes or vectors required for producing these. The invention further provides packages which comprise such reagents or kits and instructions for their use.

The following non-limiting Examples describe method and means for enhanced antisense RNA mediated silencing of the expression of a target gene in a cell or combined sense/antisense RNA mediated target gene silencing.

Unless stated otherwise in the Examples, all recombinant DNA techniques are carried out according to standard protocols as described in Sambrook et al. (1989) Molecular Cloning: A Laboratory Manual, Second Edition, Cold Spring Harbor Laboratory Press, NY and in Volumes 1 and 2 of Ausubel et al. (1994) Current Protocols in Molecular Biology, Current Protocols, USA. Standard materials and methods for plant molecular work are described in Plant Molecular Biology Labfax (1993) by R. D. D. Croy, jointly published by BIOS Scientific Publications Ltd (UK) and Blackwell Scientific Publications, UK. Other references for standard molecular biology techniques include Sambrook and Russell (2001) Molecular Cloning: A Laboratory Manual, Third Edition, Cold Spring Harbor Laboratory Press, NY, Volumes I and II of Brown (1998) Molecular Biology LabFax, Second Edition, Academic Press (UK). Standard materials and methods for polymerase chain reactions can be found in Dieffenbach and Dveksler (1995) PCR Primer: A Laboratory Manual, Cold Spring Harbor Laboratory Press, and in McPherson at al. (2000) PCR-Basics: From Background to Bench, First Edition, Springer Verlag, Germany.

Throughout the description and Examples, reference is made to the following sequences:

SEQ ID No 1: oligonucleotide primer for the amplication of the RG1 PSTVd
SEQ ID No 2: oligonucleotide primer for the amplication of the RG1 PSTVd
SEQ ID No 3: nucleotide sequence of the genome of PSTVd RG1
SEQ ID No 4: nucleotide sequence of genome of the Australian Grapevine Viroid
SEQ ID No 5: nucleotide sequence of the genome of the Coconut Tinangaja Viroid
SEQ ID No 6: nucleotide sequence of the genome of the Tomato Manta Macho Viroid
SEQ ID No 7: nucleotide sequence of the genome of the Hop Latent Viroid
SEQ ID No 8: nucleotide sequence of the genome of the Tomato Apical Stunt Viroid
SEQ ID No 9: nucleotide sequence of the pdk2 intron
SEQ ID No 10: pTSVd sequence in pMBW491
SEQ ID No 11: pTSVd sequence in pMBW489 (with 10 nt deletion).

Throughout this specification the word “comprise”, or variations such as “comprises” or “comprising”, will be understood to imply the inclusion of a stated element, integer or step, or group of elements, integers or steps, but not the exclusion of any other element, integer or step, or group of elements, integers or steps.

All publications mentioned in this specification are herein incorporated by reference. Any discussion of documents, acts, materials, devices, articles or the like which has been included in the present specification is solely for the purpose of providing a context for the present invention. It is not to be taken as an admission that any or all of these matters form part of the prior art base or were common general knowledge in the field relevant to the present invention as it existed in Australia or elsewhere before the priority date of each claim of this application.

It will be appreciated by persons skilled in the art that numerous variations and/or modifications may be made to the invention as shown in the specific embodiments without departing from the spirit or scope of the invention as broadly described. The present embodiments are, therefore, to be considered in all respects as illustrative and not restrictive. In order that the nature of the present invention may be more clearly understood, preferred embodiments thereof will now be described with reference to the following non-limiting examples.

### EXAMPLES

### Example 1

### Construction of Different Chimeric Genes for Mediating Gene Silencing of a GFP Gene in Mammalian Cells and Analysis in CHO Cells

A gene encoding green fluorescent protein (GFP) with a “humanized” coding region was chosen as an example target gene for down-regulation of expression in mammalian cells. The construct pCi-GFP was obtained from Fiona Cameron of CSIRO Molecular Science. The GFP coding region was excised from pCi-GFP with NotI/NheI, blunted with Pfu polymerase to fill in the single-stranded ends, and inserted into the NheI site of the pCi vector after treatment with Pfu polymerase. The resultant plasmids were pMBW449 (also designated “asGFP”) which has the GFP coding region in an antisense orientation with respect to the CMV promoter of pCi, and the plasmid pMBW450 (also designated “senseGFP” or “sGFP”) which has the GFP coding region in the sense orientation with respect to the promoter. Both constructs have an SV40 nucleotide sequence comprising a polyadenylation signal following the GFP coding region. pMBW450 was used as the target gene construct in following experiments.

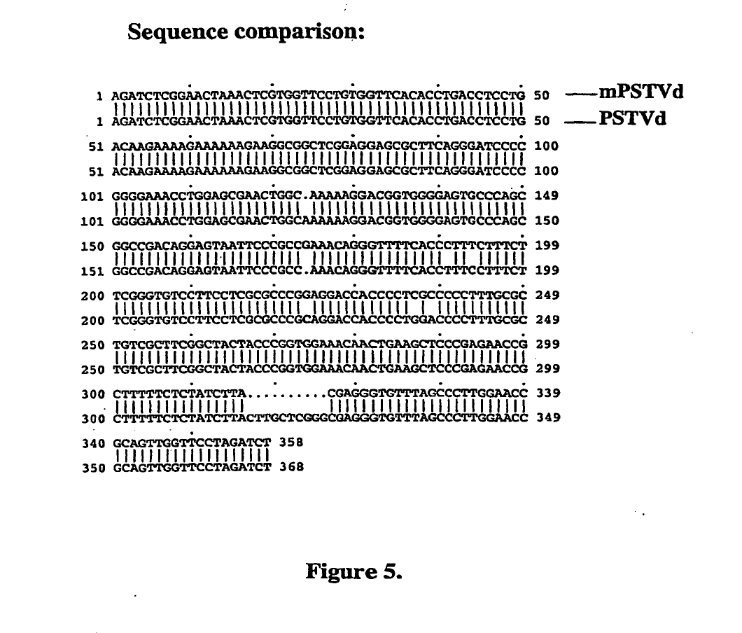

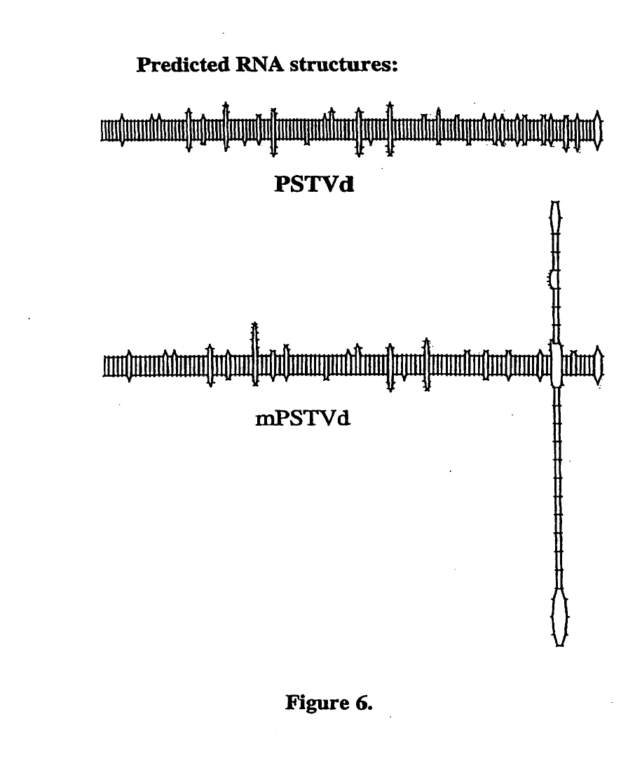

pMBW449 and pMBW450 were used as the base vectors for introduction of nucleotide sequences from PSTVd or a CUG repeat sequence, as follows. A full length sequence of the PSTVd strain RG1 (SEQ ID No 3) was amplified from a cDNA using oligonucleotides with the nucleotide sequence of SEQ ID No 1 or SEQ ID No 2. Several clones were obtained, including colony 1-9 which comprised nucleotides having the nucleotide sequence shown in FIG. 5 (“PSTVd”), and colony 1-4 which comprised nucleotides having the sequence shown in FIG. 5 (“mPSTVd”). The colony 1-9 sequence of 368 nucleotides differed from the wild-type PSTV sequence of strain RG1 at positions as follows: lacking a G nucleotide between positions 174 and 175 of the 1-9 sequence, a T at position 191 rather than C, a G at position 236 rather than C, an A at position 238 rather than C, lacking a CG dinucleotide between positions 322 and 323, and having an AGATCT sequence for the restriction enzyme BglII at each end. The colony 1-4 sequence differed from the colony 1-9 sequence primarily in the presence of a 10 nucleotide deletion, which had arisen spontaneously, corresponding to nucleotides 316-325 of the colony 1-9 sequence. This resulted in an alteration in the predicted RNA structure for the mPSTVd RNA sequence compared to the PSTVd RNA sequence (FIG. 6). The rod-like structure of PSTVd was altered to a cruciform-like structure.

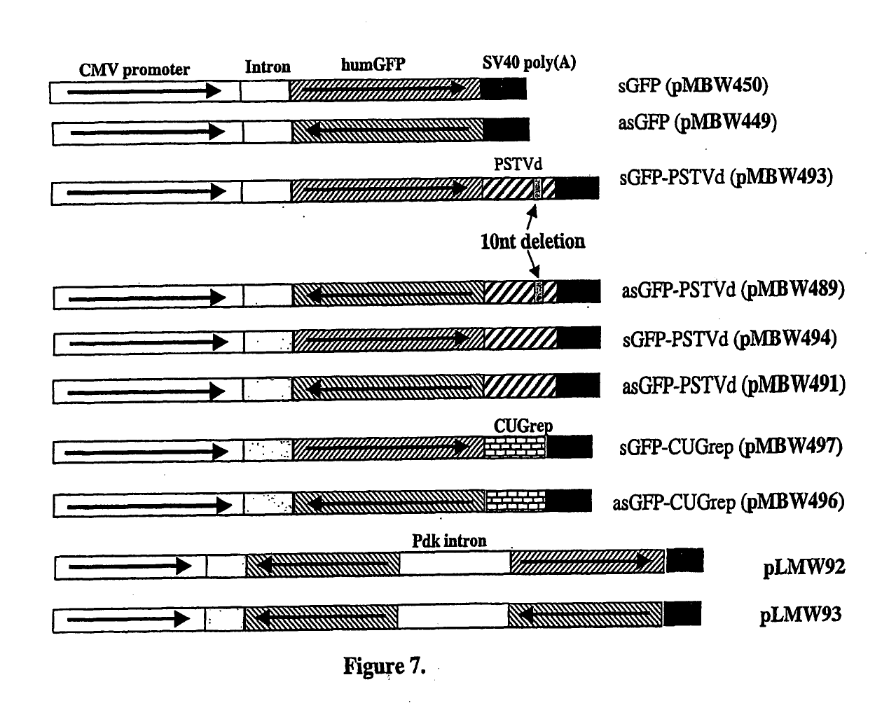

The PSTVd sequences were excised from DNA from the two plasmid clones with EcoRI and inserted into the EcoRI sites of pMBW449 or pMBW450. The resultant plasmids were pMBW491 (“asGFP-PSTVd”), pMBW494 (“sGFP-PSTVd”), pMBW489 (“asGFP-mPSTVd”) and pMBW493 (“sGFP-mPSTVd”). These are shown schematically in FIG. 7. That is, the GFP coding region was in a sense orientation in pMBW493 and pMBW494, and in an antisense orientation in pMBW489 and pME3W491 with regard to the CMV promoter region. Of these, plasmids pMBW493 and pMBW489 contained downstream of the GFP coding region, but upstream of the SV40 polyadenylation signal, the nucleotide sequence corresponding to a PSTVd sequence but with a 10 nt deletion (SEQ ID No 11). Plasmids pMBW494 and pMBW491 contained downstream of the GFP coding region, but upstream of the SV40 polyadenylation signal, a nucleotide sequence corresponding to a PSTVd sequence (SEQ ID No 10) without the 10 nt deletion.

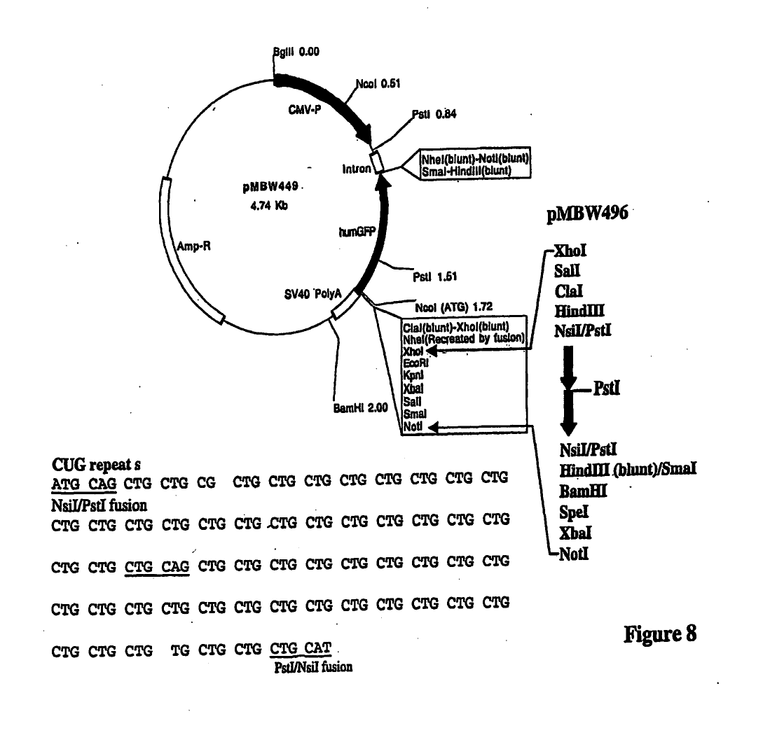

A nucleotide sequence encoding 54 CUG trinucleotide repeats was synthesized using oligonucleotides, forming pMBW451. The plasmid DNA sequence of the region encoding the CUG trinucleotide repeats (CTG in DNA) is shown in FIG. 8. The CUG repeat sequence was excised from pMBW451 with XhoI/NotI and inserted into the XhoI/NotI site of pMBW449 and pMBW450 to form pMBW496 (“asGFP-CUGrep”) and pMBW497 (“sGFP-CUGrep”), respectviely. Plasmids pMBW497 and pMBW496 therefore contained downstream of the GFP coding region, but upstream of the SV40 polyadenylation signal, a nucleotide sequence comprising 54 CUG trinucleotide repeats.

The different plasmids were introduced at a series of concentrations (0.1, 0.3, 0.5, 0.7 μg per well) into CHO cells in combination with pMBW450 as the target gene construct. Since the GFP sense constructs pMBW493, pMBW494 and pMBW497 contain a functional GFP sequence, these constructs were also introduced into separate samples of cells in the absence of pMBW450 in order to estimate the GFP expression by these constructs alone. As a further control, pMBW450 was introduced alone into CHO cells.

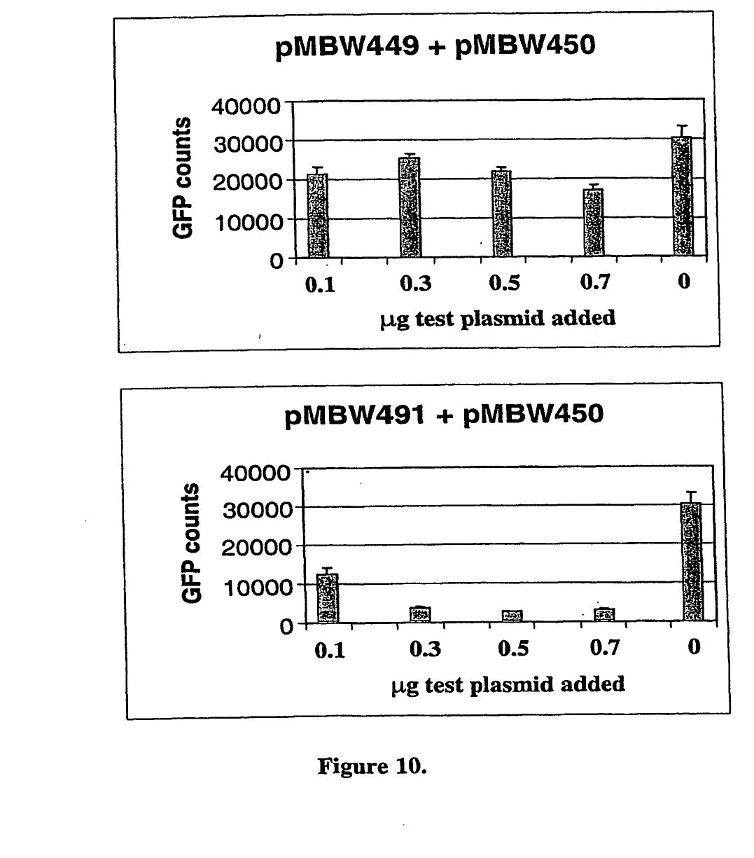

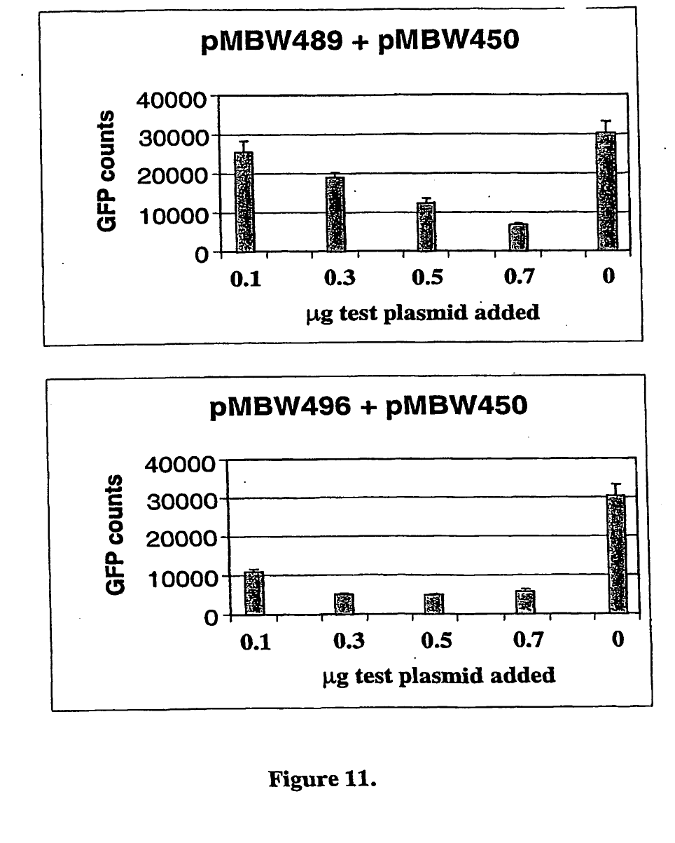

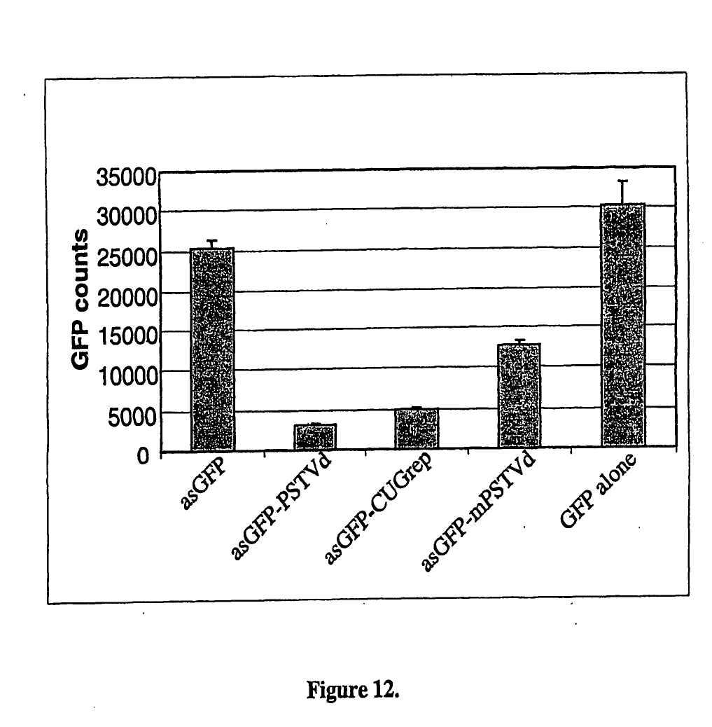

After 24 hrs or 48 hrs, the cells were assayed for GFP expression. Average counts and standard deviations are represented in FIGS. 10 and 11. The antisense GFP construct pMBW449 caused only a slight reduction in GFP expression (FIG. 10, top panel). However, pMBW491, pMBW496 and pMBW489 that comprise a PSTVd or CUG repeat sequences joined to the antisense GFP sequence caused a significantly enhanced reduction of the expression of the GFP gene (FIG. 10, lower panel and FIG. 11). The extent of reduction was generally dose-responsive, that is, correlated well with the amount of effector plasmid added. The extent of effect caused by each construct could be compared at the 0.3 μg level, as shown in FIG. 12. The most effective was pMBW491, followed by pMBW496. Interestingly, addition of pMWB489 in which the PSTVd sequence contained a 10 nt deletion, resulted in a slower and lesser extent of GFP silencing than pMWB491, which contained a complete PSTVd sequence.

### Example 2

### Use of Chimeric Nucleic Acid Molecules for Mediating Gene Silencing of a GFP Gene in Mammalian Cells—Cancer Cells

To compare the efficiency of gene silencing of the modified antisense to a hairpin RNA (RNAi) construct, we constructed pLMW90 as follows. A Flaveria pdk intron sequence obtained from pHannibal was excised with EcoRI/XbaI and inserted into the EcoRI/XbaI site of pMBW449, giving pLMW90. This plasmid already contained an antisense GFP sequence. A second GFP sequence was inserted, orientated in a sense direction with respect to the promoter, by inserting a GFP fragment excised with NheI/SmaI and treated with Pfu polymerase to blunt the fragment, into the SmaI site of pLMW90, forming the hairpin RNA construct pLMW92 which contained an inverted repeat (antisense/sense) of the GFP sequence separated by the pdk intron, and pLMW93 which contained a direct repeat of the antisense GFP sequence (antisense/antisense). These constructs are shown schematically in FIG. 7.

### Transfection Method:

Day 1: 2× 96-well TC trays were seeded with cells at 2×10⁴ cells per well in appropriate medium. For HT29 cells (Human Colon Carcinoma; ATCC #HTB-38) RPMI medium (Gibco Cat No: 31800-014) was used, supplemented with 10% FCS and 2 mM L-Glutamine. For CHO (Chinese Hamster Ovary) cells, EMEM (Trace Cat No: 50-011-PA) was used, supplemented with 10% FCS and 1 mM Sodium Pyruvate. Cells were incubated overnight at 37° C. in 5% CO₂.

Day 2: Cells were approximately 50% confluent at time of transfection. Plasmid pCi-Gal (a negative control plasmid) was transfected alone, at 1.0 g per well in six replicate wells across duplicate plates.

pMBW450 (positive control) was transfected alone, at 0.3 g per well, in six replicate wells across duplicate plates.

Other DNAs (pMBW449, pMBW489, pMBW491, pMBW493, pMBW496, pMBW497, pLMW92, pLMW93, pMBW512 and pMBW513-silencing DNA's), were transfected individually, using increasing amounts of DNA, namely 0.1, 0.3, 0.5 and 0.7 g with 0.3 g pMBW450 per well (target DNA), in six replicate wells across duplicate plates. All DNA concentrations were made up to 1.0 g per well using pCi-Gal carrier DNA. Cationic lipid CS087 or CS102 was used as the transfection agent at 21M per well. All transfections were carried out using Serum-free (SF) medium. All plasmids were diluted to 20 ng/μl in sterile purified water. Appropriate amounts of target DNA, silencing DNA and carrier DNA were added to the wells of a 96 well polystyrene dilution tray to give a total of 2.2 g per well in 110 l (enough for 2×1.0 g transfections plus 10%). 110 l of lipid diluted to 42M in SF medium was then added to each well and the DNA:lipid complexes allowed to form at room temperature for 10 minutes. The cells were washed with SF-medium and 2×100 l aliquots of DNA:lipid was added to the corresponding wells of the duplicate trays of cells. The cells were incubated for 4 hours, then 100 l of medium containing 20% FCS was added to each well. The cells were incubated overnight.

Day 3: The cells were washed 2× with 100 l PBS. 100 l of lysis buffer (250 mM Tris pH8.0, 0.1% Triton X-100) was added to each well. The plates were wrapped in parafilm and frozen overnight at −20° C. to induce cell lysis.

Day 4: The plates were thawed at room temperature for 30 minutes. An 801 aliquot was taken from each well and transferred to a black 96 well plate. The GFP counts were read on a Wallac Victor² 1420 Multilabel Counter Plate reader. The average of the pCi-Gal negative control wells was subtracted from all readings. The averages of the 6 replicate wells were calculated and normalized to zero (lowest negative value was added to all values so lowest value became zero) and plotted with their standard deviations using Excel software.

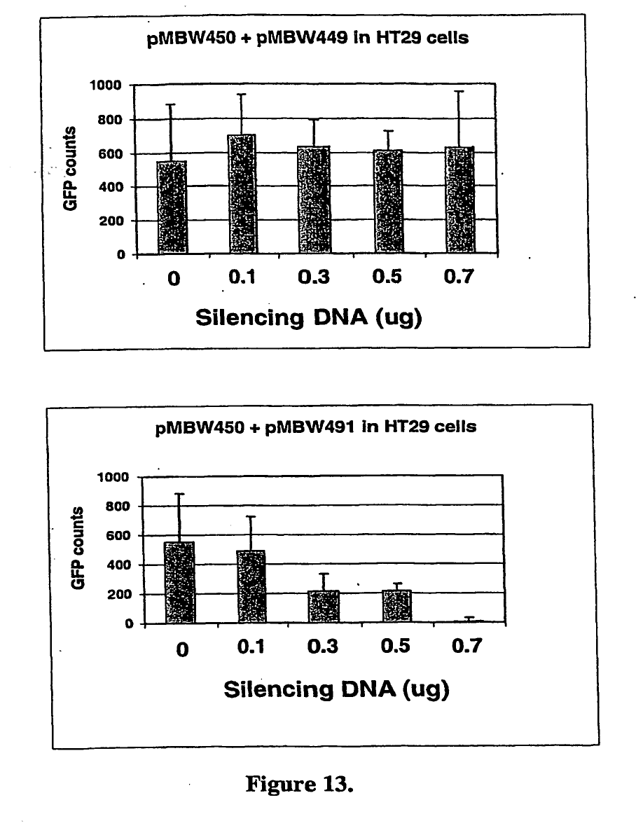

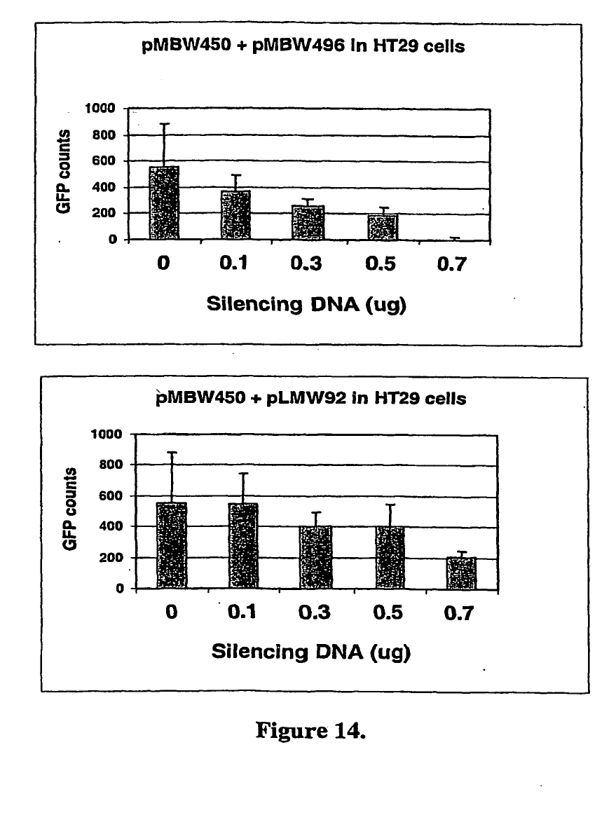

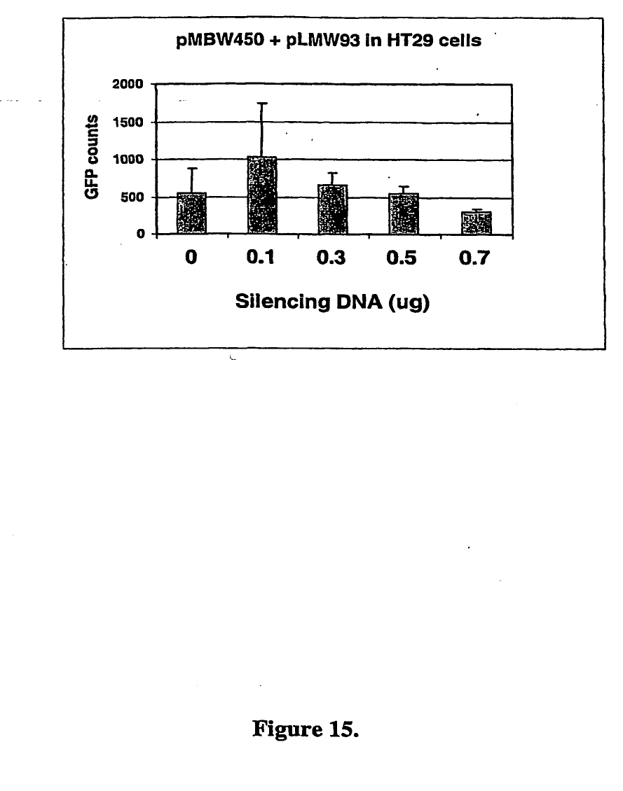

The results are shown in FIGS. 13-15. The antisense constructs pMBW449 and pLMW93 caused only slight or insignificant silencing. In contrast, both pMBW491 (PSTVd sequence) and pMBW496 (CUGrep) were highly effective in silencing, achieving greater than 90% reduction in GFP gene expression at the highest level of effector plasmid added. Both of these constructs were more effective than the hairpin RNA construct pLMW92, which was moderately effective (FIG. 14, lower panel).

### Example 3

### Assessment of Gene Silencing in Animal Cells

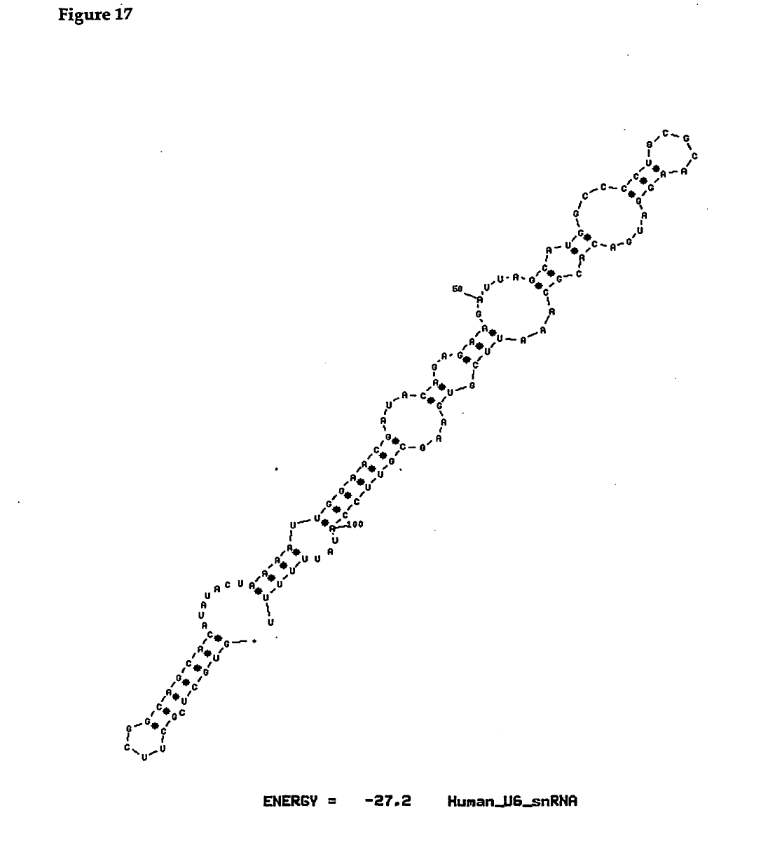

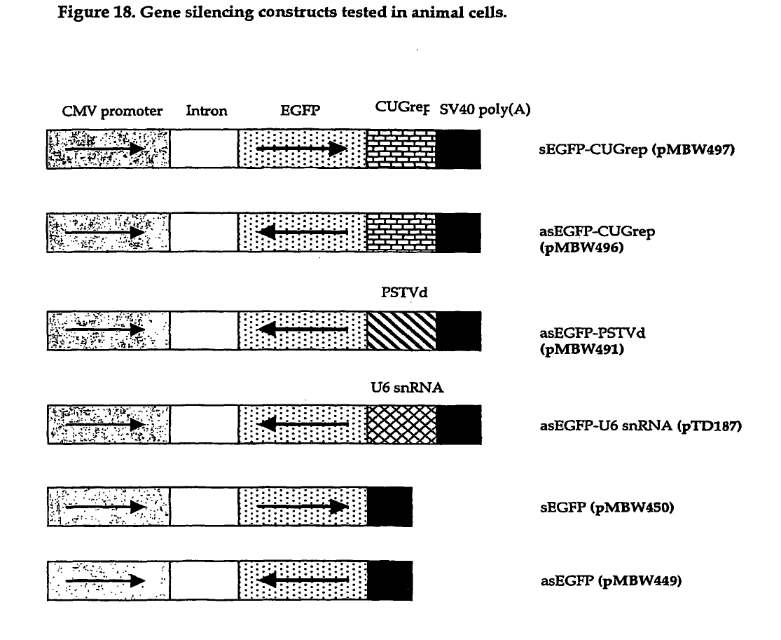

To determine whether gene silencing could be enhanced in a range of animal cells by the use of the modified nucleic acid molecules, experiments were carried out to silence a reporter gene (EGFP) in a variety of animal cell types using the gene silencing constructs shown schematically in FIG. 18. A further construct was made for comparison with the others, in order to test a region from an snRNA as a nuclear localisation signal. Small nuclear RNA (snRNA) molecules are known to be nuclear localised and to include largely double stranded regions. An example of an snRNA is the U6 RNA molecule. The sequence of the human U6 RNA is shown in FIG. 16, and shown schematically as a folded structure in FIG. 17 (lowest predicted free energy).

The PSTVd sequence on pMBW491 was replaced with a sequence from the human U6 snRNA. The human U6 snRNA was amplified by PCR from genomic DNA isolated from cultured HeLa cells, using:

(SEQ ID NO: 15)
forward primer (U6MunF)
5′-TATGCACAATTGGTGCTCGCTTCGGCAGC- 3′;
and
(SEQ ID NO: 16)
reverse primer (UGMunR)
5′-TGCACCCAATTGTATGGAACGCTTCACGAA-3′.

Both primers contain MunI restriction sites (CAATTG), which were used to replace the PSTVd sequence on pMBW491 with the amplified U6 snRNA sequence. The PSTVd sequence was removed from pMBW491 by deletion of an EcoRI restriction fragment. MunI and EcoRI have compatible cohesive overhangs, and so enabled the insertion of U6 snRNA into the EcoRI site on pMBW491. The resulting plasmid was named pTD187 and is shown schematically in FIG. 18. Expression of the chimeric gene in cells from the CMV promoter resulted in production of a chimeric RNA comprising an antisense sequence to the EGFP gene joined to the U6 RNA, with the antisense sequence 5′ to the U6 sequence.
Co-Transfection of Animal Cells with Gene Constructs and pEGFP-N1

The animal cell types used with the gene silencing constructs were from the cell lines HeLa (human), Vero (monkey), MDCK (dog), L929 (mouse), ST (pig), MDBK (cow) and CHSE (fish) (see Table 1). The gene silencing plasmids (1 μg) were co-transfected with the EGFP reporter plasmid pEGFP-N1 (Clonetech) (1 μg) into the cells using either Lipofectamine 2000 (Invitrogen) (HeLa, Vero and ST), or electroporation with a Nucleofector electroporator (Amaxa) (MDCK, L929, MDBK and CHSE) according to the manufacturers instructions. Each transfection was performed in triplicate. Once transfected, the cells were grown in 24 well plates. 48 hours post-transfection, cells were scraped from the wells and resuspended in FACS wash buffer (PBS with 0.05% sodium azide). EGFP fluorescence was then measured using a Becton Dickinson FACScalibar detecting EGFP in fluorescence parameter FL1. Silencing of EGFP by the gene silencing plasmids was calculated relative to the expression level obtained in the presence of the plasmid pMBW497. The mean fluorescence intensity of EGFP in cells transfected with pMBW497 and pEGFP-N1 was normalised to 100% and the intensity of the EGFP signal obtained in the presence of the other plasmids was compared to this control. This gene construct was chosen as a control since it encodes a truncated (non-functional) EGFP gene and a nuclear localisation signal, and was therefore more similar to the test constructs.

TABLE 1
Percentage of EGFP silencing by gene silencing constructs
relative to pMBW497 control. Plasmids were tested in
triplicate and standard errors are shown.

| | pMBW496 | pMBW491 | pTD187 |
|---|---|---|---|
| Cell Line | (CUGrep) | (PSTVd) | (U6 snRNA) |
| HeLa (Human) | 55.02 +/− 1.57 | 41.33 +/− 0.82 | 76.23 +/− 0.79 |
| Vero (monkey) | 24.28 +/− 2.13 | 33.25 +/− 2.82 | 50.83 +/− 0.30 |
| MDCK (dog) | 38.46 +/− 0.49 | 38.26 +/− 1.99 | 30.85 +/− 0.97 |
| L929 (mouse) | 29.30 +/− 4.10 | −23.28 +/− 2.49 | 22.97 +/− 0.05 |
| ST (pig) | 36.71 +/− 4.40 | 16.35 +/− 5.85 | 55.34 +/− 2.59 |
| MDBK (cow) | 81.14 +/− 0.60 | 75.84 +/− 0.21 | 84.20 +/− 0.71 |
| CHSE (fish) | 86.18 +/− 0.32 | 83.81 +/− 0.12 | 81.37 +/− 0.27 |

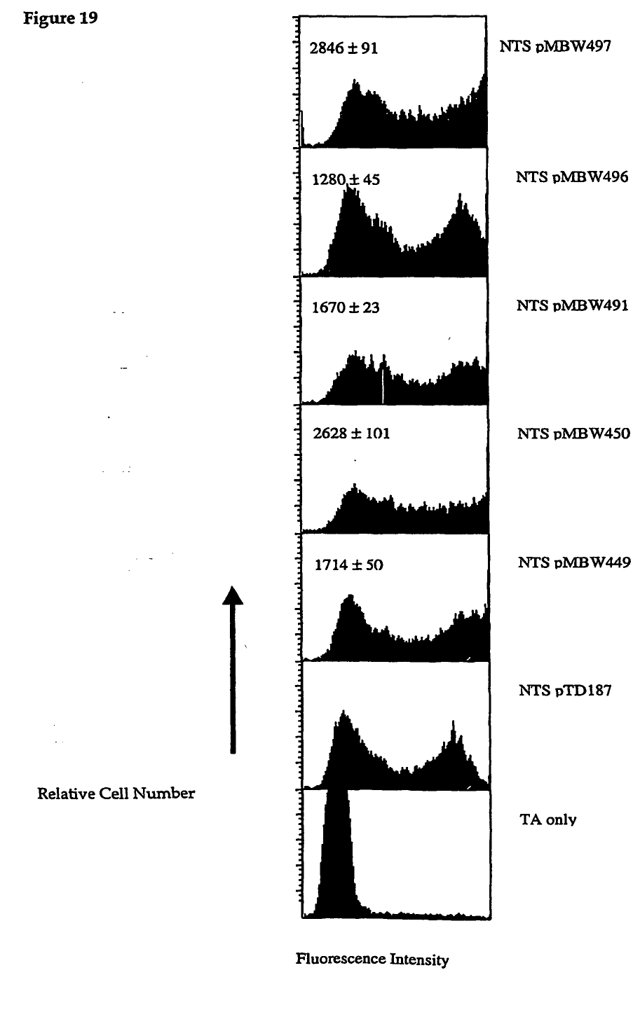

Representative results from the FACS analysis for silencing of EGFP in HeLa cells are shown in FIG. 19. The FACS histograms showed a shift in fluorescence intensity for the cell populations when a silencing construct was used. pMBW496 and pMBW491 encoding the EGFP antisense and CUG repeat or PSTVd sequences, respectively, both demonstrated silencing when compared to the pMBW497 control. pTD187 encoding the antisense sequence joined to the human U6 snRNA gave the greatest extent of silencing, approximately 80% silencing of EGFP compared to the control. The percentage of EGFP silencing by the constructs in the animal cells tested is shown in Table 1. The results were from a representative silencing experiment for each of the cell lines. Each gene silencing construct was tested in triplicate in each experiment and standard errors for each measurement are given in Table 1. These data show that each of the gene constructs was effective in silencing target gene expression in a variety of animal cell types, with the most effective being a construct encoding an RNA comprising an snRNA sequence.

### Example 4

### Gene Silencing of a Viral Gene

Influenza A virus replicates in the nucleus and is a good example of a viral target. Influenza A strain PR8 was amplified in chick embryos and adapted to MDCK cells. Adaptation of the virus was demonstrated by hemagglutination assays and by measurement of cytopathic effects on the cells.

### Construction of Gene Silencing Plasmids Targeting the Influenza A Nuclear Protein (NP) Gene.

Human beta-globin intron 1 was amplified by PCR from the mammalian expression plasmid pCI (Promega):

The forward primer was (CMVintF):

(SEQ ID NO: 17)
5′TCATCAGAATTCGCAGGTAAGTATCAAGG3′;

and
The reverse primer was (CMVintR):

| | |
|---|---|
| 5′TGGACAAGATATCGACACCTGTGGAGAGAA3′. | (SEQ ID NO: 18) |

The intron was inserted into the EcoRI and EcoRV sites of pTracer-CMV2 (Invitrogen) using the compatible restriction sites incorporated into the primers. This plasmid was named pTD162. Part of the NP gene sequence was then amplified by PCR from an existing cloned NP gene that was derived from Influenza A strain A/PR/8/34 (Accession number NC002019).
The forward primer was (HINUAPNPF):

(SEQ ID NO: 19)
5′ GATGCAGGTACCGCGGCCGCGAACTGAGAAGCAGGTAC3′;

and
The reverse primer was (HINUAPNPMPR):

(SEQ ID NO: 20)
5′ GATCTACAATTGCAGCTGTCCTTCATTACTCATGTC3′.

This fragment was cloned into the EcoRV and NotI sites of pTD162 using the PvuII and NotI sites incorporated into the primer sequences. The resulting plasmid was named pTD173. The PSTVd and U6 snRNA sequences were then cloned into the NotI and XbaI sites of pTD173 using NotI and NheI sites incorporated into the PCR primers. The PSTVd sequence was amplified from pMBW491, using the forward primer (NOTPSVF):

(SEQ ID NO: 21)
5′ TCAATGGCGGCCGCCGGAACTAAACTCGTGGTT3′;

and
reverse primer (PSVNHER):

(SEQ ID NO: 22)
5′ CAATAGGCTAGCAGGAACCAACTGCGGTTCC3′.

The U6 snRNA sequence was amplified from pTD187 using the forward primer (U6NOTF):

(SEQ ID NO: 23)
5′ TGAACTGCGGCCGCGTGCTCGCTTCGGCAGC3′;

and
reverse primer (U6NHER):

| | |
|---|---|
| 5′ ACCTGAGCTAGCTATGGAACGCTTCACGAA3′. | (SEQ ID NO: 24) |

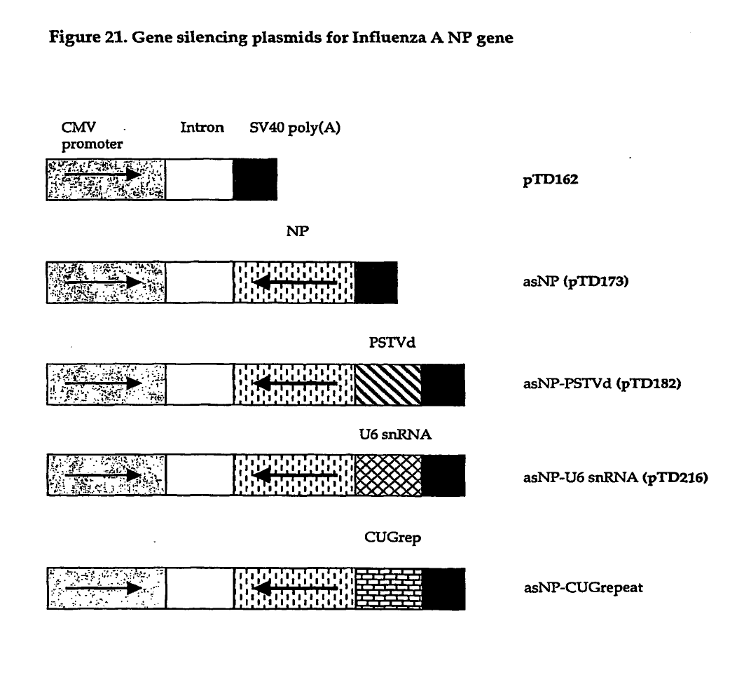

The NP-PSTVd plasmid was designated pTD182 and the NP-U6 snRNA plasmid was designated pTD216. Corresponding constructs for targeting the influenza NP gene containing an CUG repeat are made in the same way as for pTD182 and 216. These plasmids are shown schematically in FIG. 21.

These plasmids were introduced into MDCK cells using a Nucleofector Electroporator according to the manufacturers instructions, and were challenged with influenza A virus. Viral replication is measured by hemaglutinnation assays or by measuring cytopathic effects on the cells. Reduced levels of viral replication are seen in the presence of the gene silencing constructs targeting the viral gene.

### Example 5

### Lentivirus Constructs to Generate Transgenic Mice

Gene silencing constructs targeting the mouse β2-microglobulin gene (Accession No. NM009735) have been made. This gene was targeted as it is highly conserved between species and constitutively expressed by nearly all cells. Furthermore, well defined reagents for assaying the levels of expression of β2-microglobulin are commercially available. The region of the gene coding region between nucleotide positions 75 and 359 was amplified by PCR. This nucleotide sequence is joined in the antisense orientation to the nuclear localising nucleotide sequences in the expression vectors pTD 182 (PSTVd), pTD218 (U6 25 snRNA) and one containing a CUG repeat sequence. The resulting plasmids can be used to generate transgenic lentiviral transfer vectors as follows. The gene silencing expression cassettes are proposed to be amplified by PCR using primers that incorporate NruI restriction sites. The PCR fragments can then be blunt end cloned into a compatible restriction site in a lentiviral transfer vector. The vectors can be packaged into lentiviral particles by co-transfection of the lentiviral vector construct and packaging vectors into mouse 293T cells. Once lentivirus particles have been generated, a small volume of high titre virus is proposed to be infected into the perivitelline space of single-cell mouse embryos which will then be implanted into pseudo-pregnant female recipient mice.

Resulting neonates are proposed to be screened for integration of the lentivirus by Southern blotting and expression of lentivirus encoded GFP by fluorescence. Reduction of β2-microglobulin gene expression can then be assayed for at the protein level using specific antibodies to β2-microglobulin and at the mRNA level using quantitative (real-time) RT-PCR.

1. A method of down regulating the expression of a target gene in a cell of an animal, fungus or protist, the method comprising the step of providing the cell with a chimeric nucleic acid molecule, wherein the molecule comprises
a) a target-gene specific region comprising a nucleotide sequence of at least 16 consecutive nucleotides having 100% sequence identity with the complement to 16 consecutive nucleotides from a transcript of the target gene, and
b) a largely double stranded nucleic acid region of 60 to 360 nucleotides,
wherein the target gene is a reporter gene, a pathogenic animal virus gene, a cancer-related gene, an oncogene, an immunomodulatory gene, a gene encoding a cytokine, growth factor, enzyme or a transcription factor or an animal disease causing gene.

2. The method of claim 1, wherein the chimeric nucleic acid molecule is a RNA molecule.

3. The method of claim 1, wherein the cell is an animal cell.

4. The method of claim 1, wherein the largely double stranded nucleic acid region comprises a nuclear localization signal.

5. The method of claim 1, further comprising the step of identifying a cell of an animal, fungus or protist, wherein the expression of the target gene is down regulated.

6. The method of claim 1, wherein the largely double stranded nucleic acid region comprises a nucleotide sequence obtained from a small nuclear RNA (snRNA).

7. The method of claim 1, wherein the largely double stranded nucleic acid region comprises a nucleotide sequence obtained from a small nuclear RNA (snRNA) that is U3, U2, U4 to U6, U8, U13 to U16, U18 to U21, U23 to U72, 4.5S RNAI to III, 5S RNAIII, E2 or E3.

8. The method of claim 6, wherein the largely double stranded nucleic acid region comprises a nucleotide sequence obtained from a small nucleolar localised RNA (snoRNA).

9. The method of claim 6, wherein the largely double stranded nucleic acid region comprises a nucleotide sequence obtained from U6 snoRNA.

10. The method of claim 6, wherein the largely double stranded nucleic acid region comprises a nucleotide sequence obtained from human U6 snoRNA set forth in SEQ ID NO: 27.

11. The method of claim 1, wherein the largely double stranded nucleic acid region comprises a nucleotide sequence obtained from a viroid of the Potato Spindle Tuber Viroid (PSTVd)-type, a nucleotide sequence comprising at least 35 repeats of a trinucleotide CUG, CAG, GAC or GUC, a nucleotide sequence obtained from hepatitis delta RNA, or a synthetic nucleotide sequence comprising a nucleic acid-nuclear localization signal.

12-17. (canceled)

18. The method of claim 11, wherein the largely double stranded nucleic region comprises a RNA sequence having at least 35 repeats of the trinucleotide CUG.

19. The method of claim 18 wherein the largely double stranded nucleic acid region comprises a RNA sequence having between 44 and 2000 repeats of the trinucleotide CUG.

20. The method of claim 1, wherein the chimeric nucleic acid molecule comprises multiple target-gene specific regions.

21. The method of claim 1, wherein the chimeric nucleic acid molecule comprises an intron sequence.

22. (canceled)

23. The method of claim 1, wherein the cell is from an animal that is a human, vertebrate, mammalian, fish, cattle, goat, pig, sheep, rodent, hamster, mouse, rat, guinea pig, rabbit, primate, nematode, shellfish, prawn, crab, lobster, insect, fruit fly, Coleapteran insect, Dipteran insect, Lepidopteran insect or Homeopteran insect.

24. The method of claim 1, wherein the chimeric nucleic acid is an RNA molecule produced by transcription of a chimeric DNA molecule.

25. A chimeric nucleic acid molecule for down regulating the expression of a target gene in a cell of an animal, fungus or protist, wherein the molecule comprises
a. a target-gene specific region comprising a nucleotide sequence of at least 16 consecutive nucleotides having sequence identity with the complement to 16 consecutive nucleotides from a transcript of the target gene; and
b. a largely double stranded nucleic acid region of 60 to 360 nucleotides,
wherein the target gene is a reporter gene, a pathogenic animal virus gene, a cancer-related gene, an oncogene, an immunomodulatory gene, a gene encoding a cytokine, growth factor, enzyme or a transcription factor or an animal disease causing gene.

26-55. (canceled)

56. A cell of an animal, fungus or protist comprising the chimeric nucleic acid molecule according to claim 25.

57-107. (canceled)

108. A method of down regulating the expression of at least two target genes in a cell of an animal, fungus or protist, the method comprising the step of providing the cell with a chimeric nucleic acid molecule, wherein the molecule comprises
a) at least two target-gene specific regions each comprising a nucleotide sequence of at least 16 consecutive nucleotides having 100% sequence identity with the complement to 16 consecutive nucleotides from a transcript of different target genes, and
b) a largely double stranded nucleic acid region of 60 to 360 nucleotides,
wherein each target gene is independently a reporter gene, a pathogenic animal virus gene, a cancer-related gene, an oncogene, an immunomodulatory gene, a gene encoding a cytokine, growth factor, enzyme or a transcription factor or an animal disease causing gene.


SEQUENCE LISTING (27 sequences)

SEQ ID NO: 1 - DNA, 29 bp, Artificial Sequence
  oligonucleotide primer for the PCR amplification of the genome of PSTVd RG1
cgcagatctcggaactaaactcgtggttc

SEQ ID NO: 2 - DNA, 27 bp, Artificial Sequence
  oligonucleotide primer for the PCR amplification of the genome of PSTVd RG1
gcgagatctaggaaccaactgcggttc

SEQ ID NO: 3 - DNA, 359 bp, Potato spindle tuber viroid
cggaactaaactcgtggttcctgtggttcacacctgacctcctgacaagaaaagaaaaaagaaggcggctcggaggagcgcttcagggatccccggggaaacctggagcgaactggcaaaaaaggacggtggggagtgcccagcggccgacaggagtaattcccgccgaaacagggttttcacccttcctttcttcgggtgtccttcctcgcgcccgcaggaccacccctcgccccctttgcgctgtcgcttcggctactacccggtggaaacaactgaagctcccgagaaccgctttttctctatcttacttgctccggggcgagggtgtttagcccttggaaccgcagttggttcct

SEQ ID NO: 4 - DNA, 369 bp, Australian grapevine viroid
tgggcaccaactagaggttcctgtggtactcaccgaaggccgcgaacgtaggaaagaaaaagatagaaaagctgggtaagactcacctggcgactcgtcgtcgacgaagggtcctcagcagagcaccggcaggaggcgctatgcaggaacgctaggggtcctccagcggaggactgaagaaactccggtttcttctttcactctgtagctggaatccctgttgcgcttgctggcgaaacctgcagggaagctagctgggtcccgctagtcgagcggactcgtcccagcggtcccaaccagttttctttatcctatttttcctgcgggcgcccggtcgtggttaccctggagctccctgtttggaggccc

SEQ ID NO: 5 - DNA, 254 bp, Coconut tinangaja viroid
ctggggaattcccacggctcggcaaaataaaagcacaagagcgactgctagagggatccccggggaaacccctagcaaccgaggtagggagcgtacctggtgtcgccgattcgtgctggttgggcttcgtcccttccgagcttcgatccgacgcccggccgcttcctcgccgaagctgctacggagactacccggtggatacaactctttgcagcgccctgtgtaataaaagctcgagtccggtttgcgcccct

SEQ ID NO: 6 - DNA, 360 bp, Tomato planta macho viroid
cgggatcttttccttgtggttcctgtggtacacacctgacctcctgaccagaaaagaaaaaagaattgcggccaaaggagcgcttcagggatccccggggaaacctggagcgaactggcgaaggagtcgcggctggggagtctcccagacaggagtaatccccgctgaaacagggttttcacccttcctttcttcgggtttccttcctctgcggtcgacaccctcgcccgcttctcttgcgctgtcgcttcggagactacccggtggaaacaactgaagctcccaagcgccgctttttctctatcttgctggctccggggcgagggtggaaaaccctggaacccttcgaaaagggtccct

SEQ ID NO: 7 - DNA, 256 bp, Hop latent viroid
ctggggaatacactacgtgacttacctgtatgatggcaagggttcgaagagggatccccggggaaacctactcgagcgaggcggagatcgagcgccagttcgtgcgcggcgacctgaagttgcttcggcttcttcttgttcgcgtcctgcgtggaacggctccttctccacaccagccggagttggaaactacccggtggatacaactcttgagcgccgagctttacctgcagaagttcacataaaaagtgcccat

SEQ ID NO: 8 - DNA, 360 bp, Tomato apical stunt viroid
cgggatctttcgtgaggttcctgtggtgctcacctgaccctgcaggcatcaagaaaaaagataggagcgggaaggaagaagtccttcagggatccccggggaaacctggaggaagtcgaggtcgggggcttcggatcattcctggttgagacaggagtaatcccagctgaaacagggttttcacccttcctttcttctggtttccttcctctcgccggaaggtcttcggccctcgcccggagcttctctctggagactacccggtggaaacaactgaagcttccacttccacgctctttttctctatctttgttgctctccgggcgagggtgaaagcccgtggaaccctgaatggtccct

SEQ ID NO: 9 - DNA, 786 bp, Artificial Sequence
  nucleotide sequence of the pdk2 intron
aagcttggtaaggaaataattattttcttttttccttttagtataaaatagttaagtgatgttaattagtatgattataataatatagttgttataattgtgaaaaaataatttataaatatattgtttacataaacaacatagtaatgtaaaaaaatatgacaagtgatgtgtaagacgaagaagataaaagttgagagtaagtatattatttttaatgaatttgatcgaacatgtaagatgatatactagcattaatatttgttttaatcataatagtaattctagctggtttgatgaattaaatatcaatgataaaatactatagtaaaaataagaataaataaattaaaataatatttttttatgattaatagtttattatataattaaatatctataccattactaaatattttagtttaaaagttaataaatattttgttagaaattccaatctgcttgtaatttatcaataaacaaaatattaaataacaagctaaagtaacaaataatatcaaactaatagaaacagtaatctaatgtaacaaaacataatctaatgctaatataacaaagcgcaagatctatcattttatatagtattattttcaatcaacattcttattaatttctaaataatacttgtagttttattaacttctaaatggattgactattaattaaatgaattagtcgaacatgaataaacaaggtaacatgatagatcatgtcattgtgttatcattgatcttacatttggattgattacagttgggaaagctt

SEQ ID NO: 10 - DNA, 368 bp, Artificial Sequence
  PSTVd variant
agatctcggaactaaactcgtggttcctgtggttcacacctgacctcctgacaagaaaagaaaaaagaaggcggctcggaggagcgcttcagggatccccggggaaacctggagcgaactggcaaaaaaggacggtggggagtgcccagcggccgacaggagtaattcccgccaaacagggttttcacctttcctttcttcgggtgtccttcctcgcgcccgcaggaccacccctggacccctttgcgctgtcgcttcggctactacccggtggaaacaactgaagctcccgagaaccgctttttctctatcttacttgctcgggcgagggtgtttagcccttggaaccgcagttggttcctagatct

SEQ ID NO: 11 - DNA, 358 bp, Artificial Sequence
  PSTVd variant
agatctcggaactaaactcgtggttcctgtggttcacacctgacctcctgacaagaaaagaaaaaagaaggcggctcggaggagcgcttcagggatccccggggaaacctggagcgaactggcaaaaaggacggtggggagtgcccagcggccgacaggagtaattcccgccgaaacagggttttcaccctttctttcttcgggtgtccttcctcgcgcccggaggaccacccctcgccccctttgcgctgtcgcttcggctactacccggtggaaacaactgaagctcccgagaaccgctttttctctatcttacgagggtgtttagcccttggaaccgcagttggttcctagatct

SEQ ID NO: 12 - DNA, 19 bp, Human immunodeficiency virus
atggagccagtagatccta

SEQ ID NO: 13 - DNA, 19 bp, Human immunodeficiency virus
ctagagccctggaagcatc

SEQ ID NO: 14 - DNA, 19 bp, Human immunodeficiency virus
tggcaggaagaagcggaga

SEQ ID NO: 15 - DNA, 29 bp, Artificial Sequence
  primer directed to human U6
tatgcacaattggtgctcgcttcggcagc

SEQ ID NO: 16 - DNA, 30 bp, Artificial Sequence
  primer directed to human U6
tgcacccaattgtatggaacgcttcacgaa

SEQ ID NO: 17 - DNA, 29 bp, Artificial Sequence
  primer directed to Influenza A
tcatcagaattcgcaggtaagtatcaagg

SEQ ID NO: 18 - DNA, 30 bp, Artificial Sequence
  primer directed to Influenza A
tggacaagatatcgacacctgtggagagaa

SEQ ID NO: 19 - DNA, 38 bp, Artificial Sequence
  primer directed to Influenza A
gatgcaggtaccgcggccgcgaactgagaagcaggtac

SEQ ID NO: 20 - DNA, 36 bp, Artificial Sequence
  primer directed to Influenza A
gatctacaattgcagctgtccttcattactcatgtc

SEQ ID NO: 21 - DNA, 33 bp, Artificial Sequence
  primer directed toward Influenza A
tcaatggcggccgccggaactaaactcgtggtt

SEQ ID NO: 22 - DNA, 31 bp, Artificial Sequence
  primer directed to Influenza A
caataggctagcaggaaccaactgcggttcc

SEQ ID NO: 23 - DNA, 31 bp, Artificial Sequence
  primer directed to human U6
tgaactgcggccgcgtgctcgcttcggcagc

SEQ ID NO: 24 - DNA, 30 bp, Artificial Sequence
  primer directed to human U6
acctgagctagctatggaacgcttcacgaa

SEQ ID NO: 25 - DNA, 178 bp, Artificial Sequence
  CUG repearting sequence of nucleic acid encoding humanized green fluorescent protein
atgcagctgctgcgctgctgctgctgctgctgctgctgctgctgctgctgctgctgctgctgctgctgctgctgctgctgctgctgcagctgctgctgctgctgctgctgctgctgctgctgctgctgctgctgctgctgctgctgctgctgctgctgctgctgtgctgctgctgcat

SEQ ID NO: 26 - RNA, 54 nt, Artificial Sequence
  “hairpin region” of nucleic acid directed to humanized green flurorescent protein
cugcugcugcugcugcugcugcugcugcugcugcugcugcugcugcugcugcug

SEQ ID NO: 27 - RNA, 107 nt, Homo sapiens
gugcucgcuucggcagcacauauacuaaaauuggaacgauacagagaagauuagcauggccccugcgcaaggaugacacgcaaauucgugaagcguuccauauuuuu